SEQUENCE LISTING (68 sequences)

SEQ ID NO: 1 - DNA, 372 bp, synthetic construct
  source 1..372: mol_type = other DNA
caggtgcagctggtggagtctgggggaggcgtggtccagcctgggaggtccctgagactctcctgtgcagcctctggattcaccttcagtagttatggcatgcactgggtccgccaggctccaggcaaggggctggagtgggtggcatttatgtcatatgatggaagtaataaattctattcagactccgtgaagggccgattcaccatctccagagacaattccaggaaaatgctgtttctgcaaatgaacaacctgagagctgaggacacggctgtgtattactgtgcaagagatcagtattacgattttttgactgatcacggggtctttgactactggggccagggaaccctggtcactgtctcctca

SEQ ID NO: 2 - protein, 124 aa, synthetic construct
QVQLVESGGGVVQPGRSLRLSCAASGFTFSSYGMHWVRQAPGKGLEWVAFMSYDGSNKFYSDSVKGRFTISRDNSRKMLFLQMNNLRAEDTAVYYCARDQYYDFLTDHGVFDYWGQGTLVTVSS

SEQ ID NO: 3 - DNA, 24 bp, synthetic construct
  source 1..24: mol_type = other DNA
ggattcaccttcagtagttatggc

SEQ ID NO: 4 - protein, 8 aa, synthetic construct
GFTFSSYG

SEQ ID NO: 5 - DNA, 24 bp, synthetic construct
  source 1..24: mol_type = other DNA
atgtcatatgatggaagtaataaa

SEQ ID NO: 6 - protein, 8 aa, synthetic construct
MSYDGSNK

SEQ ID NO: 7 - DNA, 51 bp, synthetic construct
  source 1..51: mol_type = other DNA
gcaagagatcagtattacgattttttgactgatcacggggtctttgactac

SEQ ID NO: 8 - protein, 17 aa, synthetic construct
ARDQYYDFLTDHGVFDY

SEQ ID NO: 9 - DNA, 369 bp, synthetic construct
  source 1..369: mol_type = other DNA
caggttcagctggtgcagtctggaactgaggtgaagaagcctggggcctcagtgaaggtctcctgcaaggcttctgattacacctttaccgactatggtatcacctgggtgcgacaggcccctggacaagggcttgagtggatgggatggatcagcccttacaatggtaacacagactatgcacagaagttccagggcagagtcaccatgaccgcagacacatccaggaccacagcctacatggagctgaggagcctgagatctgacgacacggccgtgtattactgtgcgagaaagggtcaactcgctctctactactattacggtttggacgtctggggccaagggaccacggtcaccgtctcctca

SEQ ID NO: 10 - protein, 123 aa, synthetic construct
QVQLVQSGTEVKKPGASVKVSCKASDYTFTDYGITWVRQAPGQGLEWMGWISPYNGNTDYAQKFQGRVTMTADTSRTTAYMELRSLRSDDTAVYYCARKGQLALYYYYGLDVWGQGTTVTVSS

SEQ ID NO: 11 - DNA, 24 bp, synthetic construct
  source 1..24: mol_type = other DNA
gattacacctttaccgactatggt

SEQ ID NO: 12 - protein, 8 aa, synthetic construct
DYTFTDYG

SEQ ID NO: 13 - DNA, 24 bp, synthetic construct
  source 1..24: mol_type = other DNA
atcagcccttacaatggtaacaca

SEQ ID NO: 14 - protein, 8 aa, synthetic construct
ISPYNGNT

SEQ ID NO: 15 - DNA, 48 bp, synthetic construct
  source 1..48: mol_type = other DNA
gcgagaaagggtcaactcgctctctactactattacggtttggacgtc

SEQ ID NO: 16 - protein, 16 aa, synthetic construct
ARKGQLALYYYYGLDV

SEQ ID NO: 17 - DNA, 324 bp, synthetic construct
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 18 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 19 - DNA, 21 bp, synthetic construct
  source 1..21: mol_type = other DNA
cagtcagtctctagctcttat

SEQ ID NO: 20 - protein, 7 aa, synthetic construct
QSVSSSY

SEQ ID NO: 21-22 - skipped (2 entries)

SEQ ID NO: 23 - DNA, 27 bp, synthetic construct
  source 1..27: mol_type = other DNA
caacagtacggaagcagcccgtggacg

SEQ ID NO: 24 - protein, 9 aa, synthetic construct
QQYGSSPWT

SEQ ID NO: 25 - DNA, 1353 bp, synthetic construct
  source 1..1353: mol_type = other DNA
caggtgcagctggtggagtctgggggaggcgtggtccagcctgggaggtccctgagactctcctgtgcagcctctggattcaccttcagtagttatggcatgcactgggtccgccaggctccaggcaaggggctggagtgggtggcatttatgtcatatgatggaagtaataaattctattcagactccgtgaagggccgattcaccatctccagagacaattccaggaaaatgctgtttctgcaaatgaacaacctgagagctgaggacacggctgtgtattactgtgcaagagatcagtattacgattttttgactgatcacggggtctttgactactggggccagggaaccctggtcactgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 26 - protein, 450 aa, synthetic construct
QVQLVESGGGVVQPGRSLRLSCAASGFTFSSYGMHWVRQAPGKGLEWVAFMSYDGSNKFYSDSVKGRFTISRDNSRKMLFLQMNNLRAEDTAVYYCARDQYYDFLTDHGVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 2064 bp, synthetic construct
  source 1..2064: mol_type = other DNA
caggtgcaactcgtgcagtctgggactgaggttaaaaaacctggtgcctccgtcaaagtgtcttgtaaagcatcagattacacttttaccgattatggaatcacatgggttagacaggctcctggacagggattggaatggatgggatggatttctccttacaatggtaacaccgattacgcacaaaaatttcaaggccgcgtgactatgacagcagacacttcccgaactaccgcttacatggaactcaggtccttgcgttctgacgataccgcagtttattattgtgccaggaagggacagttggcactctactattactatggtctggatgtgtgggggcagggcactaccgtcaccgtgagctcagcatctacaaaagggccatccgttttccctctggcaccctgttcaagatctacctctgaatctacagccgctctcggatgcctcgtgaaagactatttcccagaacccgtcaccgtttcatggaacagcggcgccctcacatccggcgtgcatacctttcctgcagtgctccaatcttccggtctgtattctctgtctagtgtcgtgactgttccatcatcttccctcggaactaaaacctatacctgtaacgttgaccataagccctctaacactaaggtggataaaagagtcgaatccaagtacggccctccaggcggcggtggatctggaggcggtggaagccaggttcagctggtgcagtctggaactgaggtgaagaagcctggggcctcagtgaaggtctcctgcaaggcttctgattacacctttaccgactatggtatcacctgggtgcgacaggcccctggacaagggcttgagtggatgggatggatcagcccttacaatggtaacacagactatgcacagaagttccagggcagagtcaccatgaccgcagacacatccaggaccacagcctacatggagctgaggagcctgagatctgacgacacggccgtgtattactgtgcgagaaagggtcaactcgctctctactactattacggtttggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 28 - protein, 687 aa, synthetic construct
QVQLVQSGTEVKKPGASVKVSCKASDYTFTDYGITWVRQAPGQGLEWMGWISPYNGNTDYAQKFQGRVTMTADTSRTTAYMELRSLRSDDTAVYYCARKGQLALYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGGGGSGGGGSQVQLVQSGTEVKKPGASVKVSCKASDYTFTDYGITWVRQAPGQGLEWMGWISPYNGNTDYAQKFQGRVTMTADTSRTTAYMELRSLRSDDTAVYYCARKGQLALYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 29 - DNA, 648 bp, synthetic construct
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 30 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 351 bp, synthetic construct
  source 1..351: mol_type = other DNA
gagtttcaggtggtggagtctgggggaggcctggtcaagcctggggggtccctcagactctcctgtgtggtctccggattcaccttcagtaactataatatgaactgggtccgccaggctccagcgaagggactggagtgggtctcatccattagtactggtagtagtgacatttactacgcagactcagtgaagggccgattcaccatctccagagacaacgccaagaactcactgtatctgcaaatgaacagcctgagagtcgaggacacggctgtttattactgtgcgagagatataattggaactacgagggactggggccagggaaccctggtcaccgtctcctca

SEQ ID NO: 32 - protein, 117 aa, synthetic construct
EFQVVESGGGLVKPGGSLRLSCVVSGFTFSNYNMNWVRQAPAKGLEWVSSISTGSSDIYYADSVKGRFTISRDNAKNSLYLQMNSLRVEDTAVYYCARDIIGTTRDWGQGTLVTVSS

SEQ ID NO: 33 - DNA, 24 bp, synthetic construct
  source 1..24: mol_type = other DNA
ggattcaccttcagtaactataat

SEQ ID NO: 34 - protein, 8 aa, synthetic construct
GFTFSNYN

SEQ ID NO: 35 - DNA, 24 bp, synthetic construct
  source 1..24: mol_type = other DNA
attagtactggtagtagtgacatt

SEQ ID NO: 36 - protein, 8 aa, synthetic construct
ISTGSSDI

SEQ ID NO: 37 - DNA, 30 bp, synthetic construct
  source 1..30: mol_type = other DNA
gcgagagatataattggaactacgagggac

SEQ ID NO: 38 - protein, 10 aa, synthetic construct
ARDIIGTTRD

SEQ ID NO: 39 - DNA, 381 bp, synthetic construct
  source 1..381: mol_type = other DNA
gaagtgcagctggtggagtctgggggaggcctggtacagcctggcaggtccctgagactctcctgtggagcctctggattcacctttgatgattatgtcatgcactgggtccggcaaattccagggaagggcctggagtgggtcgcaggtattagttggaatagtggtagtataggctatgcggactctgtgaagggccgattcaccatctccagagacaacgccaagaactccctgtatctgcaaatgaacagtctgagagctgaggacacggccttgtatttctgtgcaaaagatcatattgacccgtatagcagcagcctctactactactatggtatggacgtctggggccaagggaccacggtcaccgtctcctca

SEQ ID NO: 40 - protein, 127 aa, synthetic construct
EVQLVESGGGLVQPGRSLRLSCGASGFTFDDYVMHWVRQIPGKGLEWVAGISWNSGSIGYADSVKGRFTISRDNAKNSLYLQMNSLRAEDTALYFCAKDHIDPYSSSLYYYYGMDVWGQGTTVTVSS

SEQ ID NO: 41 - DNA, 24 bp, synthetic construct
  source 1..24: mol_type = other DNA
ggattcacctttgatgattatgtc

SEQ ID NO: 42 - protein, 8 aa, synthetic construct
GFTFDDYV

SEQ ID NO: 43 - DNA, 24 bp, synthetic construct
  source 1..24: mol_type = other DNA
attagttggaatagtggtagtata

SEQ ID NO: 44 - protein, 8 aa, synthetic construct
ISWNSGSI

SEQ ID NO: 45 - DNA, 60 bp, synthetic construct
  source 1..60: mol_type = other DNA
gcaaaagatcatattgacccgtatagcagcagcctctactactactatggtatggacgtc

SEQ ID NO: 46 - protein, 20 aa, synthetic construct
AKDHIDPYSSSLYYYYGMDV

SEQ ID NO: 47 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 48 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 49 - DNA, 18 bp, synthetic construct
  source 1..18: mol_type = other DNA
cagagcattagcagctat

SEQ ID NO: 50 - protein, 6 aa, synthetic construct
QSISSY

SEQ ID NO: 51-52 - skipped (2 entries)

SEQ ID NO: 53 - DNA, 30 bp, synthetic construct
  source 1..30: mol_type = other DNA
caacagagttacagtacccctccgatcacc

SEQ ID NO: 54 - protein, 10 aa, synthetic construct
QQSYSTPPIT

SEQ ID NO: 55 - DNA, 1332 bp, synthetic construct
  source 1..1332: mol_type = other DNA
gagtttcaggtggtggagtctgggggaggcctggtcaagcctggggggtccctcagactctcctgtgtggtctccggattcaccttcagtaactataatatgaactgggtccgccaggctccagcgaagggactggagtgggtctcatccattagtactggtagtagtgacatttactacgcagactcagtgaagggccgattcaccatctccagagacaacgccaagaactcactgtatctgcaaatgaacagcctgagagtcgaggacacggctgtttattactgtgcgagagatataattggaactacgagggactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 56 - protein, 443 aa, synthetic construct
EFQVVESGGGLVKPGGSLRLSCVVSGFTFSNYNMNWVRQAPAKGLEWVSSISTGSSDIYYADSVKGRFTISRDNAKNSLYLQMNSLRVEDTAVYYCARDIIGTTRDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - DNA, 2088 bp, synthetic construct
  source 1..2088: mol_type = other DNA
gaagtgcagctggtggagtctgggggaggcctggtacagcctggcaggtccctgagactctcctgtggagcctctggattcacctttgatgattatgtcatgcactgggtccggcaaattccagggaagggcctggagtgggtcgcaggtattagttggaatagtggtagtataggctatgcggactctgtgaagggccgattcaccatctccagagacaacgccaagaactccctgtatctgcaaatgaacagtctgagagctgaggacacggccttgtatttctgtgcaaaagatcatattgacccgtatagcagcagcctctactactactatggtatggacgtctggggccaagggaccacggtcaccgtctcctcagcatctacaaaagggccatccgttttccctctggcaccctgttcaagatctacctctgaatctacagccgctctcggatgcctcgtgaaagactatttcccagaacccgtcaccgtttcatggaacagcggcgccctcacatccggcgtgcatacctttcctgcagtgctccaatcttccggtctgtattctctgtctagtgtcgtgactgttccatcatcttccctcggaactaaaacctatacctgtaacgttgaccataagccctctaacactaaggtggataaaagagtcgaatccaagtacggccctccaggcggcggtggatctggaggcggtggaagcgaagttcaactcgttgagtcaggaggaggactcgtacaaccaggaagatctttgcgtctgtcatgcggtgcttctggatttacatttgatgattacgtaatgcattgggtcagacaaatcccaggtaaaggactggaatgggttgccggaattagttggaacagtggctctataggatatgcagattccgtcaaaggtcgattcactatatctcgtgataacgctaaaaactctctctatctccaaatgaatagccttcgcgctgaagatactgccctttacttttgcgctaaagaccatatagacccatatagttcctctctgtactactactatggcatggacgtctggggccaaggtacaactgttactgtgtcaagtgcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 58 - protein, 695 aa, synthetic construct
EVQLVESGGGLVQPGRSLRLSCGASGFTFDDYVMHWVRQIPGKGLEWVAGISWNSGSIGYADSVKGRFTISRDNAKNSLYLQMNSLRAEDTALYFCAKDHIDPYSSSLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGGGGSGGGGSEVQLVESGGGLVQPGRSLRLSCGASGFTFDDYVMHWVRQIPGKGLEWVAGISWNSGSIGYADSVKGRFTISRDNAKNSLYLQMNSLRAEDTALYFCAKDHIDPYSSSLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 59 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 60 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 369 bp, synthetic construct
  source 1..369: mol_type = other DNA
caggtgcaactcgtgcagtctgggactgaggttaaaaaacctggtgcctccgtcaaagtgtcttgtaaagcatcagattacacttttaccgattatggaatcacatgggttagacaggctcctggacagggattggaatggatgggatggatttctccttacaatggtaacaccgattacgcacaaaaatttcaaggccgcgtgactatgacagcagacacttcccgaactaccgcttacatggaactcaggtccttgcgttctgacgataccgcagtttattattgtgccaggaagggacagttggcactctactattactatggtctggatgtgtgggggcagggcactaccgtcaccgtgagctca

SEQ ID NO: 62 - DNA, 24 bp, synthetic construct
  source 1..24: mol_type = other DNA
gattacacttttaccgattatgga

SEQ ID NO: 63 - DNA, 24 bp, synthetic construct
  source 1..24: mol_type = other DNA
atttctccttacaatggtaacacc

SEQ ID NO: 64 - DNA, 48 bp, synthetic construct
  source 1..48: mol_type = other DNA
gccaggaagggacagttggcactctactattactatggtctggatgtg

SEQ ID NO: 65 - DNA, 381 bp, synthetic construct
  source 1..381: mol_type = other DNA
gaagttcaactcgttgagtcaggaggaggactcgtacaaccaggaagatctttgcgtctgtcatgcggtgcttctggatttacatttgatgattacgtaatgcattgggtcagacaaatcccaggtaaaggactggaatgggttgccggaattagttggaacagtggctctataggatatgcagattccgtcaaaggtcgattcactatatctcgtgataacgctaaaaactctctctatctccaaatgaatagccttcgcgctgaagatactgccctttacttttgcgctaaagaccatatagacccatatagttcctctctgtactactactatggcatggacgtctggggccaaggtacaactgttactgtgtcaagt

SEQ ID NO: 66 - DNA, 24 bp, synthetic construct
  source 1..24: mol_type = other DNA
ggatttacatttgatgattacgta

SEQ ID NO: 67 - DNA, 24 bp, synthetic construct
  source 1..24: mol_type = other DNA
attagttggaacagtggctctata

SEQ ID NO: 68 - DNA, 60 bp, synthetic construct
  source 1..60: mol_type = other DNA
gctaaagaccatatagacccatatagttcctctctgtactactactatggcatggacgtc

(19) United States
(12) Patent Application Publication
(10) Pub. No.: US 2026/0116993 A1
(43) Pub. Date: Apr. 30, 2026

(54) MULTISPECIFIC ANTIGEN-BINDING MOLECULES THAT BIND PSMA AND 4-1BB AND METHODS OF USE THEREOF

(71) Applicant: Regeneron Pharmaceuticals, Inc., Tarrytown, NY (US)

(72) Inventors: Janelle Waite, Bronx, NY (US); Lauric Haber, Rye Brook, NY (US); Erica Ullman, Yorktown Heights, NY (US)

(21) Appl. No.: 19/372,639

(22) Filed: Oct. 29, 2025

Related U.S. Application Data
(60) Provisional application No. 63/713,813, filed on Oct. 30, 2024.

(51) Int. Cl. C07K 16/30; A61K 38/00; A61P 35/00; C07K 16/28; C12N 15/11; C12N 15/70; C12N 15/85

(52) U.S. Cl. CPC C07K 16/3069; A61K 38/00; A61P 35/00; C07K 16/2809; C07K 16/2818; C07K 16/2878; C12N 15/11; C12N 15/70; C12N 15/85; C07K 2317/31; C07K 2317/32; C07K 2317/33; C07K 2317/565

(57) ABSTRACT

The present disclosure provides multispecific antigen-binding molecules that bind to prostate specific membrane antigen (PSMA) and 4-1BB (anti-PSMA x anti-4-1BB). In certain embodiments, the present disclosure provides a multispecific anti-PSMA x anti-4-1BB antigen-binding molecule or antigen-binding fragment thereof comprising a first antigen-binding domain that specifically binds PSMA, a second antigen-binding domain that specifically binds 4-1BB, and a third antigen-binding domain that specifically binds 4-1BB. In certain embodiments, the molecules are multispecific antibodies or antigen-binding fragments thereof. In certain embodiments, the multispecific antibodies disclosed herein bind PSMA expressed on tumor cells with the first antigen-binding domain and 4-1BB on immune cells (e.g., T cells) with the second and third antigen-binding domains. In certain embodiments, the antibodies of the present disclosure are useful in treatment of a cancer. In certain embodiments, the antibodies are useful for treating a PSMA-associated disease or disorder (e.g., prostate cancer).

### FIELD

The present disclosure relates to multispecific antigen-binding molecules that bind to PSMA and 4-1BB and methods of use thereof, e.g., for treating or preventing cancer.

### SEQUENCE LISTING

The instant application contains a Sequence Listing which has been submitted electronically in XML format and is hereby incorporated by reference in its entirety. Said XML copy, created on Oct. 24, 2025, is named SeqList11618 and is 72, 107 bytes in size. The sequence listing is part of the specification and is incorporated in its entirety by reference herein.

### BACKGROUND

The ability of T cells to recognize and kill their cellular targets, such as virally-infected cells or tumor cells, depends on a coordinated set of interactions. Two signals, “signal 1” & “signal 2”, are required for proper T cell activation. Signal 1 involves recognition and binding of the target cell by the T cell Receptor (TCR) complex (which includes the associated CD3 gamma (γ), delta (δ), epsilon (ε), and zeta (λ) chains). The TCR recognizes a viral or tumor peptide presented on the groove of an MHC protein expressed on the surface of a target cell. Because such binding is generally of low-affinity, successful triggering of signal 1 requires clustering of many TCR complexes along the interface between the T cell and its target cell. This interface has been referred to as the “immune synapse.” Signal 2 is provided by engaging co-stimulatory receptors on T cells. One such costimulatory receptor is 4-1BB, which is an inducible type I membrane protein and member of the tumor necrosis factor receptor (TNFR) superfamily. Expression of 4-1BB receptor is induced on the surface of T cells after antigen- or mitogen-induced activation. When a T cell recognizes its target cell via its TCR complex, and then also engages signal 2 via 4-1BB binding to its cognate ligand(s) on the target cell, T cell activation is enhanced.

4-1BB has been garnering attention as a promising therapeutic target in the setting of cancer, amongst other diseases, due to its broad expression profile and ability to stimulate various signaling pathways involved in the generation of a potent immune response. As a prominent mediator of immune responses expressed on various cell types, 4-1BB signaling not only exerts protective effects, but is also capable of driving pathologies such as the adverse effects observed following administration of therapeutic 4-1BB antibodies. The persistent stimulation of 4-1BB signaling and, consequently, continuous activation of T cells have been shown to result in granuloma formation in tumor-draining lymph nodes due to the excessive recruitment of macrophages (Kim et al. Cell Mol. Immunol. (2021) 18 (8): 1956-68). Furthermore, liver-associated toxicity has been reported to be a common problem associated with therapeutic 4-1BB antibody treatment. Urelumab (BMS-663513), a fully human IgG4 monoclonal antibody, was the first anti-4-1BB therapeutic to enter clinical trials. Clinical development halted when liver toxicity associated with the antibody was revealed. It has been showed that 4-1BB antibody treatment results in CD8⁺ T cell infiltration into the liver causing inflammation and increased transaminase expression (Dubrot et al. Cancer Immunol. Immunother. (2010) 59 (8): 1223-33). Such infiltration, however, was not associated with clinical benefit in the setting of tumors in or around the liver tissue. Localized or targeted use of anti-4-1BB mAb can be used for promotion of antitumor immunity with less risk.

Prostate-specific membrane antigen (PSMA, also known as FOLH1) is an integral, non-shed membrane glycoprotein highly expressed on malignant prostate tissue and is a cell-surface marker for prostate cancer but shows limited expression on normal tissue. PSMA is also expressed within the neovasculature of other tumors such as bladder, lung, renal, gastric, and colorectal carcinomas.

Prostate cancer is the leading cause of new cancer diagnoses and the second most common cause of cancer-related death in men in the United States. There were 1.3 million new cases of prostate cancer and 358,989 deaths estimated worldwide in 2018. Methods for treating prostate cancer by targeting PSMA have been investigated. For example, Yttrium-90 capromab is a radiotherapeutic comprising a monoclonal antibody to an intracellular epitope of PSMA. In another example, J591, a monoclonal antibody to an extracellular epitope of PSMA, is part of the radiotherapeutic Lutetium-177 J591 and in MLN2704, in which maytansinoid 1 (DM1, an antimicrotubule agent) is conjugated to J591. These therapies have been associated with toxicity.

Monoclonal antibodies (mAbs) aimed at enhancing T-cell activation are under clinical development as anti-tumor therapeutics. The majority of current treatments, however, have a difficult time overcoming the inhibitory nature of the tumor microenvironment, thus failing to generate efficient tumor-specific T-cell activation and subsequent tumor cell killing. Several blocking mAbs directed against checkpoint inhibitors such as CTLA-4 (cytotoxic T lymphocyte-associated protein) and programmed cell death 1 (PD-1)/programmed cell death ligand 1 (PSMA) have been clinically approved for melanoma, renal cell carcinoma, non-small lung cancer and advanced metastatic cutaneous squamous cell carcinoma. Blocking PD-1 releases the break on T-cell activation, but its efficacy as a single agent often it is not sufficient to achieve tumor clearance and durable anti-tumor responses. Therefore, there is a need for additional treatments for cancer, such as improved immunotherapeutic agents.

### SUMMARY

In one aspect, the disclosed technology relates to a multispecific antigen-binding molecule comprising: (a) a first antigen-binding domain (D1) comprising three complementarity determining regions (CDRs) of a heavy chain variable region (D1-HCVR) and three CDRs of a light chain variable region (D1-LCVR), wherein the first antigen-binding domain binds specifically to PSMA; and (b) a second antigen-binding domain (D2) comprising three CDRs of a HCVR (D2-HCVR) and three CDRs of a LCVR (D2-LCVR), wherein the second antigen-binding domain binds specifically to 4-1BB; and (c) a third antigen-binding domain (D3) comprising three CDRs of a HCVR (D3-HCVR) and three CDRs of a LCVR (D3-LCVR), wherein the third antigen-binding domain binds specifically to 4-1BB.

In some embodiments, D2 and D3 bind to the same epitope on 4-1BB. In some embodiments, D2 and D3 bind to different epitopes on 4-1BB. In some embodiments, D1 comprises three CDRs of a HCVR comprising an amino acid sequence selected from the group consisting of SEQ ID NOs: 2 and 32. In some embodiments, wherein D1 comprises three CDRs of a LCVR comprising an amino acid sequence selected from the group consisting of SEQ ID NOs: 18 and 48. In some embodiments, D1 comprises three heavy chain complementarity determining regions (HCDR2, HCDR2, and HCDR3) comprising an amino acid sequence selected from the group consisting of SEQ ID NO: 4, SEQ ID NO: 6, and SEQ ID NO: 8; and SEQ ID NO: 34, SEQ ID NO: 36, and SEQ ID NO: 38, respectively. In some embodiments, D1 comprises three light chain complementarity determining regions (LCDR2, LCDR2, and LCDR3) comprising an amino acid sequence selected from the group consisting of SEQ ID NO: 20, GAS, and SEQ ID NO: 24; and SEQ ID NO: 50, AAS, and SEQ ID NO: 54, respectively. In some embodiments, D1 comprises a HCVR comprising the amino acid sequence selected from the group consisting of SEQ ID NOs: 2 and 32. In some embodiments, D1 comprises a LCVR comprising the amino acid sequence selected from the group consisting of SEQ ID NOs: 18 and 48. In some embodiments, wherein D1 comprises a HCVR comprising the amino acid sequence selected from the group consisting of SEQ ID NOs: 2 and 32; and a LCVR comprising the amino acid sequence selected from the group consisting of SEQ ID NOs: 18 and 48.

In some embodiments, D2 comprises three CDRs of a HCVR (D2-HCVR) comprising an amino acid sequence selected from the group consisting of SEQ ID NOs: 10 and 40. In some embodiments, wherein D2 comprises three CDRs of a LCVR (D2-LCVR) comprising an amino acid sequence selected from the group consisting of SEQ ID NOs: 18 and 48. In some embodiments, D2 comprises three heavy chain complementarity determining regions (D2-HCDR1, D2-HCDR2, and D2-HCDR3) comprising an amino acid sequence selected from the group consisting of SEQ ID NO: 12, SEQ ID NO: 14, and, SEQ ID NO: 16; and SEQ ID NO: 42, SEQ ID NO: 44, and SEQ ID NO: 46, respectively. In some embodiments, wherein D2 comprises three light chain complementarity determining regions (D2-LCDR1, D2-LCDR2, and R1-LCDR3) comprising an amino acid sequence selected from the group consisting of SEQ ID NO: 20, GAS, and SEQ ID NO: 24; and SEQ ID NO: 50, AAS, and SEQ ID NO: 54, respectively. In some embodiments, wherein D2 comprises a HCVR (D2-HCVR) comprising the amino acid sequence selected from the group consisting of SEQ ID NOs: 10 and 40. In some embodiments, D2 comprises a LCVR (D2-LCVR) comprising the amino acid sequence selected from the group consisting of SEQ ID NOs: 18 and 48. In some embodiments, D2 comprises a D2-HCVR comprising the amino acid sequence selected from the group consisting of SEQ ID NOs: 10 and 40; and a D2-LCVR comprising the amino acid sequence selected from the group consisting of SEQ ID NOs: 18 and 48.

In some embodiments, D3 comprises three CDRs of a HCVR (D3-HCVR) comprising the amino acid sequence selected from the group consisting of SEQ ID NOs: 10 and 40. In some embodiments, D3 comprises three CDRs of a LCVR (D3-LCVR) comprising the amino acid sequence selected from the group consisting of SEQ ID NOs: 18 and 48. In some embodiments, D3 comprises three heavy chain complementarity determining regions (D3-HCDR1, D3-HCDR2, and D3-HCDR3) comprising an amino acid sequence selected from the group consisting of SEQ ID NO: 12, SEQ ID NO: 14, and SEQ ID NO: 16; and SEQ ID NO: 42, SEQ ID NO: 44, and SEQ ID NO: 46, respectively. In some embodiments, D3 comprises three light chain complementarity determining regions (D3-LCDR1, D3-LCDR2, and D3-LCDR3) comprising an amino acid sequence selected from the group consisting of SEQ ID NO: 20, GAS, and SEQ ID NO: 24; and SEQ ID NO: 50, AAS, and SEQ ID NO: 54, respectively. In some embodiments, D3 comprises a HCVR (D3-HCVR) comprising the amino acid sequence selected from the group consisting of SEQ ID NOs: 10 and 40. In some embodiments, D3 comprises a LCVR (D3-LCVR) comprising the amino acid sequence selected from the group consisting of SEQ ID NOs: 18 and 48. In some embodiments, D3 comprises a D3-HCVR comprising the amino acid sequence selected from the group consisting of SEQ ID NOs: 10 and 40; and a D3-LCVR comprising the amino acid sequence selected from the group consisting of SEQ ID NOs: 18 and 48.

In some embodiments, (a) D1 comprises three CDRs of D1-HCVR comprising the amino acid sequence of SEQ ID NO: 2, and three CDRs of D1-LCVR comprising the amino acid sequence of SEQ ID NO: 18; and (b) D2 comprises three CDRs of D2-HCVR comprising an amino acid sequence selected from the group consisting of SEQ ID NOs: 10 and 40; and D2-LCVR comprising an amino acid sequence selected from the group consisting of SEQ ID NOS: 18 and 48; and (c) D3 comprises three CDRs of D3-HCVR comprising an amino acid sequence selected from the group consisting of SEQ ID NOs: 10 and 40 and D3-LCVR comprising an amino acid sequence selected from the group consisting of SEQ ID NOs: 18 and 48.

In some embodiments, (a) the first antigen-binding domain (D1) comprises three CDRs of D1-HCVR comprising the amino acid sequence of SEQ ID NO: 32, and three CDRs of D1-LCVR comprising the amino acid sequence of SEQ ID NO: 48; and (b) a second antigen-binding domain (D2) comprising three CDRs of D2-HCVR comprising an amino acid sequence selected from the group consisting of SEQ ID NOs: 10 and 40; and D2-LCVR comprising an amino acid sequence selected from the group consisting of SEQ ID NOs: 18 and 48; and (c) a third antigen-binding domain (D3) comprising three CDRs of D3-HCVR comprising an amino acid sequence selected from the group consisting of SEQ ID NOs: 10 and 40 and D3-LCVR comprising an amino acid sequence selected from the group consisting of SEQ ID NOs: 18 and 48. In some embodiments, (a) the first antigen-binding domain (D1) comprises three CDRs of D1-HCVR comprising the amino acid sequence of SEQ ID NO: 2, and three CDRs of D1-LCVR comprising the amino acid sequence of SEQ ID NO: 18; and (b) the second antigen-binding domain (D2) comprises three CDRs of D2-HCVR comprising the amino acid sequence of SEQ ID NO: 10; and three CDRs of D2-LCVR comprising the amino acid sequence of SEQ ID NO: 18; and (c) a third antigen-binding domain (D3) comprising three CDRs of D3-HCVR comprising the amino acid sequence of SEQ ID NO: 10; and three CDRs of D3-LCVR comprising the amino acid sequence of SEQ ID No: 18.

In some embodiments, (a) the first antigen-binding domain (D1) comprises D1-HCDR1, D1-HCDR2, D1-HCDR3, D1-LCDR1, D1-LCDR2, and D1-LCDR3 comprising the respective amino acid sequences of SEQ ID NO: 4, SEQ ID NO: 6, SEQ ID NO: 8, SEQ ID NO: 20, GAS, and SEQ ID NO: 24; and (b) the second antigen-binding domain (D2) comprises D2-HCDR1, D2-HCDR2, D2-HCDR3, D2-LCDR1, D2-LCDR2, and D2-LCDR3 comprising the respective amino acid sequences of SEQ ID NO: 12, SEQ ID NO: 14, SEQ ID NO: 16, SEQ ID NO: 20, GAS, and SEQ ID NO: 24; and (c) a third antigen-binding domain (D3) comprising D3-HCDR1, D3-HCDR2, D3-HCDR3, D3-LCDR1, D3-LCDR2, and D3-LCDR3 comprising the respective amino acid sequences of SEQ ID NO: 12, SEQ ID NO: 14, SEQ ID NO: 16, SEQ ID NO: 20, GAS, and SEQ ID NO: 24.

In some embodiments, (a) the first antigen-binding domain (D1) comprises three CDRs of D1-HCVR comprising the amino acid sequence of SEQ ID NO: 32, and three CDRs of D1-LCVR comprising the amino acid sequence of SEQ ID NO: 48; and (b) the second antigen-binding domain (D2) comprises three CDRs of D2-HCVR comprising the amino acid sequence of SEQ ID NO: 40; and three CDRs of D2-LCVR comprising the amino acid sequence of SEQ ID NO: 48; and (c) a third antigen-binding domain (D3) comprising three CDRs of D3-HCVR comprising the amino acid sequence of SEQ ID NO: 40; and three CDRs of D3-LCVR comprising the amino acid sequence of SEQ ID No: 48.

In some embodiments, (a) the first antigen-binding domain (D1) comprises D1-HCDR1, D1-HCDR2, D1-HCDR3, D1-LCDR1, D1-LCDR2, and D1-LCDR3 comprising the respective amino acid sequences of SEQ ID NO: 34, SEQ ID NO: 36, SEQ ID NO: 38, SEQ ID NO: 50, AAS, and SEQ ID NO: 54; and (b) the second antigen-binding domain (D2) comprises D2-HCDR1, D2-HCDR2, D2-HCDR3, D2-LCDR1, D2-LCDR2, and D2-LCDR3 comprising the respective amino acid sequences of SEQ ID NO: 42, SEQ ID NO: 44, SEQ ID NO: 46, SEQ ID NO: 50, AAS, and SEQ ID NO: 54; and (c) a third antigen-binding domain (D3) comprising D3-HCDR1, D3-HCDR2, D3-HCDR3, D3-LCDR1, D3-LCDR2, and D3-LCDR3 comprising the respective amino acid sequences of SEQ ID NO: 42, SEQ ID NO: 44, SEQ ID NO: 46, SEQ ID NO: 50, AAS, and SEQ ID NO: 54.

In some embodiments, the molecule is a multispecific antibody comprising: (a) a first antigen-binding arm comprising a first antigen-binding domain (D1) comprising three CDRs (D1-HCDR1, D1-HCDR2, and D1-HCDR3) of a HCVR (D1-HCVR) and three CDRs (D1-LCDR1, D1-LCDR2, and D1-LCDR3) of a LCVR (D1-LCVR), wherein the first antigen-binding arm binds specifically to PSMA; and (b) a second antigen-binding arm comprising (i) a second antigen-binding domain (D2) comprising three CDRs (D2-HCDR1, D2-HCDR2, and D2-HCDR3) of a HCVR (D2-HCVR) and three CDRs (D2-LCDR1, D2-LCDR2, and D2-LCDR3) of a LCVR (D2-LCVR); and (ii) a third antigen-binding domain (D3) comprising three CDRs (D3-HCDR1, D3-HCDR2, and D3-HCDR3) of a HCVR (D3-HCVR) and three CDRs (D3-LCDR1, D3-LCDR2, and D3-LCDR3) of a LCVR (D3-LCVR), wherein the second antigen-binding arm binds specifically to 4-1BB.

In some embodiments, the multispecific antibody comprises a heavy chain, wherein the heavy chain constant region is of IgG1 or IgG4 isotype. In some embodiments, the multispecific antibody comprises a first heavy chain comprising D1-HCVR, and a second heavy chain comprising D2-HCVR and D3-HCVR; wherein the second heavy chain comprises the mutations H435R and Y436F (EU numbering).

In some embodiments, the multispecific antibody comprises a first heavy chain comprising the D1-HCVR of the first antigen-binding arm paired with a light chain comprising D1-LCVR of the first antigen-binding arm, wherein the first heavy chain comprises the amino acid sequence of SEQ ID NO: 26 and the light chain comprises the amino acid sequence of SEQ ID NO: 30. In some embodiments, the multispecific antibody comprises a second heavy chain comprising D2-HCVR and D3-HCVR of the second antigen-binding arm paired with a second light chain comprising D2-LCVR and a third light chain comprising D3-LCVR, wherein the second heavy chain comprises the amino acid sequence of SEQ ID NO: 28; the second light chain comprises the amino acid sequence of SEQ ID NO: 30, and the third light chain comprises the amino acid sequence of SEQ ID NO: 30. In some embodiments, the multispecific antibody comprises a first heavy chain comprising D1-HCVR of the first antigen-binding arm paired with a first light chain comprising D1-LCVR of the first antigen-binding arm, wherein the first heavy chain comprises the amino acid sequence of SEQ ID NO: 56, and the first light chain comprises the amino acid sequence of SEQ ID NO: 60. In some embodiments, the multispecific antibody comprises a second heavy chain comprising D2-HCVR and D3-HCVR of the second antigen-binding arm paired with a second light chain comprising D2-LCVR and a third light chain comprising D3-LCVR, wherein the second heavy chain comprises the amino acid sequence of SEQ ID NO: 58; the second light chain comprises the amino acid sequence of SEQ ID NO: 60, and the third light chain comprises the amino acid sequence of SEQ ID NO: 60.

In some embodiments, the multispecific antibody comprises a first antigen-binding arm comprising a heavy chain comprising the sequence of SEQ ID NO: 26 and a first light chain comprising the sequence of SEQ ID NO 30; and the second antigen-binding arm comprises a heavy chain comprising the sequence of SEQ ID NO: 28, a second light chain comprising the sequence of SEQ ID NO: 30, and a third light chain comprising the sequence of SEQ ID NO: 30. In some embodiments, the multispecific antibody comprises a first antigen-binding arm that comprises a heavy chain comprising the sequence of SEQ ID NO: 56 and a first light chain comprising the sequence of SEQ ID NO 60; and the second antigen-binding arm comprises a heavy chain comprising the sequence of SEQ ID NO: 58, a second light chain comprising the sequence of SEQ ID NO: 60, and a third light chain comprising the sequence of SEQ ID NO: 60.

In some embodiments, the multispecific antibody comprises a first antigen-binding arm that binds specifically to PSMA and a second antigen-binding arm that binds specifically to 4-1BB, wherein the first antigen-binding arm comprises a first antigen-binding domain (D1) comprising three CDRs of a HCVR (D1-HCVR) comprising the amino acid sequence of SEQ ID NO: 2, and three CDRs of a LCVR (D1-LCVR) comprising the amino acid sequence of SEQ ID NO: 18; and the second antigen-binding arm comprises a second antigen-binding domain (D2) comprising (i) three CDRs of a HCVR (D2-HCVR) comprising the amino acid sequence of SEQ ID NO: 10, and three CDRs of a LCVR (D2-LCVR) comprising the amino acid sequence of SEQ ID NO: 18, and (ii) a third antigen-binding domain (D3) comprising (i) three CDRs of a HCVR (D3-HCVR) comprising the amino acid sequence of SEQ ID NO: 10, and three CDRs of a LCVR (D3-LCVR) comprising the amino acid sequence of SEQ ID NO: 18. In some embodiments, the multispecific antibody comprises a first antigen-binding arm that binds specifically to PSMA and a second antigen-binding arm that binds specifically to 4-1BB, wherein the first antigen-binding arm comprises a first antigen-binding domain (D1) comprising three CDRs of a HCVR (D1-HCVR) comprising the amino acid sequence of SEQ ID NO: 32, and three CDRs of a LCVR (D1-LCVR) comprising the amino acid sequence of SEQ ID NO: 48; and the second antigen-binding arm comprises a second antigen-binding domain (D2) comprising (i) three CDRs of a HCVR (D2-HCVR) comprising the amino acid sequence of SEQ ID NO: 40, and three CDRs of a LCVR (D2-LCVR) and comprising the amino acid sequence of SEQ ID NO: 48, and (ii) a third antigen-binding domain (D3) comprising (i) three CDRs of a HCVR (D3-HCVR) comprising the amino acid sequence of SEQ ID NO: 40, and three CDRs of a LCVR (D3-LCVR) comprising the amino acid sequence of SEQ ID NO: 48.

In some embodiments, the multispecific antibody comprises a first heavy chain comprising the HCVR of the first antigen-binding arm, wherein the first heavy chain is paired with a light chain comprising the LCVR of the first antigen-binding arm, wherein the first heavy chain comprises the amino acid sequence of SEQ ID NO: 26 and the light chain comprises the amino acid sequence of SEQ ID NO: 30. In some embodiments, the multispecific antibody comprises a second heavy chain comprising D2-HCVR and D3-HCVR of the second antigen-binding arm paired with a second light chain comprising D2-LCVR and a third light chain comprising D3-LCVR, wherein the second heavy chain comprises the amino acid sequence of SEQ ID NO: 28; the second light chain comprises the amino acid sequence of SEQ ID NO: 30, and the third light chain comprises the amino acid sequence of SEQ ID NO: 30. In some embodiments, the multispecific antibody comprises a first heavy chain comprising the HCVR of the first antigen-binding arm, wherein the first heavy chain is paired with a light chain comprising the LCVR of the first antigen-binding arm, wherein the first heavy chain comprises the amino acid sequence of SEQ ID NO: 56 and the light chain comprises the amino acid sequence of SEQ ID NO: 60. In some embodiments, the multispecific antibody comprises a second heavy chain comprising D2-HCVR and D3-HCVR of the second antigen-binding arm paired with a second light chain comprising D2-LCVR and a third light chain comprising D3-LCVR, wherein the second heavy chain comprises the amino acid sequence of SEQ ID NO: 58; the second light chain comprises the amino acid sequence of SEQ ID NO: 60, and the third light chain comprises the amino acid sequence of SEQ ID NO: 60.

In one aspect, the disclosed technology relates to pharmaceutical composition comprising a multispecific antigen-binding molecule that binds specifically to PSMA and 4-1BB, and a pharmaceutically acceptable carrier.

In one aspect, the disclosed technology relates to an isolated nucleic acid molecule. In one embodiment the isolated nucleic acid molecule comprises a polynucleotide sequence encoding an amino acid sequence comprising D1-HCVR of an multispecific antigen-binding molecule that binds specifically to PSMA and 4-1BB. In one embodiment the isolated nucleic acid molecule comprises a nucleic acid sequence encoding an amino acid sequence comprising D2-HCVR and D3-HCVR of a multispecific antigen-binding molecule that binds specifically to PSMA and 4-1BB. In one embodiment the isolated nucleic acid molecule comprises a nucleic acid sequence encoding an amino acid sequence comprising one or more LCVRs of a group consisting of D1-LCVR, D2-LCVR, and D3-LCVR of a multispecific antigen-binding molecule that binds specifically to PSMA and 4-1BB. In one embodiment an expression vector contains an isolated nucleic acid molecule comprising a nucleic acid sequence encoding an amino acid sequence comprising D1-HCVR, D2-HCVR, D3-HCVR, D1-LCVR, D2-LCVR and/or D3-LCVR of a multispecific antigen-binding molecule that binds specifically to PSMA and 4-1BB. In one embodiment a set of expression vectors contains a set of nucleic acid molecules comprising nucleic acid sequences encoding amino acid sequences comprising D1-HCVR, D2-HCVR, D3-HCVR, D1-LCVR, D2-LCVR and/or D3-LCVR of a multispecific antigen-binding molecule that binds specifically to PSMA and 4-1BB. In one embodiment expression vector or set of expression vectors is contained within a host cell. In one embodiment the host cell is E. coli or CHO cell.

In one aspect, the disclosed technology relates to a method of producing a multispecific antigen-binding molecule that binds specifically to PSMA and 4-1BB, the method comprising growing the host cell under conditions permitting production of the multispecific antigen-binding molecule, wherein the host cell comprises a first nucleic acid molecule comprising a nucleic acid sequence encoding an amino acid sequence comprising a first antigen-binding arm comprising a heavy chain variable region (HCVR) of the multispecific antigen-binding molecule, a second nucleic acid molecule comprising a nucleic acid sequence encoding a second amino acid sequence comprising a second antigen-binding arm comprising heavy chain variable regions (HCVRs) of the multispecific antigen-binding molecule, and a third nucleic acid molecule comprising a nucleic acid sequence encoding an amino acid sequence comprising a common light chain variable region (LCVR). In one embodiment, the host cell comprises a first nucleic acid molecule comprising a nucleic acid sequence encoding an amino acid sequence comprising a heavy chain of a first antigen-binding arm of a multispecific antigen-binding molecule, a second nucleic acid molecule comprising a nucleic acid sequence encoding an amino acid sequence comprising a heavy chain of a second antigen-binding arm of a multispecific antigen-binding molecule, and a third nucleic acid molecule comprising a nucleic acid sequence encoding an amino acid sequence comprising a common light chain.

In one aspect, the disclosed technology relates to a method of inhibiting growth of a tumor in a subject, comprising administering a multispecific antigen-binding molecule that binds specifically to PSMA and 4-1BB to the subject. In one embodiment, the tumor is a prostate cancer tumor. In one embodiment, the method comprises administering the multispecific antigen-binding molecule to the subject, wherein the tumor is selected from the group consisting of prostate cancer, hepatocellular carcinoma, non-small cell lung cancer, melanoma, pancreatic ductal adenocarcinoma, glioma, or breast cancer, or another cancer characterized in part by having PSMA+ cells. In one embodiment, the method further comprises administering an additional therapeutic agent or therapeutic regimen. In one embodiment, the additional therapeutic is an anti-PSMA x anti-CD3 multispecific antigen-binding molecule. In one embodiment, the additional therapeutic agent or therapeutic regimen comprises a chemotherapeutic drug, a DNA alkylator, an immunomodulator, a proteasome inhibitor, a histone deacetylase inhibitor, radiotherapy, a stem cell transplant, surgery, a different multispecific antibody that interacts with a different tumor cell surface antigen and a T cell or immune cell antigen, an antibody drug conjugate, a multispecific antibody conjugated to an anti-tumor agent, a checkpoint inhibitor, a PD-1 inhibitor, a PD-L1 inhibitor, a CTLA-4 inhibitor, a CD28 agonist, a 4-1BB agonist, a radioligand, hormone therapy, a CD3 blocker, a STEAP1 inhibitor, an EGFR inhibitor, a GITR agonist, a cancer vaccine, an oncolytic virus, an immunocytokine, IL4 inhibitor, IL6 inhibitor, IL2 or modified IL2, IL12, 1110, IL23, a T cell comprising a chimeric antigen receptor (CAR-T cell), or combinations thereof.

In one embodiment, the subject has a PSMA+ tumor. In one embodiment, the additional therapeutic agent is a PD-1 inhibitor. In one embodiment, the PD-1 inhibitor is an anti-PD-1 antibody or antigen-binding fragment thereof, or an anti-PD-L1 antibody or antigen-binding fragment thereof. In one embodiment, the anti-PD-1 inhibitor is selected from cemiplimab, nivolumab, pembrolizumab, durvalumab, atezolizumab, and avelumab. In one embodiment, the PD-1 inhibitor is cemiplimab.

In one aspect, the disclosed technology relates to the use of a multispecific antigen-binding molecule in the treatment of a disease or disorder associated with expression of PSMA. In some embodiments, the disease or disorder is cancer. In some embodiments, the cancer is prostate cancer, hepatocellular carcinoma, non-small cell lung cancer, melanoma, pancreatic ductal adenocarcinoma, glioma, breast cancer, or another cancer characterized in part by having PSMA+ cells. In some embodiments, the multispecific antigen-binding molecule or pharmaceutical composition is for use in combination with an anti-PD-1 antibody or antigen-binding fragment thereof. In some embodiments, the multispecific antigen-binding molecule or pharmaceutical composition is injected intravenously, intramuscularly, or subcutaneously.

### BRIEF DESCRIPTION OF THE DRAWINGS

FIG. 1 is a diagram showing the structure of an embodiment of the multispecific antigen-binding molecule provided herein.

FIGS. 2A-2D relate to Example 3. FIG. 2A is a graph showing the binding of the indicated antibodies to CD16−/CD2+/CD4+ human T cells. FIG. 2B is a graph showing binding of the indicated antibodies to CD16−/CD2+/CD8+ human T cells. FIG. 2C is a graph showing the binding of the indicated antibodies to CD16−/CD2+/CD4+ cynomolgous T cells. FIG. 2D is a graph showing the binding of the indicated antibodies to CD16−/CD2+/CD8+ cynomolgous T cells.

FIGS. 3A-3C relate to Example 3. FIG. 3A is a graph showing the binding of the indicated antibodies to C4-2 cells. FIG. 3B is a graph showing the binding of the indicated antibodies to 2RV1 cells. FIG. 3C is a graph showing the binding of the indicated antibodies to Raji cells.

FIGS. 4A-4C relate to Example 4. FIG. 4A is a graph showing the % viability of C4-1 cells treated with the indicated antibodies. FIG. 4B is a graph showing the % of T cells that are CD4+ and CD25+ when treated with the indicated antibodies. FIG. 4C is a graph showing the % of T cells that are CD8+ and CD25+ when treated with the indicated antibodies.

FIG. 5 relates to Example 6 and is a graph showing IL2 release by human primary T cells upon treatment with the indicated antibodies in the presence of DU-145/hPSMA cells.

FIG. 6 relates to Example 6 and is a graph showing IFNγ release by human primary T cells upon treatment with the indicated antibodies in the presence of DU-145/hPSMA cells.

FIGS. 7A-7B relate to Example 7. FIG. 7A is a graph showing average tumor volume over time in mice administered the indicated antibodies at the indicated doses. FIG. 7B is a graph showing average tumor volume over time in mice administered the indicated antibodies at the indicated doses. Doses are indicated in the parentheses following the name of each antibody and are in milligrams per kilogram of body weight (mg/kg). Error bars represent+/−SEM. Statistical significance determined with 2-way ANOVA and uncorrected Fisher's LSD comparison test (**, p<0.01; *, p<0.05).

FIGS. 8A-8B relate to Example 7. FIG. 8A is a graph showing the % of mice surviving at each time point when administered the indicated antibodies at the indicated doses. FIG. 8B is a graph showing the % of mice surviving at each time point when administered the indicated antibodies at the indicated doses. Doses are indicated in the parentheses following the name of each antibody and are in milligrams per kilogram of body weight (mg/kg).

FIGS. 9A-9F relate to Example 7. FIG. 9A is a graph showing tumor volume over time of individual mice administered REGN15505 at 0.05 mg/kg. FIG. 9B is a graph showing tumor volume over time of individual mice administered REGN15505 at 0.5 mg/kg. FIG. 9C is a graph showing tumor volume over time of individual mice administered REGN15505 at 5 mg/kg. FIG. 9D is a graph showing tumor volume over time of individual mice administered anti-PD-1 and REGN15505 at 0.05 mg/kg. FIG. 9E is a graph showing tumor volume over time of individual mice administered anti-PD-1 and REGN15505 at 0.5 mg/kg. FIG. 9F is a graph showing tumor volume over time of individual mice administered anti-PD-1 and REGN15505 at 5 mg/kg.

FIGS. 10A-10E relate to Example 7. FIG. 10A is a graph showing tumor volume over time of individual mice administered multispecific antibody-matched isotype control at 5 mg/kg. FIG. 10B is a graph showing tumor volume over time of individual mice administered multispecific 4-1BB antibody control at 5 mg/kg. FIG. 10C is a graph showing tumor volume over time of individual mice administered anti-PD-1 and multispecific antibody-matched isotype control at 5 mg/kg. FIG. 10D is a graph showing tumor volume over time of individual mice administered anti-PD-1 and multispecific 4-1BB antibody control at 5 mg/kg. FIG. 10E is a graph showing tumor volume over time of individual mice administered anti-PD-1 and bivalent 4-1BB antibody at 5 mg/kg.

FIGS. 11A-11J relate to Example 7. FIG. 11A is a graph showing IFNγ release upon treatment with the indicated antibodies at the indicated doses. FIG. 11B is a graph showing IL10 release upon treatment with the indicated antibodies at the indicated doses. FIG. 11C is a graph showing IL2 release upon treatment with the indicated antibodies at the indicated doses. FIG. 11D is a graph showing TNFα release upon treatment with the indicated antibodies at the indicated doses. FIG. 11E is a graph showing IL6 release upon treatment with the indicated antibodies at the indicated doses. FIG. 11F is a graph showing IL5 release upon treatment with the indicated antibodies at the indicated doses. FIG. 11G is a graph showing IL4 release upon treatment with the indicated antibodies at the indicated doses. FIG. 11H is a graph showing KCGRO release upon treatment with the indicated antibodies at the indicated doses. FIG. 11I is a graph showing IL-1B release upon treatment with the indicated antibodies at the indicated doses. FIG. 11J is a graph showing IL12p70 release upon treatment with the indicated antibodies at the indicated doses. Doses are indicated in the parentheses following the name of each antibody and are in milligrams per kilogram of body weight (mg/kg).

FIGS. 12A-12D relate to Example 8. FIG. 12A is a graph showing average tumor volume over time of mice administered the indicated antibodies prior to implantation with a second tumor. Error bars represent+/−SEM. FIG. 12B is a graph showing tumor volume of a second tumor over time of individual mice administered isotype control prior to implantation of the second tumor. FIG. 12C is a graph showing tumor volume of a second tumor over time of individual mice administered anti-PD-1 and REGN15505 prior to implantation of the second tumor. FIG. 12D is a graph showing tumor volume of a second tumor over time of individual mice administered anti-PD-1 and REGN15505 prior to implantation of the second tumor with CD8 depletion during second tumor growth.

FIGS. 13A-13B relate to Example 9. FIG. 13A is a graph showing average tumor volume over time in mice administered the indicated antibodies at the indicated doses. FIG. 13 B is a graph showing the % of mice surviving at each time point when administered the indicated antibodies at the indicated doses. “AgxCD3” refers to unrelated antigen x CD3. Doses are indicated in the parentheses following the name of each antibody and are in milligrams per kilogram of body weight (mg/kg).

FIGS. 14A-14F relate to Example 9. FIG. 14A is a graph showing tumor volume over time of individual mice administered AgxCD3 antibody at 5 mg/kg and isotype control at 5 mg/kg. FIG. 14B is a graph showing tumor volume over time of individual mice administered PSMAxCD3 antibody at 5 mg/kg and isotype control at 5 mg/kg. FIG. 14C is a graph showing tumor volume over time of individual mice administered PSMAxCD3 antibody at 0.02 mg/kg and isotype control at 5 mg/kg. FIG. 14D is a graph showing tumor volume over time of individual mice administered AgxCD3 antibody at 5 mg/kg and REGN15505 at 5 mg/kg. FIG. 14E is a graph showing tumor volume over time of individual mice administered PSMAxCD3 antibody at 0.02 mg/kg and REGN15505 at 5 mg/kg. FIG. 14F is a graph showing tumor volume over time of individual mice administered PSMAxCD3 antibody at 0.02 mg/kg and bivalent 4-1BB antibody at 5 mg/kg. “AgxCD3” refers to unrelated antigen x CD3. Doses are indicated in the parentheses following the name of each antibody and are in milligrams per kilogram of body weight (mg/kg).

FIGS. 15A-15J relate to Example 9 and are graphs showing serum cytokine concentration on Day 0. FIG. 15A is a graph showing IFNγ concentration upon treatment with the indicated antibodies at the indicated doses. FIG. 15B is a graph showing IL-2 concentration upon treatment with the indicated antibodies at the indicated doses. FIG. 15C is a graph showing IL-4 concentration upon treatment with the indicated antibodies at the indicated doses. FIG. 15D is a graph showing IL-5 concentration upon treatment with the indicated antibodies at the indicated doses. FIG. 15E is a graph showing IL-6 concentration upon treatment with the indicated antibodies at the indicated doses. FIG. 15F is a graph showing TNFα concentration upon treatment with the indicated antibodies at the indicated doses. FIG. 15G is a graph showing IL-10 concentration upon treatment with the indicated antibodies at the indicated doses. FIG. 15H is a graph showing KCGRO concentration upon treatment with the indicated antibodies at the indicated doses. FIG. 15I is a graph showing IL-1B concentration upon treatment with the indicated antibodies at the indicated doses. FIG. 15J is a graph showing IL-12p70 concentration upon treatment with the indicated antibodies at the indicated doses. “AgxCD3” refers to unrelated antigen x CD3. Doses are indicated in the parentheses following the name of each antibody and are in milligrams per kilogram of body weight (mg/kg). All measurements are from samples taken on Day 0.

FIGS. 16A-16J relate to Example 9 and are graphs showing serum cytokine concentration on Day 7. FIG. 16A is a graph showing IFNγ concentration upon treatment with the indicated antibodies at the indicated doses. FIG. 16B is a graph showing IL-2 concentration upon treatment with the indicated antibodies at the indicated doses. FIG. 16C is a graph showing IL-4 concentration upon treatment with the indicated antibodies at the indicated doses.

FIG. 16D is a graph showing IL-5 concentration upon treatment with the indicated antibodies at the indicated doses. FIG. 16E is a graph showing IL-6 concentration upon treatment with the indicated antibodies at the indicated doses. FIG. 16F is a graph showing TNFα concentration upon treatment with the indicated antibodies at the indicated doses. FIG. 16G is a graph showing IL-10 concentration upon treatment with the indicated antibodies at the indicated doses. FIG. 16H is a graph showing KCGRO concentration upon treatment with the indicated antibodies at the indicated doses. FIG. 16I is a graph showing IL-1ß concentration upon treatment with the indicated antibodies at the indicated doses. FIG. 16J is a graph showing IL-12p70 concentration upon treatment with the indicated antibodies at the indicated doses. “AgxCD3” refers to unrelated antigen x CD3. Doses are indicated in the parentheses following the name of each antibody and are in milligrams per kilogram of body weight (mg/kg). All measurements are from samples taken on Day 7.

FIGS. 17A-17J relate to Example 9 and are graphs showing serum cytokine concentration on Day 14. FIG. 17A is a graph showing IFNγ concentration upon treatment with the indicated antibodies at the indicated doses. FIG. 17B is a graph showing IL-2 concentration upon treatment with the indicated antibodies at the indicated doses. FIG. 17C is a graph showing IL-4 concentration upon treatment with the indicated antibodies at the indicated doses. FIG. 17D is a graph showing IL-5 concentration upon treatment with the indicated antibodies at the indicated doses. FIG. 17E is a graph showing IL-6 concentration upon treatment with the indicated antibodies at the indicated doses. FIG. 17F is a graph showing TNFα concentration upon treatment with the indicated antibodies at the indicated doses. FIG. 17G is a graph showing IL-10 concentration upon treatment with the indicated antibodies at the indicated doses. FIG. 17H is a graph showing KCGRO concentration upon treatment with the indicated antibodies at the indicated doses. FIG. 17I is a graph showing IL-1ß concentration upon treatment with the indicated antibodies at the indicated doses. FIG. 17J is a graph showing IL-12p70 concentration upon treatment with the indicated antibodies at the indicated doses. “AgxCD3” refers to unrelated antigen x CD3. Doses are indicated in the parentheses following the name of each antibody and are in milligrams per kilogram of body weight (mg/kg). All measurements are from samples taken on Day 14.

### DETAILED DESCRIPTION

The present disclosure relates, in part, to multispecific antigen-binding molecules that specifically bind prostate-specific membrane antigen (PSMA, also known as FOLH1) and 4-1BB (also known as CD137 and TNFRSF9) and their use in treating various diseases, including cancer. The multispecific antigen-binding molecules disclosed herein can be used alone or in combination with other agents for treating cancers that express PSMA.

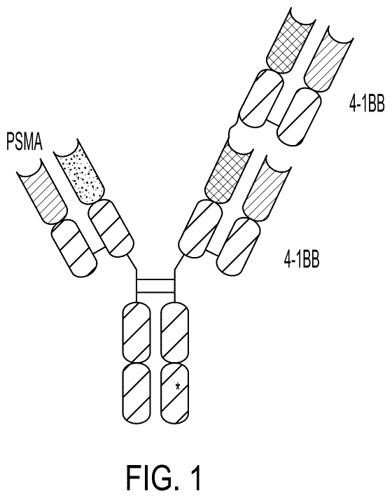

The multispecific antigen-binding molecules disclosed herein contain three antigen-binding domains, D1, D2, and D3. The D1 domain specifically binds PSMA. The D2 domain and the D3 domain each specifically bind 4-1BB. In some aspects, D2 and D3 bind different 4-1BB epitopes. In some aspects, D2 and D3 bind the same 4-1BB epitopes. In some aspects, the multispecific antigen-binding molecules provided herein contain two antigen-binding arms, A1 and A2; A1 contains D1, and A2 contains D2 and D3. In some aspects, D2 is linked to D3 via a linker, forming stacked antigen-binding domains on the A2 arm. The combination of the A1 arm and the stacked A2 arm is termed a 1+2 format. The antigen-binding domains of A2 may be contained in Fabs, an “inner” Fab2 and an “outer” Fab3. In some embodiments, D2 is contained in Fab2 and D3 is contained in Fab3. The Fab2 and Fab3 of the anti-PSMA x anti-4-1BB 1+2 antigen-binding protein are connected via a linker from the N-terminus of the V_{H}-2 of Fab2 to the C-terminus of the C_{H}1-3 of Fab 3. See FIG. 1. In some aspects, the amino acid sequences of D2 and D3 heavy chain variable regions (HCVR) are identical or substantially similar, i.e., less than 5, or less than 4, or less than 3 amino acid differences in a HCVR or a heavy chain complementarity determining region (HCDR). In some aspects, the amino acid sequences of D2 and D3 HCVRs are identical or substantially similar, i.e., 2 or 1 amino acid differences in a HCVR or a HCDR. In some aspects, the D2 and D3 HCVRs have different antigen-binding sequences.

The present disclosure provides multispecific antigen-binding molecules that bind PSMA and 4-1BB. Such multispecific antigen-binding molecules are also referred to herein as “anti-PSMA x anti-4-1BB multispecific antigen-binding molecules”. The PSMA antigen-binding arm, A1, contains one antigen-binding domain, D1. The 4-1BB antigen-binding arm, A2, comprises two antigen-binding domains, D2 and D3. D2 is referred to herein as the 4-1BB “inner” binding domain, and D3 is referred to herein as the 4-1BB “outer” binding domain. The multispecific antigen-binding molecules having the stacked antigen-binding domains are referred to herein as anti-PSMA x anti-4-1BB 1+2 multispecific antigen-binding molecules, PSMAx4-1BB 1+2 multispecific antigen-binding molecules, or anti-PSMA/anti-4-1BB 1+2 multispecific antigen-binding molecules.

The anti-PSMA portion of the anti-PSMA x anti-4-1BB multispecific molecule is useful for targeting tumor cells that express PSMA (e.g., prostate cancer cells), and the anti-4-1BB portion of the multispecific molecule is useful for providing co-stimulation of T cells activated by cognate MHC peptide or tumor targeted CD3 multispecific antigen-binding molecules. The simultaneous binding of PSMA on a tumor cell and 4-1BB on a T cell facilitates directed killing (cell lysis) of the targeted tumor cell by the activated T cell. The anti-PSMA x anti-4-1BB 1+2 multispecific molecules provided herein are therefore useful, inter alia, for treating diseases and disorders related to or caused by PSMA-expressing tumors (e.g., prostate cancer).

The multispecific antigen-binding molecules provided herein contain a first antigen-binding arm, A1, containing an antigen-binding domain, D1, that specifically binds human PSMA, and a second antigen-binding arm, A2, containing two antigen-binding domains, D2 and D3, that specifically bind 4-1BB. The present disclosure includes anti-PSMA x anti-4-1BB 1+2 multispecific molecules (e.g., multispecific antigen-binding molecules) wherein each antigen-binding domain contains a heavy chain variable region (HCVR) paired with a light chain variable region (LCVR). In certain exemplary embodiments of the present disclosure, the anti-PSMA antigen-binding domain and the anti-4-1BB antigen-binding domains each contain different, distinct HCVRs paired with a common LCVR. In certain exemplary embodiments, the common LCVR is a universal LCVR. For example, as illustrated in Example 1 herein, multispecific antigen-binding molecules were constructed containing a first antigen-binding domain that specifically binds PSMA, where the first antigen-binding domain (D1) contains an HCVR derived from an anti-PSMA antigen-binding molecule; and a second antigen-binding domain (D2) and a third antigen-binding domain (D3) that specifically bind 4-1BB, where the second antigen-binding domain (D2) and third antigen-binding domain (D2) each contain a HCVR derived from an anti-4-1BB antigen-binding molecule, where each HCVR is paired with a universal light chain LCVR. In such embodiments, the first, second, and third antigen-binding domains comprise distinct anti-PSMA and anti-4-1BB HCVRs, but share a common light chain LCVR.

The present disclosure provides multispecific antigen-binding molecules including multispecific antibodies comprising a first antigen-binding arm, which includes one antigen-domain that specifically binds prostate-specific membrane antigen (PSMA, also known as FOLH1), and a second antigen-binding arm, which includes two antigen-binding domains that each bind 4-1BB (also known as CD137 and tumor necrosis factor receptor superfamily 9 (TNFRSF9)), (“PSMAx4-1BB” or “4-1BBxPSMA” antibodies) that enhance T-cell activation, provide robust inhibition of tumor growth, and promote survival in subjects with cancer, low levels of systemic cytokine secretion, induce long lived anti-tumor immunity, and exhibit low levels of systemic cytokine secretion. The multispecific antibodies of the present disclosure provide an approach to exploit PSMA expression in the tumor to anchor the 4-1BB agonist antigen-binding fragment intratumorally, which provides a co-stimulatory signal to T cells in a spatially restricted manner. The PSMAx4-1BB antibodies of the present disclosure mimick the natural ligand of 4-1BB, by bridging PSMA+ target cells with T cells expressing 4-1BB receptor. Therefore, in the presence of PSMA+ target cells, PSMAx4-1BB 1+2 multispecific antibodies provide signal 2 in order to enhance the activation of T cells in the presence of a signal 1.

In certain embodiments, the multispecific antigen-binding molecules enhance the T cell mediated killing of tumor cells expressing PSMA, when used in combination with a PD-1 inhibitor.

In one aspect, the present disclosure provides an isolated multispecific antigen-binding molecule including a first antigen-binding domain (D1) that specifically binds prostate-specific membrane antigen (PSMA), a second antigen-binding domain (D2) that specifically binds 4-1BB, and a third antigen-binding domain (D3) that specifically binds 4-1BB.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present disclosure, the preferred methods and materials are now described.

### Definitions

All references to proteins, polypeptides and protein fragments herein are intended to refer to the human version of the respective protein, polypeptide or protein fragment unless explicitly specified as being from a non-human species. Thus, the expression, “PSMA” means human PSMA unless specified as being from a non-human species (e.g., “mouse PSMA,” “monkey PSMA,” etc.). In an embodiment of the disclosure, human PSMA comprises the amino acid sequence set forth in NCBI accession no. Q04609. In one embodiment, a human PSMA fragment is shown with a C-terminal myc-myc-hexahistidine tag (hPSMA.mmH). The expression, “4-1BB” means human 4-1BB unless specified as being from a non-human species (e.g., “mouse 4-1BB,” “monkey 4-1BB,” etc.). In an embodiment of the disclosure, human 4-1BB comprises the amino acid sequence as set forth NCBI accession No. AAA53133.1. In one embodiment, a human 4-1BB fragment is expressed with a C-terminal myc-myc-hexahistidine tag (hCD137 (4-1BB) mmH).

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antigen-binding proteins (e.g., antibodies or antigen-binding fragments).

The following references relate to BLAST algorithms often used for sequence analysis: BLAST ALGORITHMS: Altschul et al. (2005) FEBS J. 272 (20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

The term “antigen-binding molecule”, as used herein, means any antigen-binding molecule or molecular complex comprising at least one complementarity determining region (CDR) that specifically binds to or interacts with a particular antigen (e.g., PSMA or 4-1BB). The term “antigen-binding molecule” includes immunoglobulin molecules comprising four polypeptide chains, two heavy chains (HC) and two or three light chains (LC) inter-connected by disulfide bonds, as well as multimers thereof (e.g., IgM). The term “antigen-binding molecule” includes immunoglobulin molecules having two antigen-binding arms, A1 and A2. The term “antigen-binding molecule” also includes immunoglobulin molecules consisting of five polypeptide chains, two heavy chains (HC) and two or three light chains (LC) inter-connected by disulfide bonds. Each antigen-binding arm comprises a heavy chain (abbreviated herein as HCVR, VH, or V_{H}), which in turn comprises at least one heavy chain variable region, and a light chain, which in turn comprises a light chain variable region (abbreviated herein as LCVR, VL, or V_{L}).

An “antibody” is an immunoglobulin molecule comprising four polypeptide chains, two heavy chains (HC) and two light chains (LC) inter-connected by disulfide bonds. In the context of the present disclosure, the term includes an immunoglobulin molecule with five polypeptide chains, two heavy chains and three light chains. Each heavy chain (HC) comprises a heavy chain variable region (abbreviated herein as HCVR, VH, or V_{H}) and a heavy chain constant region (abbreviated herein as CH or C_{H}) (e.g., IgG, IgG1 or IgG4). The heavy chain constant region comprises three domains, C_{H}1, C_{H}2 and C_{H}3. Each light chain (LC) comprises a light chain variable region (abbreviated herein as LCVR, VL, or V_{L}) and a light chain constant region (e.g., lambda or kappa). The light chain constant region comprises one domain (C_{L}1). The V_{H} and V_{L} regions can be further subdivided into regions of hypervariability, termed complementarity determining regions (CDRs), interspersed with regions that are less conserved, termed framework regions (FR). Each V_{H} and V_{L} includes three CDRs and four FRs, arranged from amino-terminus to carboxy-terminus in the following order: FR1, CDR1, FR2, CDR2, FR3, CDR3, FR4. A heavy chain CDR may be referred to as HCDR and a light chain CDR may be referred to as LCDR. In different embodiments of the disclosure, the FRs of an antibody (or antigen-binding fragment thereof) may be identical to the human germline sequences, or may be naturally or artificially modified.

An antigen-binding arm of a Y-shaped IgG antibody (e.g., a PSMA or 4-1BB or binding arm) refers to a structural portion of the antibody that confers binding specificity to the antigen. For example, an antigen-binding arm of an IgG antibody has a heavy chain (HC) associated with one or more light chains (LC).

An antibody which, for example, is multispecific includes an arm (including one or more antigen-binding domains) that binds to a first antigen and another arm (including one or more antigen-binding domains) that binds to a second antigen. For example, a PSMAx4-1BB multispecific antibody includes one arm that binds PSMA and another arm that binds to 4-1BB.

Multispecific antigen-binding molecules (e.g., multispecific antibodies) may have a targeting arm that binds to a first antigen and an effector arm that binds to second antigen. The targeting arm may include a first antigen-binding domain that binds to an antigen on target cells (e.g., tumor cells). The effector arm may include a second antigen-binding domain (e.g., anti-4-1BB) and a third antigen-binging domain (e.g., anti-4-1BB) that binds to the antigens on effector cells (e.g., T cells). In the context of the present disclosure, the targeting arm binds to the tumor associated antigen (TAA) PSMA, and the effector arm binds to 4-1BB.

An “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. A multispecific antigen-binding fragment of an antibody binds to multiple antigens (e.g., two different antigens). Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments.

An antigen-binding fragment of an antibody will, in an embodiment of the disclosure, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

Illustratively, FIG. 1 shows an A1 antigen-binding arm that comprises Fab1, which is specific for PSMA, and an A2 antigen-binding arm that comprises two Fabs, “inner” Fab2 and “outer” Fab3, each of which are specific to 4-1BB. The Fab1, Fab2, and Fab3 light chains can be universal light chains or cognate light chains. In this example, the V_{L}-1 is linked to the C_{L}-1, which is linked to the C_{H}1-1 on the A1 arm. On the A2 arm, the V_{L}-3 is linked to the C_{L}-3, which is linked to the C_{H}1-3. Likewise, the V_{L}-2 is linked to the C_{L}-2, which is linked to the C_{H}1-2. The Fab2 and Fab3 of the anti-PSMA x anti-4-1BB 1+2 construct are connected via a linker from the N-terminus of the V_{H}-2 inner Fab2 to the C-terminus of the C_{H}1-3 outer Fab3.

The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library. The present disclosure includes recombinant antigen-binding proteins as set forth herein.

The term “specifically binds” or “binds specifically” refers to those antigen-binding proteins (e.g., antibodies or antigen-binding fragments thereof) having a binding affinity to an antigen, such as PSMA or 4-1BB protein, expressed as K_{D}, of less than about 10⁻⁶ M (e.g., 10⁻⁷ M, 10⁻⁸ M, 10⁻⁹ M, 10⁻¹⁰ M, 10⁻¹¹ M or 10⁻¹² M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. “Anti-PSMA” refers to an antigen-binding protein (or other molecule such as an antigen-binding arm), for example an antibody or antigen-binding fragment thereof, that binds specifically to PSMA and “anti-4-1BB” refers to an antigen-binding protein (or other molecule such as an antigen-binding arm), for example an antibody or antigen-binding fragment thereof, that binds specifically to 4-1BB. “PSMAx4-1BB” or “PSMAx4-1BB” refers to refers to an antigen-binding protein (or other molecule), for example an antibody or antigen-binding fragment thereof, that binds specifically to PSMA and to 4-1BB.

The present disclosure includes antigen-binding proteins, e.g., antibodies or antigen-binding fragments, that bind to the same PSMA and 4-1BB epitopes as an antigen-binding protein of the present disclosure (e.g., REGN15505 and REGN15510).

The term “epitope” refers to an antigenic determinant (e.g., on PSMA or 4-1BB) that interacts with a specific antigen-binding site of an antigen-binding protein, e.g., a variable region of an antibody molecule, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Methods for determining the epitope of an antigen-binding protein, e.g., antibody or fragment or polypeptide, include alanine scanning mutational analysis, peptide blot analysis (Reineke (2004) Methods Mol. Biol. 248:443-63), peptide cleavage analysis, crystallographic studies and NMR analysis. In addition, methods such as epitope excision, epitope extraction and chemical modification of antigens can be employed (Tomer (2000) Prot. Sci. 9:487-496). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267:252-259; Engen and Smith (2001) Anal. Chem. 73: 256A-265A.

The present disclosure includes antigen-binding proteins that compete for binding to PSMA and 4-1BB with an antigen-binding protein of the present disclosure (e.g., REGN15505 and REGN15510). The term “competes” as used herein, refers to an antigen-binding protein (e.g., antibody or antigen-binding fragment thereof) that binds to an antigen and inhibits or blocks the binding of another antigen-binding protein (e.g., antibody or antigen-binding fragment thereof) to the antigen. Unless otherwise stated, the term also includes competition between two antigen-binding proteins e.g., antibodies, in both orientations, i.e., a first antibody that binds antigen and blocks binding by a second antibody and vice versa. Thus, in an embodiment of the disclosure, competition occurs in one such orientation. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody (e.g., via steric hindrance). Competition between antigen-binding proteins (e.g., antibodies) may be measured by methods known in the art, for example, by a real-time, label-free bio-layer interferometry assay. Also, binding competition between antigen-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment of the disclosure which is modified in some way retains the ability to specifically bind to PSMA and 4-1BB, e.g., retains at least 10% of its PSMA and 4-1BB binding activity (when compared to the parental antibody) when that activity is expressed on a molar basis. Preferably, an antibody or antigen-binding fragment of the disclosure retains at least 20%, 50%, 70%, 80%, 90%, 95% or 100% or more of the PSMA and 4-1BB binding affinity as the parental antibody. It is also intended that an antibody or antigen-binding fragment of the disclosure may include conservative or non-conservative amino acid substitutions (referred to as “conservative variants” or “function conserved variants” of the antibody) that do not substantially alter its biologic activity.

A “variant” of a polypeptide, such as an immunoglobulin chain (e.g., a chain of REGN15505 or REGN15510) V_{H}, V_{L}, HC or LC or CDR thereof comprising the amino acid sequence specifically set forth herein), refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5 or 99.9%) identical or similar to a referenced amino acid sequence that is set forth herein (e.g., any of SEQ ID NOs: 2, 4, 6; 8; 10; 12; 14; 16; 18; 20; 22; 24; 26; 28; 30; 32; 34; 36; 38; 40; 42; 44; 46; 48; 50; 52; 54; 56; 58; 60); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Moreover, a variant of a polypeptide may include a polypeptide such as an immunoglobulin chain (e.g., a chain of REGN15505 or REGN15510) V_{H}, V_{L}, HC or LC or CDR thereof) which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. For example, the present disclosure includes PSMAx4-1BB antigen-binding proteins which include a PSMA binding arm immunoglobulin light chain (or V_{L}) variant comprising the amino acid sequence set forth in SEQ ID NO: 18 but having one or more of such mutations and/or an immunoglobulin heavy chain (or V_{H}) variant comprising the amino acid sequence set forth in SEQ ID NO: 2 or SEQ ID NO: 32 but having one or more of such mutations. In an embodiment of the disclosure, a PSMAx4-1BB antigen-binding protein includes an immunoglobulin light chain variant comprising LCDR1, LCDR2 and LCDR3, wherein one or more (e.g., 1 or 2 or 3) of such CDRs has one or more of such mutations (e.g., conservative substitutions) and/or an immunoglobulin heavy chain variant comprising HCDR1, HCDR2 and HCDR3 of an “inner” V_{H} and HCDR1, HCDR2, and HCDR3 of an “outer” V_{H}, wherein one or more (e.g., 1 or 2 or 3) of such CDRs has one or more of such mutations (e.g., conservative substitutions).

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity. The present disclosure includes PSMAx4-1BB antigen-binding proteins and/or binding arms comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Alternatively, a conservative replacement is any change having a positive value in the PAM250 log-likelihood matrix disclosed in Gonnet et al. (1992) Science 256:1443-45.

As used herein, the singular forms “a,” “an,” and “the” include plural reference unless the context clearly dictates otherwise. As used herein, the terms “including,” “comprising,” “containing,” or “having” and variations thereof are meant to encompass the items listed thereafter and equivalents thereof as well as additional subject matter unless otherwise noted. As used herein, the phrases “in one embodiment,” “in various embodiments,” “in some embodiments,” and the like are used repeatedly. Such phrases do not necessarily refer to the same embodiment, but they may unless the context dictates otherwise. As used herein, the terms “and/or” or “/” means any one of the items, any combination of the items, or all of the items with which this term is associated.

### Multispecific PSMAx4-1BB Antigen-Binding Molecules, Antibodies and Antigen-Binding Fragments Thereof

The present disclosure includes multispecific antigen-binding molecules that specifically bind 4-1BB and PSMA. Such molecules may be referred to herein as, e.g., “anti-PSMA x anti-4-1BB 1+2” or “anti-PSMA/anti-4-1BB 1+2,” or “anti-PSMAx4-1BB 1+2” or “PSMA x4-1BB 1+2” multispecific molecules, or other similar terminology.

The present disclosure includes multispecific antigen-binding molecules wherein one arm A2 of the immunoglobulin has two antigen-binding domains which bind human 4-1BB, a 4-1BB “inner” domain and a 4-1BB “outer” domain, and the other arm A1 of the immunoglobulin is specific for binding human PSMA. The 4-1BB-binding arm can comprise any of the HCVR/LCVR or CDR amino acid sequences as set forth in Table 3 herein, in a stacked format.

The anti-PSMA portion of the anti-PSMA x anti-4-1BB multispecific molecule is useful for targeting tumor cells that express PSMA (e.g., prostate cancer cells), and the anti-4-1BB portion of the multispecific molecule is useful for providing co-stimulation of T cells activated by cognate MHC peptide or tumor targeted CD3 multispecific antigen-binding molecules. The simultaneous binding of PSMA on a tumor cell and 4-1BB on a T cell facilitates directed killing (cell lysis) of the targeted tumor cell by the activated T cell. The anti-PSMA x anti-4-1BB 1+2 multispecific molecules provided herein are therefore useful, inter alia, for treating diseases and disorders related to or caused by PSMA-expressing tumors (e.g., prostate cancer).

In certain embodiments, the 4-1BB-binding arm binds to human 4-1BB and facilitates human T cell activation. In certain embodiments, the 4-1BB-binding arm binds to human 4-1BB and induces human T cell activation. In other embodiments, the 4-1BB-binding arm binds to human 4-1BB and induces tumor-associated antigen-expressing cell killing in the context of a multispecific or multispecific antigen-binding molecule. The PSMA-binding arm can comprise any of the HCVR/LCVR or CDR amino acid sequences as set forth in Table 1 herein.

As used herein, the expression “antigen-binding molecule” means a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen-binding molecule is an antigen-binding molecule or a fragment of an antigen-binding molecule, as those terms are defined elsewhere herein.

As used herein, the expression “multispecific antigen-binding molecule” means a protein, polypeptide or molecular complex comprising at least a first antigen-binding domain and a second antigen-binding domain. Each antigen-binding domain within the multispecific antigen-binding molecule comprises at least one CDR that alone, or in combination with one or more additional CDRs and/or FRs, specifically binds to a particular antigen. In the context of the present disclosure, the first antigen-binding arm A1 specifically binds a first antigen (e.g., PSMA), and the second antigen-binding arm A2 specifically binds a second and third antigen (e.g., 4-1BB), distinct from the first antigen.

The multispecific antigen-binding molecules discussed herein can comprise a human IgG heavy chain constant region. In some cases, the human IgG heavy chain constant region is isotype IgG1. In some cases, the human IgG heavy chain constant region is isotype IgG4. In various embodiments, the multispecific antigen-binding molecule comprises a chimeric hinge that reduces Fcγ receptor binding relative to a wild-type hinge of the same isotype.

The first antigen-binding arm A1 and the second antigen-binding arm A2 may be directly or indirectly connected to one another to form a multispecific antigen-binding molecule of the present disclosure. Alternatively, the first antigen-binding arm A1 and the second antigen-binding arm A2 may each be connected to a separate multimerizing domain. The association of one multimerizing domain with another multimerizing domain facilitates the association between the two antigen-binding domains, thereby forming a multispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Multispecific antigen-binding molecules of the present disclosure will typically comprise two multimerizing domains, e.g., two Fc domains that are each individually part of a separate antigen-binding molecule heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any multispecific antigen-binding molecule format or technology may be used to make the multispecific antigen-binding molecules of the present disclosure. For example, an antigen-binding molecule or fragment thereof having a first antigen-binding specificity can be functionally linked (e.g., by chemical coupling, genetic fusion, noncovalent association or otherwise) to one or more other molecular entities, such as another antigen-binding molecule or antigen-binding molecule fragment having a second antigen-binding specificity to produce a multispecific antigen-binding molecule. Specific exemplary multispecific formats that can be used in the context of the present disclosure include, without limitation, e.g., scFv-based or diabody multispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² multispecific formats (see, e.g., Klein et al. 2012, mAbs 4:6, 1-11, and references cited therein, for a review of the foregoing formats).

In the context of multispecific antigen-binding molecules of the present disclosure, the multimerizing domains, e.g., Fc domains, may comprise one or more amino acid changes (e.g., insertions, deletions or substitutions) as compared to the wild-type, naturally occurring version of the Fc domain. For example, the disclosure includes multispecific antigen-binding molecules comprising one or more modifications in the Fc domain that results in a modified Fc domain having a modified binding interaction (e.g., enhanced or diminished) between Fc and FcRn. In one embodiment, the multispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

The present disclosure also includes multispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the multispecific antigen-binding molecule to Protein A as compared to a bi-specific antigen-binding molecule lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). The second C_{H}3 may further comprise a L105P modification (by IMGT; L455P by EU) See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antigen-binding molecules; N44S, K52N, and V82I (IMGT; N384S, K392N, and V422I by EU) in the case of IgG2 antigen-binding molecules; and Q15R, N44S, K52N, V57M, R69K, E79Q, and V82I (by IMGT; Q355R, N384S, K392N, V397M, R409K, E419Q, and V422I by EU) in the case of IgG4 antigen-binding molecules.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric Fc domains that can be included in any of the antigen-binding molecules of the present disclosure are described in U.S. Pat. No. 9,359,437, which is herein incorporated in its entirety. Chimeric Fc domains having these general structural arrangements, and variants thereof, can have altered Fc receptor binding, which in turn affects Fc effector function.

Antibodies and antigen-binding fragments of the present disclosure comprise immunoglobulin chains including the amino acid sequences specifically set forth herein (and variants thereof) as well as cellular and in vitro post-translational modifications to the antibody or fragment. For example, the present disclosure includes antibodies and antigen-binding fragments thereof that specifically bind to PSMA and 4-1BB comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

### Polynucleotides and Methods of Making

An isolated polynucleotide molecule or a set of polynucleotide molecules comprising polynucleotide sequences encoding the immunoglobulin chains of any PSMAx4-1BB multispecific antigen-binding molecule set forth herein forms part of the present disclosure. The present disclosure also includes a vector or a set of vectors comprising the polynucleotide molecules and/or a host cell (e.g., Chinese hamster ovary (CHO) cell) comprising the polynucleotide molecules, vector(s) or antigen-binding protein set forth herein.

A polynucleotide molecule or sequence includes DNA and RNA. The present disclosure includes any polynucleotide molecule or sequence of the present disclosure, for example, encoding an immunoglobulin V_{H}, V_{L}, CDR-H, CDR-L, HC or LC of a PSMA Binding Arm and/or a 4-1BB Binding Arm, optionally, which is operably linked to a promoter or other expression control sequence. For example, the present disclosure provides any polynucleotide (e.g., DNA) that includes a nucleotide sequence set forth in Table 2 and a nucleotide sequence set forth in Table 4.

The present disclosure includes a polynucleotide comprising a nucleotide sequence set forth in SEQ ID NO: 1, 3, 5, 7, 9, 11, 13, 15, 17, 19, 21, 29, 31, 33, 35, 39, 41, 43, 45, 49, 51, 53, 55, 57, 59, 61, 62, 63, 64, 65, 66, 67, and/or 68, optionally operably linked to a promoter or other expression control sequence or other polynucleotide sequence.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the disclosure. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (VIIIa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

The present disclosure includes polynucleotides encoding immunoglobulin polypeptide chains which are variants of those whose nucleotide sequence is specifically set forth herein. A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein; when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear). In an embodiment of the disclosure, a variant of a nucleotide sequence specifically set forth herein comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides. Such mutations may, in an embodiment of the disclosure, be missense or nonsense mutations. In an embodiment of the disclosure, such a variant polynucleotide encodes an immunoglobulin polypeptide chain which can be incorporated into a PSMA Binding Arm and/or 4-1BB Binding Arm, i.e., such that the protein retains specific binding to PSMA and/or 4-1BB.

Eukaryotic and prokaryotic host cells, including mammalian cells, may be used as hosts for expression of a PSMAx4-1BB antigen-binding protein (e.g., antibody or antigen-binding fragment thereof) or an antigen-binding arm thereof. Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present disclosure includes an isolated host cell (e.g., a CHO cell or any type of host cell set forth above) comprising an anti-PSMA x anti-4-1BB antigen-binding protein of the present disclosure, such as REGN15505 and REGN15510 or a polynucleotide encoding an immunoglobulin (lg) heavy and/or light chain thereof); and/or one or more polynucleotides encoding the PSMA binding arm and 4-1BB binding arm of a multispecific antigen-binding protein of the present disclosure.

The present disclosure also includes a cell which is expressing PSMA and/or 4-1BB or an antigenic fragment or fusion thereof (e.g., His6, Fc and/or myc) which is bound by a PSMAx4-1BB antigen-binding protein of the present disclosure (e.g., an antibody or antigen-binding fragment thereof), for example, REGN15505 and REGN15510, as well as multispecific antibodies prepared by combining any of the PSMA heavy chain variable region (HCVR) arms of Table 1 (e.g., HCVR arms of the parental monoclonal antibodies 11838P2 and 11453N) with any of the 4-1BB HCVR arms of Table 3 (e.g., HCVR arms of the parental 25923 and 35333P2), for example, wherein the cell is in the body of a subject or is in vitro.

In addition, the present disclosure also provides a complex comprising a PSMAx4-1BB, antigen-binding protein of the present disclosure, e.g., antibody or antigen-binding fragment thereof, as discussed herein complexed with PSMA and/or 4-1BB polypeptide or an antigenic fragment thereof or fusion thereof and/or with a secondary antibody or antigen-binding fragment thereof (e.g., detectably labeled secondary antibody) that binds specifically to the PSMAx4-1BB antibody or fragment. In an embodiment of the disclosure, the complex is in vitro (e.g., is immobilized to a solid substrate) or is in the body of a subject. In an embodiment of the disclosure, the PSMA is on the surface of a tumor cell or antigen presenting cell and the 4-1BB is on the surface of an immune cell, e.g., a T cell. In an embodiment of the disclosure, the T cell is activated.

There are several methods by which to produce recombinant antibodies which are known in the art. One example of a method for recombinant production of antibodies is disclosed in U.S. Pat. No. 4,816,567. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

The present disclosure includes recombinant methods for making an anti-PSMA x anti-4-1BB (e.g., REGN15505 and REGN15510) antigen-binding protein of the present disclosure, such as an antibody or antigen-binding fragment thereof of the present disclosure, or an immunoglobulin chain thereof, comprising
(i) introducing, into a host cell, one or more polynucleotides encoding the light and heavy immunoglobulin chains encoding the PSMAx4-1BB antigen-binding protein's antigen-binding arms for example, wherein the polynucleotide is in a vector; and/or integrates into the host cell chromosome and/or is operably linked to a promoter;
(ii) culturing the host cell (e.g., CHO or Pichia or Pichia pastoris) under conditions favorable to expression of the polynucleotide and,
(iii) optionally, isolating the antigen-binding protein (e.g., antibody or antigen-binding fragment) or chain from the host cell and/or medium in which the host cell is grown. The present disclosure also includes PSMAx4-1BB antigen-binding proteins, such as antibodies and antigen-binding fragments thereof, which are the product of the production methods set forth herein, and, optionally, the purification methods set forth herein.

In an embodiment of the disclosure, a method for making a PSMAx4-1BB (e.g., REGN15505 and REGN15510) antigen-binding protein, e.g., antibody or antigen-binding fragment thereof, includes a method of purifying the antigen-binding protein, e.g., by column chromatography, precipitation and/or filtration. As discussed, the product of such a method also forms part of the present disclosure.

### Sequence Variants

The antibodies and multispecific antigen-binding molecules of the present disclosure may comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germ line sequences available from, for example, public antibody sequence databases. The antigen-binding molecules of the present disclosure may comprise antigen-binding fragments which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germ line sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germ line sequence while certain other residues that differ from the original germ line sequence are maintained or are mutated to the corresponding residue of a different germline sequence. Once obtained, antigen-binding domains that contain one or more germline mutations can be easily tested for one or more desired properties such as improved binding specificity, increased binding affinity, improved or enhanced antagonistic or agonistic biological properties (as the case may be), reduced immunogenicity, etc. Multispecific antigen-binding molecules comprising one or more antigen-binding domains obtained in this general manner are encompassed within the present disclosure.

The present disclosure also includes antigen-binding molecules wherein one or both antigen-binding domains comprise variants of any of the HCVR, LCVR, and/or CDR amino acid sequences disclosed herein having one or more conservative substitutions. For example, the present disclosure includes antigen-binding molecules comprising an antigen-binding domain having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445. A “moderately conservative” replacement is any change having a nonnegative value in the PAM250 log-likelihood matrix.

The present disclosure also includes antigen-binding molecules comprising an antigen-binding domain with an HCVR, LCVR, and/or CDR amino acid sequence that is substantially identical to any of the HCVR, LCVR, and/or CDR amino acid sequences disclosed herein. The term “substantial identity” or “substantially identical,” when referring to an amino acid sequence means that two amino acid sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410; Altschul et al. (1997) Nucleic Acids Res. 25:3389-402.

### Antibodies Comprising Fc Variants

According to certain embodiments of the present disclosure, anti-PSMA x anti-4-1BB multispecific antigen-binding molecules are provided comprising an Fc domain comprising one or more mutations which enhance or diminish antibody binding to the FcRn receptor, e.g., at acidic pH as compared to neutral pH. For example, the present disclosure includes antibodies and antigen-binding molecules comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position
250 (e.g., E or Q);
250 and 428 (e.g., L or F);
252 (e.g., L/Y/F/W or T),
254 (e.g., S or T), and/or
256 (e.g., S/R/Q/E/D or T);
   or a modification at position
428 and/or 433 (e.g., H/L/R/S/P/Q or K) and/or
434 (e.g., H/F or Y);
   or a modification at position
250 and/or 428;
   or a modification at position
307 or 308 (e.g., 308F, V308F), and/or
434.

In one embodiment, the modification comprises a
428L (e.g., M428L) and 434S (e.g., N434S) modification;
a 428L, 259I (e.g., V259I), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

For example, the present disclosure includes PSMAx4-1BB multispecific antigen-binding molecules comprising an Fc domain comprising one or more pairs or groups of mutations selected from the group consisting of:
250Q and 248L (e.g., T250Q and M248L);
252Y, 254T and 256E (e.g., M252Y, S254T and T256E);
428L and 434S (e.g., M428L and N434S); and
433K and 434F (e.g., H433K and N434F).

The present disclosure also includes multispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the multispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

All possible combinations of the foregoing Fc domain mutations, and other mutations within the antibody variable domains disclosed herein, are contemplated within the scope of the present disclosure.

### Biological Characteristics of the Multispecific Antibodies and Antigen-Binding Molecules

The present disclosure includes antibodies and antigen-binding fragments thereof that bind human 4-1BB and PSMA with high affinity. The present disclosure also includes antibodies and antigen-binding fragments thereof that bind human 4-1BB and/or PSMA with medium or low affinity, depending on the therapeutic context and particular targeting properties that are desired. For example, in the context of a multispecific antigen-binding molecule, wherein one arm binds 4-1BB and another arm binds a target antigen (e.g., PSMA), it may be desirable for the target antigen-binding arm to bind the target antigen with high affinity while the anti-4-1BB arm binds 4-1BB with only moderate or low affinity. In this manner, preferential targeting of the antigen-binding molecule to cells expressing the target antigen may be achieved while avoiding general/untargeted 4-1BB binding and the consequent adverse side effects associated therewith.

According to certain embodiments, the present disclosure includes antibodies and antigen-binding fragments of antibodies that bind human 4-1BB (e.g., at 25° C.) with a K_{D} of less than about 200 nM as measured by surface plasmon resonance, e.g., using an assay format as defined in Example 2 herein. In certain embodiments, the antibodies or antigen-binding fragments of the present disclosure bind 4-1BB with a Kp of less than about 200 nM, less than about 150 nM, less than about 100 nM, less than about 90 nM, less than about 80 nM, less than about 60 nM, less than about 40 nM, less than about 30 nM, less than 20 nM, less than 10 nM, or less than 5 nM as measured by surface plasmon resonance, e.g., using an assay format as defined in Example 2 herein, or a substantially similar assay. In certain embodiments, the antibodies or antigen-binding fragments of the present disclosure bind 4-1BB with a Kp of about 10 nM to about 200 nM.

The present disclosure also includes antibodies and antigen-binding fragments thereof that bind 4-1BB with a dissociative half-life (t½) of greater than about 0.1 minutes as measured by surface plasmon resonance at 25° C. or 37° C., e.g., using an assay format as defined in Example 2 herein, or a substantially similar assay. In certain embodiments, the antibodies or antigen-binding fragments of the present disclosure bind 4-1BB with a t_{1/2} of greater than about 0.5 minutes, greater than about 1 minute, greater than about 3 minutes, greater than about 5 minutes, greater than about 10 minutes, greater than about 15 minutes, greater than about 20 minutes, greater than about 30 minutes, greater than about 40 minutes, or greater than about 50 minutes, as measured by surface plasmon resonance at 25° C. or 37° C., e.g., using an assay format as defined in Example 2 herein, or a substantially similar assay.

According to certain embodiments, the present disclosure includes antibodies and antigen-binding fragments of antibodies that bind human PSMA (e.g., at 25° C.) with a K_{D} of less than about 1 nM as measured by surface plasmon resonance, e.g., using an assay format as defined in Example 2 herein. In certain embodiments, the antibodies or antigen-binding fragments of the present disclosure bind PSMA with a K_{D} of less than about 1 nM, less than about 0.9 nM, less than about 0.8 nM, less than about 0.6 nM, less than about 0.4 nM, less than about 0.3 nM, less than 0.2 nM, less than 0.1 nM, or less than 0.05 nM as measured by surface plasmon resonance, e.g., using an assay format as defined in Example 2 herein, or a substantially similar assay. In certain embodiments, the antibodies or antigen-binding fragments of the present disclosure bind PSMA with a K_{D} of about 0.05 nM to about 0.2 nM.

The present disclosure also includes antibodies and antigen-binding fragments thereof that bind PSMA with a dissociative half-life (t½) of greater than about 30 minutes as measured by surface plasmon resonance at 25° C. or 37° C., e.g., using an assay format as defined in the Examples herein, or a substantially similar assay. In certain embodiments, the antibodies or antigen-binding fragments of the present disclosure bind PSMA with a t½ of greater than about 30 minutes, greater than about 60 minutes, greater than about 120 minutes, greater than about 150 minutes, greater than about 180 minutes, or greater than about 200 minutes, as measured by surface plasmon resonance at 25° C. or 37° C., e.g., using an assay format as defined in Example 2 herein, or a substantially similar assay.

The present disclosure includes multispecific antigen-binding molecules (e.g., multispecific antibodies) which are capable of simultaneously binding to human 4-1BB and human PSMA. According to certain embodiments, the multispecific antigen-binding molecules of the disclosure specifically interact with cells that express 4-1BB and/or PSMA. The extent to which a multispecific antigen-binding molecule binds cells that express 4-1BB and/or PSMA can be assessed by fluorescence activated cell sorting (FACS), as illustrated in Example 3 herein. For example, the present disclosure includes multispecific antigen-binding molecules which specifically bind human cell lines which express 4-1BB but not PSMA (e.g., Jurkat cell genetically engineered to express 4-1BB). In some embodiments, the multispecific antigen-binding molecules bind to 4-1BB-expressing human or cynomolgus T cells with an EC₅₀ value less than 1×10⁻⁵ M. In some embodiments, the multispecific antigen-binding molecules bind to 4-1BB-expressing human or cynomolgus T cells with an EC₅₀ value of 1×10⁻¹² M to 1×10⁻⁵ M. In certain embodiments, the multispecific antigen-binding molecules bind to 4-1BB-expressing human or cynomolgus T cells with an EC₅₀ value of 1×10⁻¹² M to 1×10⁻⁹ M. In certain embodiments, the multispecific antigen-binding molecules bind to 4-1BB-expressing human or cynomolgus T cells with an EC₅₀ value of 1×10⁻¹² M to 1×10⁻¹⁰ M. In certain embodiments, the multispecific antigen-binding molecules bind to the surface of cell lines expressing PSMA with an EC₅₀ of less than about 4×10⁻⁹ M. The binding of the multispecific antigen-binding molecules to the surface of cells or cell lines can be measured by an in vitro FACS binding assay as described in Example 3.

The present disclosure includes PSMAx4-1BB multispecific antigen-binding molecules which are capable of depleting tumor cells in a subject. For example, according to certain embodiments, PSMAx4-1BB multispecific antigen-binding molecules are provided, wherein a single administration of the antigen-binding molecule to a subject at a therapeutically effective dose causes a reduction in the number of tumor cells in the subject.

The present disclosure includes anti-PSMA x anti-CD28 multispecific antigen-binding molecules which are capable of activating T cells by engaging PSMA on target cells and 4-1BB on T cells (see Example 5). For example, binding of the anti-PSMA X anti-4-1BB multispecific antigen-binding molecules to T cells can lead to an increase in IL2 release (Example 6). As such, the multispecific antigen-binding molecules of the disclosure may prove useful in promoting a T cell mediated immune response.

The present disclosure includes anti-PSMA x anti-4-1BB multispecific antigen-binding molecules which are capable of binding to PSMA expressed on cells surface. A variety of tumor cells express PSMA, including breast tumor cells (e.g., HeLa, MCF-7 and MDA-MB-231), melanoma cells (e.g., A375), lung tumor cells (e.g., HCC44), ovarian cancer cells (e.g., ES-2, SNU-8, MCAS), pancreatic cancer cells (eig., SNU-324), prostate cancer cells (e.g., LNCaP, DU145). As such, the multispecific antibodies of the disclosure may prove useful in treating a multitude of cancer indications.

The present disclosure includes anti-PSMA x anti-4-1BB multispecific antigen-binding molecules which are capable of enhancing the cytotoxic potency of anti-tumor associated antigen (TAA) x anti-CD3 multispecific antibodies across a variety of cell lines (See Example 4). In certain embodiments, the TAA is selected from the group consisting of AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD40, CDK4, CEA, CTLA4, cyclin-B1, CYP1B1, EGFR, EGFRvlll, ErbB2/Her2, ErbB3, ErbB4, ETV6-AML, EpCAM, EphA2, Fra-1, FOLR1, GAGE proteins (e.g., GAGE-1, -2), GD2, GD3, GloboH, glypican-3, GM3, gp 100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3. The anti-PSMA X anti-4-1BB antibodies may also prove useful when combined with a checkpoint inhibitor, for example, an antibody to PD-1, PD-L1, or any other checkpoint inhibitor.

Monotherapy with the antigen-binding molecules of the present disclosure showed dose-dependent anti-tumor activity (see Example 7 herein). Combination of the antigen-binding molecules with a PD-1 inhibitor (e.g., an anti-PD-1 antibody) or with a sub-efficacious dose of a bispecific PSMAxCD3 antibody resulted in greater anti-tumor activity than the antigen-binding molecule alone or than a high dose the PSMAxCD3 antibody (see Examples 7 and 9 herein). Similar trends were observed for improvement of survival. Furthermore, combination therapy with PD-1 mAb mediated long-term tumor immunity, with tumor-free animals rejecting a second tumor with no additional treatment (see Example 8). Importantly, monotherapy or combination therapy that showed significantly lowered tumor growth generally resulted in lower cytokine release than that observed for monotherapy with high-dose PSMAxCD3 or similar combination therapy regimens using bivalent 4-1BB antibody (see Example 9).

### Epitope Mapping and Related Technologies

The epitope on 4-1BB or PSMA to which the antigen-binding molecules of the present disclosure bind may consist of a single contiguous sequence of 3 or more (e.g., 3, 4, 5, 6, 7, 8, 9, 10, 11, 12, 13, 14, 15, 16, 17, 18, 19, 20 or more) amino acids of a 4-1BB protein or a PSMA protein. Alternatively, the epitope may consist of a plurality of non-contiguous amino acids (or amino acid sequences) of 4-1BB or PSMA. The antibodies of the disclosure may interact with amino acids contained within a 4-1BB monomer, or may interact with amino acids on two different 4-1BB chains of a 4-1BB dimer. The term “epitope,” as used herein, refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques that can be used to determine an epitope or binding domain of a particular antibody or antigen-binding domain include, e.g., routine crossblocking assay such as that described in Antibodies, Harlow and Lane (Cold Spring Harbor Press, Cold Spring Harb., NY), point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2): 252-259; Engen and Smith (2001) Anal. Chem. 73: 256A-265A. Alternatively, in certain embodiments, the protein of interest binds to the antibody, followed by hydrogen-deuterium exchange. After dissociation of the antibody, the target protein is subjected to protease cleavage and mass spectrometry analysis, thereby revealing the non-deuterium-labeled residues which correspond to the specific amino acids with which the antibody interacts. X-ray crystal structure analysis can also be used to identify the amino acids within a polypeptide with which an antibody interacts.

The present disclosure further includes anti-4-1BB and anti-PSMA antibodies that bind to the same epitope as any of the specific exemplary antibodies described herein (e.g., antibodies comprising any of the amino acid sequences as set forth in Tables 1, 3, 6, and 8). Likewise, the present disclosure also includes anti-4-1BB and/or anti-PSMA antibodies that compete for binding to 4-1BB and/or PSMA with any of the specific exemplary antibodies described herein (e.g., antibodies comprising any of the amino acid sequences as set forth in Tables 1, 3, 6, and 8 herein).

The present disclosure also includes multispecific antigen-binding molecules comprising a first antigen-binding domain that specifically binds human PSMA, and a second antigen-binding domain that specifically binds human 4-1BB, and a third antigen-binding domain that specifically binds human 4-1BB, wherein the first antigen-binding domain binds to the same epitope on PSMA as any of the specific exemplary PSMA-specific antigen-binding domains described herein, and/or wherein the second or third antigen-binding domain binds to the same epitope on 4-1BB as any of the specific exemplary 4-1BB-specific antigen-binding domains described herein.

Likewise, the present disclosure also includes multispecific antigen-binding molecules comprising a first antigen-binding domain that specifically binds human PSMA, a second antigen-binding domain that specifically binds human 4-1BB, and a second antigen-binding domain that specifically binds human 4-1BB, wherein the first antigen-binding domain competes for binding to PSMA with any of the specific exemplary PSMA-specific antigen-binding domains described herein, and/or wherein the second or third antigen-binding domain competes for binding to 4-1BB with any of the specific exemplary 4-1BB-specific antigen-binding domains described herein.

One can easily determine whether a particular antigen-binding molecule (e.g., antibody) or antigen-binding domain thereof binds to the same epitope as, or competes for binding with, a reference antigen-binding molecule of the present disclosure by using routine methods known in the art. For example, to determine if a test antibody binds to the same epitope on 4-1BB (or PSMA) as a reference multispecific antigen-binding molecule of the present disclosure, the reference multispecific molecule is first allowed to bind to a 4-1BB protein (or PSMA protein). Next, the ability of a test antibody to bind to the 4-1BB (or PSMA) molecule is assessed. If the test antibody is able to bind to 4-1BB (or PSMA) following saturation binding with the reference multispecific antigen-binding molecule, it can be concluded that the test antibody does not compete for binding to 4-1BB (or PSMA) with the reference multispecific antigen-binding molecule and/or that there is steric interference between antibodies that are binding different sites on the antigen. On the other hand, if the test antibody is not able to bind to the 4-1BB (or PSMA) molecule following saturation binding with the reference multispecific antigen-binding molecule, then the test antibody competes for binding to 4-1BB (or PSMA) with the reference multispecific antigen-binding molecule of the disclosure. Additional routine experimentation (e.g., peptide mutation and binding analyses) can then be carried out to confirm whether the observed lack of binding of the test antibody is in fact due to binding to the same epitope as the reference multispecific antigen-binding molecule or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antigen-binding proteins compete for binding to an antigen if, e.g., a 1-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins may bind to the same epitope if essentially all amino acid mutations in the antigen that reduce or eliminate binding of one antigen-binding protein reduce or eliminate binding of the other. Two antigen-binding proteins may have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a 4-1BB protein (or PSMA protein) under saturating conditions followed by assessment of binding of the test antibody to the 4-1BB (or PSMA) molecule. In a second orientation, the test antibody is allowed to bind to a 4-1BB (or PSMA) molecule under saturating conditions followed by assessment of binding of the reference antigen-binding molecule to the 4-1BB (or PSMA) molecule. If, in both orientations, only the first (saturating) antigen-binding molecule is capable of binding to the 4-1BB (or PSMA) molecule, then it is concluded that the test antibody and the reference antigen-binding molecule compete for binding to 4-1BB (or PSMA). As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

### Preparation of Antigen-Binding Domains and Construction of Multispecific Molecules

Antigen-binding domains specific for particular antigens can be prepared by any antigen-binding molecule generating technology known in the art. Once obtained, different antigen-binding domains provided herein, specific for two different antigens (e.g., PSMA and 4-1BB), can be appropriately arranged relative to one another to produce a multispecific antigen-binding molecule of the present disclosure using routine methods. (A discussion of exemplary multispecific antigen-binding molecule formats that can be used to construct the multispecific antigen-binding molecules of the present disclosure is provided elsewhere herein). In certain embodiments, one or more of the individual components (e.g., heavy and light chains) of the multispecific antigen-binding molecules of the present disclosure are derived from chimeric, humanized or fully human antigen-binding molecules. Methods for making such antibodies are well known in the art. For example, one or more of the heavy and/or light chains of the multispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., 4-1BB or PSMA) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the multispecific antigen-binding molecules of the present disclosure.

Genetically engineered animals may be used to make human multispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human multispecific antigen-binding molecules comprising two different heavy chains that associate with an identical light chain that comprises a variable domain derived from one of two different human light chain variable region gene segments. (See, e.g., US 2011/0195454 for a detailed discussion of such engineered mice and the use thereof to produce multispecific antigen-binding molecules). Fully human refers to an antigen-binding molecule, or antigen-binding fragment thereof, or immunoglobulin domain thereof, comprising an amino acid sequence encoded by a DNA derived from a human sequence over the entire length of each polypeptide of the antigen-binding molecule or antigen-binding fragment thereof, or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g., compared to any wild-type human immunoglobulin regions or domains.

### Bioequivalents

The present disclosure encompasses antigen-binding molecules having amino acid sequences that vary from those of the described antibodies but that retain the ability to bind 4-1BB and PSMA. Such variant molecules comprise one or more additions, deletions, or substitutions of amino acids when compared to parent sequence, but exhibit biological activity that is essentially equivalent to that of the described antigen-binding molecules. Likewise, the antigen-binding molecules-encoding DNA sequences of the present disclosure encompass sequences that comprise one or more additions, deletions, or substitutions of nucleotides when compared to the disclosed sequence, but that encode an antigen-binding molecule that is essentially bioequivalent to the described antigen-binding molecules of the disclosure. Examples of such variant amino acid and DNA sequences are discussed above.

The present disclosure includes antigen-binding molecules that are bioequivalent to any of the exemplary antigen-binding molecules set forth herein. Two antigen-binding proteins or antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the exemplary multispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include the exemplary multispecific antigen-binding molecules set forth herein comprising amino acid changes which modify the glycosylation characteristics of the antibodies, e.g., mutations which eliminate or remove glycosylation.

### Species Selectivity and Species Cross-Reactivity

The present disclosure, according to certain embodiments, provides antigen-binding molecules that bind to human 4-1BB but not to 4-1BB from other species. The present disclosure also provides antigen-binding molecules that bind to human PSMA but not to PSMA from other species. The present disclosure also includes antigen-binding molecules that bind to human 4-1BB and to 4-1BB from one or more non-human species; and/or antigen-binding molecules that bind to human PSMA and to PSMA from one or more non-human species.

According to certain exemplary embodiments of the disclosure, antigen-binding molecules are provided which bind to human 4-1BB and/or human PSMA and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee 4-1BB and or PSMA. For example, in particular exemplary embodiments of the disclosed herein, multispecific antigen-binding molecules are provided comprising a first antigen-binding arm that binds human PSMA or cynomolgus PSMA, and a second antigen-binding arm comprising a second antigen-binding domain and a third antigen-binding domain wherein the second antigen-binding arm specifically binds human 4-1BB, or multispecific antigen-binding molecules comprising a second antigen-binding arm comprising second and third antigen-binding domains that bind human 4-1BB and/or cynomolgus 4-1BB, and a first antigen-binding arm that specifically binds human PSMA.

### Immunoconjugates

The disclosure encompasses PSMAx4-1BB antigen-binding proteins, e.g., antibodies or antigen-binding fragments, such as REGN15505 and REGN15510, conjugated to another moiety, e.g., a therapeutic moiety (an “immunoconjugate”). In an embodiment of the disclosure, PSMAx4-1BB antigen-binding protein, e.g., antibody or antigen-binding fragment, is conjugated to any of the further therapeutic agents set forth herein. As used herein, the term “immunoconjugate” refers to an antigen-binding protein, e.g., an antibody or antigen-binding fragment, which is chemically or biologically linked to another antigen-binding protein, a drug, a radioactive agent, a reporter moiety, an enzyme, a peptide, a protein or a therapeutic agent.

In certain embodiments, the therapeutic moiety may be a cytotoxin, a chemotherapeutic drug, an immunosuppressant or a radioisotope. Cytotoxic agents include any agent that is detrimental to cells. Examples of suitable cytotoxic agents and chemotherapeutic agents for forming immunoconjugates are known in the art, (see for example, WO 05/103081).

### Therapeutic Uses of the Antigen-Binding Molecules

The multispecific antibodies and antigen-binding molecules of the disclosure (and therapeutic compositions comprising the same) are useful, inter alia, for treating any disease or disorder in which stimulation, activation and/or targeting of an immune response would be beneficial. In particular, the PSMAx4-1BB multispecific antigen-binding molecules of the present disclosure may be used for the treatment, prevention and/or amelioration of a hyperproliferative disease such as cancer. In certain embodiments, the present disclosure provides methods for treating cancer in a subject, comprising administering a therapeutically effective dose of PSMAx4-1BB antigen-binding molecule, e.g., REGN15505 or REGN15510.

A hyperproliferative disease, for the purposes herein, refers to a disease characterized by abnormal, excessive and/or uncontrolled cell growth, e.g., wherein the cells express PSMA. For example, hyperproliferative diseases include cancers. Exemplary cancers include, but are not limited to esophageal carcinoma, lung squamous cell carcinoma, lung adenocarcinoma, cervical squamous cell carcinoma, glioma, thyroid cancer, lung cancer (e.g., non-small cell lung cancer), colorectal cancer, colon cancer, bladder cancer, rectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, gastroesophageal cancer, (e.g., gastroesophageal adenocarcinoma), and melanoma. Accordingly, the antibodies and the multispecific antigen-binding molecules of the present disclosure can be used in treating a wide range of cancers.

Cancer characterized by solid tumor cells or cancerous blood cells may be a PSMA-expressing cancer e.g., wherein PSMA expression in the cells of the particular subject to be treated has been confirmed, includes esophageal carcinoma, lung squamous cell carcinoma, lung adenocarcinoma, cervical squamous cell carcinoma, endometrial adenocarcinoma, bladder urothelial carcinoma, lung cancer (e.g., non-small cell lung cancer), colorectal cancer, rectal cancer, endometrial cancer, skin cancer (e.g., head & neck squamous cell carcinoma), brain cancer (e.g., glioblastoma multiforme), breast cancer, gastroesophageal cancer, (e.g., gastroesophageal adenocarcinoma), prostate cancer and/or ovarian cancer.

The antigen-binding molecules of the present disclosure may also be used to treat, e.g., primary and/or metastatic tumors arising in the colon, lung, breast, ovary, kidney, prostate, and bladder (or from any cancer discussed herein).

The antigen-binding molecules of the present disclosure may be used to residual cancer in a subject. As used herein, the term “residual cancer” means the existence or persistence of one or more cancerous cells in a subject following treatment with an anti-cancer therapy.

As used herein, the term “subject” refers to a mammal (e.g., rat, mouse, cat, dog, cow, sheep, horse, goat, rabbit), preferably a human, for example, in need of prevention and/or treatment of cancer. The subject may have a cancer, be predisposed to developing such a condition, and/or would benefit from administration of a multispecific antibody or antigen-binding fragment thereof of the present disclosure. In one embodiment, the subject may have, or be at risk of developing, a hyperproliferative disease.

Methods for treating or preventing a cancer (e.g., a PSMA-expressing cancer) in a subject in need of said treatment or prevention by administering a therapeutically effective dose amount PSMAx4-1BB antigen-binding protein, in association with an additional therapeutic agent are part of the present disclosure. Additional therapeutic agents are disclosed elsewhere herein.

An “effective” or “therapeutically effective” dose of PSMAx4-1BB antigen-binding protein, e.g., antibody or antigen-binding fragment, for treating or preventing a hyperproliferative disease, such as a PSMA-expressing cancer, is the amount of the antigen-binding protein sufficient to alleviate one or more signs and/or symptoms of the disease in the treated subject, whether by inducing the regression or elimination of such signs and/or symptoms or by inhibiting the progression of such signs and/or symptoms. In an embodiment of the disclosure, a therapeutically effective dose of PSMAx4-1BB antigen-binding protein is 0.1-2000 mg. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses may be separated by 1-8 weeks.

The dose of antigen-binding molecule administered to a patient may vary depending upon the age and the size of the patient, target disease, conditions, route of administration, and the like. The preferred dose is typically calculated according to body weight or body surface area. Depending on the severity of the condition, the frequency and the duration of the treatment can be adjusted. Effective dosages and schedules for administering a multispecific antigen-binding molecule may be determined empirically; for example, patient progress can be monitored by periodic assessment, and the dose adjusted accordingly.

### Combination Therapies

The multispecific antigen-binding molecules of the present disclosure may be used in combination with one or more agents, for example, in treating a cancer in a subject. In certain embodiments, the multispecific antigen-binding molecules may be administered in combination with one or more agents, for example, a corticosteroid, to reduce or ameliorate one or more adverse side effects, e.g., cytokine storm. In certain embodiments, the multispecific antigen-binding molecules may be administered in combination with one or more therapeutic agents or therapies for enhanced efficacy in treating cancer. Exemplary additional therapeutic agents or therapies that may be combined with or administered in combination with an antigen-binding molecule of the present disclosure include, e.g., chemotherapy (e.g., anti-cancer chemotherapy, for example, paclitaxel, docetaxel, vincristine, cisplatin, carboplatin or oxaliplatin), radiation therapy, surgery, a checkpoint inhibitor, a PD-1 inhibitor (e.g., an anti-PD-1 antibody such as pembrolizumab, nivolumab, or cemiplimab), a CTLA-4 inhibitor, LAG3 inhibitor, TIM3 inhibitor, a GITR agonist, OX40 agonist, an oncolytic virus, a cancer vaccine, a CAR-T cell, a nucleic acid therapeutic, a stem cell transplant, a modified IL2, modified IL12, IL15, IL6 inhibitor (e.g., sarilumab or tocilizumab), IL4R inhibitor (e.g., dupilumab), EGFR inhibitor, Ang2 inhibitor, VEGF inhibitor, a corticosteroid, a multispecific antibody or antigen-binding fragment thereof that binds CD3 and a tumor associated antigen (TAA) (e.g., MUC16 (mucin 16), PSMA, STEAP2, EGFR, or any of the TAA disclosed herein). Exemplary multispecific antibodies comprising an antigen-binding domain that binds CD3 include, but are not limited to those described in, e.g., WO2017/053856A1, WO2014/047231A1, WO2018/067331A1 and WO2018/058001A1.

The additional agents may be administered just prior to, concurrent with, or shortly after the administration of an antigen-binding molecule of the present disclosure; (for purposes of the present disclosure, such an administration regimen is considered the administration of an antigen-binding molecule “in combination with” an additional agent or therapeutic agent or therapy).

### Pharmaceutical Formulations and Administration

The present disclosure provides compositions that include PSMAx4-1BB antigen-binding proteins and one or more ingredients; as well as methods of use thereof and methods of making such compositions. Pharmaceutical formulations (e.g., aqueous pharmaceutical formulations that include water) comprising a PSMAx4-1BB antigen-binding protein or the present disclosure and a pharmaceutically acceptable carrier or excipient are part of the present disclosure.

The pharmaceutical compositions of the disclosure can be formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

To prepare pharmaceutical formulations of the PSMAx4-1BB antigen-binding proteins, e.g., antibodies and antigen-binding fragments thereof (e.g., REGN15505 and REGN15510), the antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, NY; Gennaro (2000) Remington: The Science and Practice of Pharmacy, Lippincott, Williams, and Wilkins, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, NY. In an embodiment of the disclosure, the pharmaceutical formulation is sterile. Such compositions are part of the present disclosure.

Pharmaceutical formulations of the present disclosure include a PSMAx4-1BB antigen-binding protein and a pharmaceutically acceptable carrier including, for example, water, buffering agents, preservatives and/or detergents.

The scope of the present disclosure includes desiccated, e.g., freeze-dried compositions, comprising a PSMAx4-1BB antigen-binding protein, e.g., antibody or antigen-binding fragment thereof, or a pharmaceutical formulation thereof that includes a pharmaceutically acceptable carrier but substantially lacks water.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, reactal, intestinal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

As discussed herein, the present disclosure provides a vessel (e.g., a plastic or glass vial) or injection device (e.g., syringe, pre-filled syringe or autoinjector) comprising any of the PSMAx4-1BB antigen-binding proteins herein, e.g., antibodies or antigen-binding fragments thereof, or a pharmaceutical formulation comprising a pharmaceutically acceptable carrier or excipient thereof.

A pharmaceutical composition of the present disclosure can be delivered subcutaneously or intravenously with a standard needle and syringe. In addition, with respect to subcutaneous delivery, a pen delivery device as known in the art, may be used in delivering a pharmaceutical composition of the present disclosure. Such a pen delivery device can be reusable or disposable.

Numerous reusable and disposable pens and autoinjector delivery devices have applications in the subcutaneous delivery of a pharmaceutical composition of the present disclosure. See e.g., AUTOPEN™ (Owen Mumford, Inc., Woodstock, UK) or the HUMIRA™ Pen (Abbott Labs, Abbott Park, IL)

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline and other isotonic solutions which may be used in combination with an appropriate solubilizing agent. Injectable oily mediums are also part of the present disclosure. Such oily mediums may be combined with a solubilizing agent.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 0.1 to about 2000 mg per dosage form in a unit dose; especially in the form of injection.

### Diagnostic Uses

The multispecific antibodies of the present disclosure may also be used to detect and/or measure 4-1BB or PSMA, or 4-1BB-expressing or PSMA-expressing cells in a sample, e.g., for diagnostic purposes. For example, PSMAx4-1BB antibody or antigen-binding fragment thereof, may be used to diagnose a condition or disease characterized by aberrant expression (e.g., over-expression, under-expression, lack of expression, etc.) of 4-1BB or PSMA. Exemplary diagnostic assays for 4-1BB or PSMA may comprise, e.g., contacting a sample, obtained from a patient, with an antibody of the disclosure, wherein the antibody is labeled with a detectable label or reporter molecule. Alternatively, an unlabeled antibody can be used in diagnostic applications in combination with a secondary antibody which is itself detectably labeled. The detectable label or reporter molecule can be a radioisotope, such as ³H, ¹⁴C, ³²p, ³⁵S, or ¹²⁵I; a fluorescent or chemiluminescent moiety such as fluorescein isothiocyanate, or rhodamine; or an enzyme such as alkaline phosphatase, betagalactosidase, horseradish peroxidase, or luciferase. Specific exemplary assays that can be used to detect or measure 4-1BB or PSMA in a sample include enzyme-linked immunosorbent assay (ELISA), radioimmunoassay (RIA), and fluorescence-activated cell sorting (FACS). Samples that can be used in 4-1BB or PSMA diagnostic assays according to the present disclosure include any tissue or fluid sample obtainable from a patient which contains detectable quantities of 4-1BB or PSMA protein, or fragments thereof, under normal or pathological conditions. Generally, levels of 4-1BB or PSMA in a particular sample obtained from a healthy patient (e.g., a patient not afflicted with a disease or condition associated with abnormal 4-1BB or PSMA levels or activity) will be measured to initially establish a baseline, or standard, level of 4-1BB or PSMA. This baseline level of 4-1BB or PSMA can then be compared against the levels of 4-1BB or PSMA measured in samples obtained from individuals suspected of having a 4-1BB or PSMA related disease or condition.

### EXAMPLES

The following examples are put forth so as to provide those of ordinary skill in the art with a complete disclosure and description of how to make and use the methods and compositions of the disclosure, and are not intended to limit the scope of what the inventors regard as their invention.

### Example 1: Construction of PSMAx4-1BB Multispecific Antibodies

This example relates to the construction of multispecific anti-PSMA x anti-4-1BB 1+2 format antibodies, “multispecific PSMAx4-1BB antibodies”. The multispecific antibodies created in accordance with the present example comprise three separate antigen-binding domains. The first antigen-binding arm comprises an antigen-binding domain that comprises a heavy chain variable region (HCVR) derived from a “parental” PSMA antibody (“VH-1”). The second antigen-binding arm comprises a second antigen-binding domain that comprises a HCVR derived from a parental anti-4-1BB antibody (“VH-2”) and a third antigen-binding domain that comprises a HCVR derived from a parental anti-4-1BB antibody (“VH-3”). Both the PSMA binding domain and the two anti-4-1BB binding domains share a common light chain. The multispecific format creates antigen-binding domains that specifically recognize PSMA (e.g., on tumor cells) and 4-1BB (e.g., on T cells).

### Generation of Anti-PSMA Antibodies

Parental, bivalent anti-PSMA antibodies were obtained by immunizing a genetically engineered mouse comprising DNA encoding human immunoglobulin heavy and universal light chain variable regions with a human PSMA antigen.

The antibodies were characterized and selected for desirable characteristics including affinity, selectivity, etc. The antibodies may have a desired constant region, for example, wild-type or modified hIgG1 or hIgG4 constant region. As will be appreciated by a person of skill in the art, an antibody with a particular constant region (e.g., modified hIgG1) may be converted to an antibody with a different constant region (e.g., modified hIgG4). While the constant region may vary according to specific use, high-affinity antigen-binding and target specificity characteristics reside in the variable region.

Table 1 sets forth the amino acid sequence identifiers of the heavy and light chain variable regions and CDRs of selected anti-PSMA antibodies of the disclosure. The corresponding nucleic acid sequence identifiers are set forth in Table 2.

TABLE 1
Amino Acid Sequence Identifiers for Selected
Parental PSMA Monoclonal Antibodies

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| 11838P2 | 2 | 4 | 6 | 8 | 18 | 20 | GAS | 24 |
| 11453N | 32 | 34 | 36 | 38 | 48 | 50 | AAS | 54 |

TABLE 2
Nucleic Acid Sequence Identifiers for Selected Parental PSMA Monoclonal Antibodies

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| 11838P2 | 1 | 3 | 5 | 7 | 17 | 19 | ggggcaagt | 23 |
| 11453N | 31 | 33 | 35 | 37 | 47 | 49 | gctgcatcc | 53 |

### Generation of Anti-4-1BB Antibodies

Parental, bivalent anti-4-1BB antibodies were obtained by immunizing a genetically engineered mouse comprising DNA encoding human immunoglobulin heavy and universal light chain variable regions with human 4-1BB protein fused to the Fc portion of mouse IgG2a, or with DNA encoding 4-1BB.

The antibody immune response was monitored by a 4-1BB-specific immunoassay. When a desired immune response was achieved, anti-4-1BB antibodies were isolated directly from antigen-positive B cells, as described in U.S. Pat. No. 7,582,298.

The antibodies were characterized and selected for desirable characteristics including affinity, selectivity, etc. The antibodies may have a desired constant region, for example, wild-type or modified hIgG1 or hIgG4 constant region. As will be appreciated by a person of skill in the art, an antibody with a particular constant region (e.g., modified hIgG1) may be converted to an antibody with a different constant region (e.g., modified hIgG4). While the constant region may vary according to specific use, high-affinity antigen-binding and target specificity characteristics reside in the variable region.

Table 3 sets forth the amino acid sequence identifiers of the heavy and light chain variable regions and CDRs of selected anti-4-1BB antibodies of the disclosure. The corresponding nucleic acid sequence identifiers are set forth in Table 4.

TABLE 3
Amino Acid Sequence Identifiers for Selected
Parental 4-1BB Monoclonal Antibodies

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| 25923 | 10 | 12 | 14 | 16 | 18 | 20 | GAS | 24 |
| 35333P2 | 40 | 42 | 44 | 46 | 48 | 50 | AAS | 54 |

TABLE 4
Nucleic Acid Sequence Identifiers for Selected Parental 4-1BB Antibodies

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| 25923 | 9 | 11 | 13 | 15 | 17 | 19 | ggggcaagt | 23 |
| 35333P2 | 39 | 41 | 43 | 45 | 47 | 49 | gctgcatcc | 53 |

Generation of Multispecific Antibodies that Bind PSMA and 4-1BB

The multispecific antigen-binding molecules that bind PSMA and 4-1BB are also referred to herein as “anti-PSMA x anti-4-1BB 1+2” or “anti-PSMA x anti-4-1BB multispecific molecules”, or “anti-PSMA/anti-4-1BB 1+2”, or “anti-PSMA/anti-4-1BB multispecific molecules”, or “PSMAx4-1BB 1+2”, or “PSMAx4-1BB multispecific molecules”. The anti-PSMA portion of the anti-PSMA x anti-4-1BB multispecific molecule is useful for targeting tumor cells that express PSMA, and the anti-4-1BB portion of the multispecific molecule is useful for targeting or activating 4-1BB-expressing cells (e.g., T cells).

An individual 4-1BB-binding Fab (i.e., a heavy chain variable region with a heavy chain CH1 domain and a light chain) binding to 4-1BB epitope 1 (ep1) or epitope 2 (ep2) were fused to the N-terminus of a 4-1BB VH domain from an existing IgG-like multispecific antibody targeting both PSMA and 4-1BB.

In exemplified multispecific antibodies, the light chain comprising the LCVR for each of the antigen-binding domains (anti-PSMA and anti-4-1BB) has an identical amino acid sequence (“common light chain”).

Mammalian expression vectors for individual heavy chains were created by InFusion Cloning (Takara Bio USA Inc.) following protocols provided by Takara Bio USA Inc. A PSMA heavy chain variable region (VH-1) was cloned into a heavy chain expression plasmid (CH1-1_CH2_CH3). A 4-1BB heavy chain variable region (VH-3, “outer VH”) was fused to a CH1 domain (CH1-3, “outer CH1”) with a linker followed by another 4-1BB heavy chain variable region (VH-2, “inner VH”) and cloned into a heavy chain expression plasmid (CH1-2_CH2_CH3(*)) containing the star mutation (H435R, Y436F, EU numbering).

Recombinant PSMA x 4-1BB 1+2 N-Fab multispecific antigen-binding molecules (msABM) were produced in CHO cells after transfection with 3 expression plasmids (i) PSMA heavy chain plasmid (ii) 4-1BB+4-1BB heavy chain plasmid (iii) a universal light chain plasmid. Stably transfected CHO cells were used to produce the PSMA x 4-1BB 1+2 N-Fab msABM, which were subsequently purified as described previously (Sci Rep. 2015 Dec. 11; 5:17943).

Table 5 summarizes the component parts of selected multispecific PSMAx4-1BB antibodies of the disclosure. Tables 6 and 7 set forth the amino acid and nucleic acid identifiers, respectively, of the selected multispecific antibodies. Table 8 shows the full-length heavy chain and light chain sequences of the selected multispecific antibodies.

TABLE 5
Summary of Component Parts of Selected Multispecific
Anti-PSMA × Anti-4-1BB Antibodies

| | D1 | D2 “inner” | D3 “outer” | |
|---|---|---|---|---|
| | Anti-PSMA | Anti-4-1BB | Anti-4-1BB | |
| Multispecific | Antigen- | Antigen- | Antigen- | Common Light |
| Antibody | Binding | Binding | Binding | Chain Variable |
| Designation | Domain | Domain | Domain | Region |
| bsAb15505 | 11838P2 | 25923 | 25923 | 1-39(PP) GL |
| bsAb15510 | 11453N | 35333P2 | 35333P2 | 1-39(PP) GL |

TABLE 6
Amino Acid Sequence Identifiers of Selected Multispecific Anti-PSMA × Anti-4-1BB Antibodies

| Multispecific | D1 (Fab1) Anti-PSMA | | | | D2 (Fab2) “Inner” Anti-4-1BB | | | |
|---|---|---|---|---|---|---|---|---|
| Antibody | Antigen-Binding Domain | | | | Antigen-Binding Domain | | | |
| Designation | VH-1 | D1-HCDR1 | D1-HCDR2 | D1-HCDR3 | VH-2 | D2-HCDR1 | D2-HCDR2 | D2-HCDR3 |
| bsAb15505 | 2 | 4 | 6 | 8 | 10 | 12 | 14 | 16 |
| bsAb15510 | 32 | 34 | 36 | 38 | 40 | 42 | 44 | 46 |

| Multispecific | D3 (Fab3) “Outer” Anti-4-1BB | | | | Common Light Chain | | | |
|---|---|---|---|---|---|---|---|---|
| Antibody | Antigen-Binding Domain | | | | Variable Region | | | |
| Designation | VH-3 | D3-HCDR1 | D3-HCDR2 | D3-HCDR3 | LCVR | LCDR1 | LCDR2 | LCDR3 |
| bsAb15505 | 10 | 12 | 14 | 16 | 18 | 20 | GAS | 24 |
| bsAb15510 | 40 | 42 | 44 | 46 | 48 | 50 | AAS | 54 |

TABLE 7
Nucleic Acid Sequence Identifiers of Selected Multispecific Anti-PSMA × Anti-4-1BB Antibodies

| Multispecific | D1 (Fab1) Anti-PSMA | | | | D2 (Fab2) “Inner” Anti-4-1BB | | | |
|---|---|---|---|---|---|---|---|---|
| Antibody | Antigen-Binding Domain | | | | Antigen-Binding Domain | | | |
| Designation | VH-1 | D1-HCDR1 | D1-HCDR2 | D1-HCDR3 | VH-2 | D2-HCDR1 | D2-HCDR2 | D2-HCDR3 |
| bsAb15505 | 1 | 3 | 5 | 7 | 61 | 62 | 63 | 64 |
| bsAb15510 | 31 | 33 | 35 | 37 | 39 | 41 | 43 | 45 |

| Multispecific | D3 (Fab3) “Outer” Anti-4-1BB | | | | Common Light Chain | | | |
|---|---|---|---|---|---|---|---|---|
| Antibody | Antigen-Binding Domain | | | | Variable Region | | | |
| Designation | VH-3 | D3-HCDR1 | D3-HCDR2 | D3-HCDR3 | LCVR | LCDR1 | LCDR2 | LCDR3 |
| bsAb15505 | 9 | 11 | 13 | 15 | 17 | 19 | ggggcaagt | 23 |
| bsAb15510 | 65 | 66 | 67 | 8 | 47 | 49 | gctgcatcc | 53 |

TABLE 8
Amino Acid and Nucleic Acid Sequence Identifiers of Heavy Chains and Light
Chains of Selected Multispecific Anti-PSMA × Anti-4-1BB Antibodies

| Multispecific | | Amino Acid SEQ ID NOs: | | | Nucleic Acid SEQ ID NOs: | | |
|---|---|---|---|---|---|---|---|
| Antibody | Antibody | Anti- | Anti-4- | Common | Anti- | Anti-4- | Common |
| Designation | Identifier | PSMA HC | 1BB HC | LC | PSMA HC | 1BB HC | LC |
| bsAb15505 | REGN15505 | 26 | 28 | 30 | 25 | 27 | 29 |
| bsAb15510 | REGN15510 | 56 | 58 | 60 | 55 | 57 | 59 |

Additional multispecific antibodies comprising a HCVR from one parental PSMA antibody and two HCVRs from a single or two different parental 4-1BB antibodies may be made using the techniques described herein. The parental PSMA antibodies used to generate these additional anti-PSMA x anti-4-1BB multispecific antibodies have HCVR sequences described above in Table 1. The 4-1BB parental antibodies used to generate these additional anti-PSMA x anti-4-1BB multispecific antibodies have the amino acid sequences described above in Table 3.

The multispecific antibodies described in the following examples consist of antigen-binding arms that bind to human PSMA and human 4-1BB protein (see Biacore binding data below). Exemplified multispecific antibodies contain a modified (chimeric) IgG4 Fc domain as set forth in U.S. Pat. No. 9,359,437.

### Example 2: Strong Binding of Multispecific PSMAx4-1BB Antibodies by Surface Plasmon Resonance

This example relates to in vitro studies demonstrating the binding characteristics of multispecific anti-PSMA x anti-4-1BB 1+2 format antibodies, “multispecific PSMAx4-1BB antibodies”, binding to dimeric human, cynomolgus, and murine PSMA; monomeric human, cynomolgus, and murine 4-1BB; and dimeric human 4-1BB proteins in an antibody capture format at 25° C.

Equilibrium dissociation constants (K_{D} values) of the multispecific PSMAx4-1BB antibodies 15505 and 15510 binding to human, cynomolgus, and murine PSMA and 4-1BB were determined using real-time surface plasmon resonance biosensor technology on a Biacore T-200 instrument. The dimeric human, cynomolgus, and murine PSMA were expressed with a N-terminal hexahistidine tag (6H.hPSMA, R&D Systems; 6H.mfPSMA, Acro Biosystems; 6H.mPSMA, R&D Systems); monomeric human, cynomolgus, and murine 4-1BB were expressed with a C-terminal myc-myc-hexahistidine tag (h4-1BB.mmH, mf4-1BB.mmH; and m4-1BB.mmH); and dimeric human 4-1BB expressed with a C-terminal murine Fc tag (h4-1BB.mFc).

The CM5 Biacore sensor surface was derivatized by amine coupling with a monoclonal mouse anti-human Fc monoclonal antibody. All Biacore binding studies were performed in a buffer composed of 10 mM HEPES PH 7.4, 150 mM NaCl, 1 mM Ca2+, 0.5 mM Mg2+, 0.05% v/v Surfactant P20 (HBS-P++ running buffer). Different concentrations of dimeric PSMA proteins (6H.hPSMA, 6H.mfPSMA, or 6H.mPSMA) ranging from 5 nM to 20 nM in 2-fold serial dilutions for 6H.hPSMA or 6H.mfPSMA; a single 20 nM solution for 6H.mPSMA, monomeric 4-1BB proteins (h4-1BB.mmH, mf4-1BB.mmH, or m4-1BB.mmH) ranging from 30 to 270 nM in 3-fold serial dilution; or dimeric human 4-1BB protein (h4-1BB.mFc) ranging from 10 to 90 nM in 3-fold serial dilution were injected over the captured multispecific PSMAx4-1BB antibodies at a flow rate of 50 μL/minute. Antibody-ligand association was monitored for 5 minutes, and dissociation was monitored for 10 minutes. At the end of each cycle, the multispecific PSMAx4-1BB antibody capture surface was regenerated using a 12 second injection of 20 mM phosphoric acid. All binding kinetics experiments were performed at 25° C.

The specific SPR-Biacore sensorgrams were obtained by a double referencing procedure. The double referencing was performed by first subtracting the signal of each injection over a reference surface (anti-hFc) from the signal over the experimental surface (anti-hFc-captured multispecific PSMAx4-1BB 1+2 antibodies) thereby removing contributions from refractive index changes. In addition, running buffer injections were performed to allow subtraction of the signal changes resulting from the dissociation of captured 1+2 format antibodies from the coupled anti-hFc surface. Kinetic association (ka) and dissociation (ka) rate constants were determined by fitting the real-time sensorgrams to a 1:1 binding model using Scrubber v2.0c curve fitting software. Binding dissociation equilibrium constants (K_{D}) and dissociative half-lives (t½) were calculated from the kinetic rate constants as:

$${K}_{D} ( M ) = {k}_{d} / {k}_{a} , and t \frac{1}{2} ( min ) = Ln ( 2 ) / 60 \cdot {k}_{d}$$

Results: The multispecific anti-PSMA x anti-4-1BB 1+2 format antibodies REGN15505 and 15510 display outstanding PSMA and 4-1BB binding characteristics. The kinetic and equilibrium binding parameters for dimeric human, cynomolgus, and mouse PSMA are set forth in Tables 9 through 11, respectively. The kinetic and equilibrium binding parameters for monomeric human, cynomolgus, and mouse 4-1BB kinetics are set forth in Tables 12 through 14, respectively. The kinetic and equilibrium binding parameters for dimeric human 4-1BB are set forth in Table 15. All results were determined at 25° C.

TABLE 9
Kinetic and Equilibrium Binding Parameters of Dimeric Human PSMA to Surface-
captured Multispecific PSMA × 4-1BB 1 + 2 Format Antibodies

| Multispecific | mAb | 6H.hPSMA | | | | |
|---|---|---|---|---|---|---|
| Antibody | Capture | Bound at | kₐ | k_{d} | K_{D} | t½ |
| Designation | (RU) | 20 nM (RU) | (1/Ms) | (1/s) | (M) | (min) |
| REGN15505 | 172.9 ± 0.5 | 31.8 | 4.66E+05 | 6.68E−05 | 1.43E−10 | 173.0 |
| REGN15510 | 147.5 ± 0.4 | 20.6 | 5.97E+05 | 1.68E−04 | 2.82E−10 | 68.6 |

TABLE 10
Kinetic and Equilibrium Binding Parameters of Dimeric Cynomolgus PSMA
to Surface-captured Multispecific PSMA × 4-1BB 1 + 2 Format Antibodies

| Multispecific | mAb | 6H.mfPSMA | | | | |
|---|---|---|---|---|---|---|
| Antibody | Capture | Bound at | kₐ | k_{d} | K_{D} | t½ |
| Designation | (RU) | 20 nM (RU) | (1/Ms) | (1/s) | (M) | (min) |
| REGN15505 | 170.8 ± 0.6 | 92.0 | 7.94E+05 | 1.42E−04 | 1.79E−10 | 81.5 |
| REGN15510 | 145.7 ± 0.7 | 66.7 | 7.29E+05 | 2.21E−04 | 3.03E−10 | 52.2 |

TABLE 11
Lack of Dimeric Murine PSMA Binding to
Surface-captured Multispecific PSMA ×
4-1BB 1 + 2 Format Antibodies

| Multispecific | mAb | 6H.mPSMA | | | | |
|---|---|---|---|---|---|---|
| Antibody | Capture | Bound at | kₐ | k_{d} | K_{D} | t½ |
| Designation | (RU) | 20 nM (RU) | (1/Ms) | (1/s) | (M) | (min) |
| REGN15505 | 169.0 | 3.2 | NB* | NB* | NB* | NB* |
| REGN15510 | 144.2 | 3.4 | NB* | NB* | NB* | NB* |

*NB = Non-binding

TABLE 12
Kinetic and Equilibrium Binding Parameters of Monomeric Human 4-1BB to Surface-
captured Multispecific PSMA × 4-1BB 1 + 2 Format Antibodies

| Multispecific | mAb | h4-1BB.mmH | | | | |
|---|---|---|---|---|---|---|
| Antibody | Capture | Bound at | kₐ | k_{d} | K_{D} | t½ |
| Designation | (RU) | 270 nM (RU) | (1/Ms) | (1/s) | (M) | (min) |
| REGN15505 | 165.9 ± 2.4 | 31.0 | 1.69E+05 | 2.00E−03 | 1.19E−08 | 5.8 |
| REGN15510 | 141.6 ± 1.3 | 30.9 | 2.95E+05 | 6.91E−04 | 2.34E−09 | 16.7 |

TABLE 13
Kinetic and Equilibrium Binding Parameters of Monomeric Cynomolgus 4-1BB
to Surface-captured Multispecific PSMA × 4-1BB 1 + 2 Format Antibodies

| Multispecific | mAb | h4-1BB.mmH | | | | |
|---|---|---|---|---|---|---|
| Antibody | Capture | Bound at | kₐ | k_{d} | K_{D} | t½ |
| Designation | (RU) | 270 nM (RU) | (1/Ms) | (1/s) | (M) | (min) |
| REGN15505 | 160.3 ± 4.0 | 32.2 | 1.14E+05 | 1.60E−03 | 1.41E−08 | 7.2 |
| REGN15510 | 140.2 ± 3.5 | 32.4 | 1.49E+05 | 1.77E−03 | 1.19E−08 | 6.5 |

TABLE 14
Lack of Monomeric Murine 4-1BB Binding
to Surface-captured Multispecific PSMA ×
4-1BB 1 + 2 Format Antibodies

| Multispecific | mAb | m4-1BB.mmH | | | | |
|---|---|---|---|---|---|---|
| Antibody | Capture | Bound at | kₐ | k_{d} | K_{D} | t½ |
| Designation | (RU) | 270 nM (RU) | (1/Ms) | (1/s) | (M) | (min) |
| REGN15505 | 168.9 | −8.4 | NB* | NB* | NB* | NB* |
| REGN15510 | 144.4 | −7.8 | NB* | NB* | NB* | NB* |

*NB = Non-binding

TABLE 15
Kinetic and Equilibrium Binding Parameters of Dimeric Human 4-1BB to Surface-
captured Multispecific PSMA × 4-1BB 1 + 2 Format Antibodies

| Multispecific | mAb | h4-1BB.mFc | | | | |
|---|---|---|---|---|---|---|
| Antibody | Capture | Bound at | kₐ | k_{d} | K_{D} | t½ |
| Designation | (RU) | 90 nM (RU) | (1/Ms) | (1/s) | (M) | (min) |
| REGN15505 | 161.3 ± 1.2 | 49.6 | 2.15E+05 | 6.34E−05 | 2.95E−10 | 182.2 |
| REGN15510 | 139.9 ± 1.4 | 56.9 | 3.58E+05 | 1.09E−04 | 3.05E−10 | 105.9 |

### Example 3: Strong Binding of Multispecific PSMAx4-1BB Antibody to Primary T Cells and PSMA+ Cancer Cell Lines

This example relates to in vitro studies demonstrating the ability of a multispecific PSMAx4-1BB antibody to bind to human and cynomolgus primary T cells and the PSMA+ cancer cell lines C4-2 and 22RV1 as measured by flow cytometry. C4-2 is a cell line, which expresses PSMA, with epithelial-like morphology that was isolated from a human prostate cancer LNCaP cell subcutaneous xenograft tumor of castrated mouse. 22RV1 is a human prostate carcinoma epithelial cell line, which expresses PSMA. Raji is a Burkitt's Lymphoma cell line that does not express PSMA or 4-1BB. The following antibodies were used in this study: multispecific PSMAx4-1BB (REGN15505), bivalent parental anti-PSMA, bivalent parental anti-4-1BB, multispecific 4-1BB control (one arm binding an unrelated antigen and the other arm comprising two domains, each binding 4-1BB), and isotype control.

Primary T cells, cells of the PSMA+ cancer cell lines C4-2 and 22RV1, and PSMA-control Raji cells were seeded at 1×10⁵ cells/well. Human T cells were pre-activated with CD3/CD28 activation beads, and cynomolgus T cells were pre-activated with phytohemagglutinin (PHA). All cells were incubated for 30 minutes at 4° C. with a serial dilution, ranging from 200 nM to 2.56 pM, of REGN15505 and related antibodies. Fc block was used for human and cynomolgus T cell incubations.

After incubation, all cells were washed twice with cold PBS containing 1% filtered FBS. A fluorophore-conjugated anti-human secondary antibody was added to the cells in Flow cytometry staining buffer (eBioscience) containing 1 mM EDTA. Cells were incubated for an additional 30 minutes. Live/dead dye was added to Human and Cynomolgus T cells incubations. Wells containing secondary only were used as a control.

Human or Cynomolgus T cells were washed and stained with a cocktail of anti-CD2, anti-CD16, anti-CD4, and anti-CD8 antibodies in Brilliant Stain Buffer for an extra 20 min incubation at 4° C. After wash, the T cells were re-suspended in Flow cytometry staining buffer (eBioscience) containing 1 mM EDTA, and analyzed by Flow cytometry on a BD FACS Celesta cell sorter. C4-2, 22RV1, and Raji cells were washed, re-suspended in Flow cytometry staining buffer containing 1 mM EDTA and analyzed by flow cytometry on a BD FACS Celesta cell sorter. T cells were gated in a Live/CD2+/CD4+/CD16− or Live/CD2+/CD8+/CD16− gate; C4-2, 22RV1, and Raji cells were gated using forward scatter (FSC) and side scatter (SCC). MFI values for fluorescent dye APC were calculated using FlowJo™ software. MFI values were plotted using GraphPad Prism™ software and EC₅₀ values for the antibodies were determined from a four-parameter or three-parameter logistic equation over a dose-response curve. Last point plotted at the lowest concentration represents the well containing a secondary antibody only.

Results: The multispecific PSMAx4-1BB antibody REGN15505 bound to the surface of activated human T cells, cynogolmous T cells, and cells of two PSMA+ cancer cell lines with low nanomolar EC₅₀ values. Table 16 sets forth the EC₅₀ values for REGN15505 and related antibodies binding to each cell type.

TABLE 16
EC50 values of REGN15505 and Related Antibodies
to T cells and cells of PSMA⁺ cancer cell lines

| | EC₅₀ [M] | | | | | | |
|---|---|---|---|---|---|---|---|
| | Human | Human | Cyno | Cyno | | | |
| | CD4+ T | CD8+ T | CD4+ T | CD8+ T | | | |
| Antibody | cells | cells | cells | cells | C4-2 | 22RV1 | Raji |
| PSMA × | 2.30E−09 | 2.20E−09 | 6.50E−10 | 3.56E−10 | 9.00E−09 | 6.50E−09 | ND |
| 4-1BB | | | | | | | |
| Parental | ND | ND | ND | ND | 2.20E−09 | 1.10E−09 | ND |
| PSMA | | | | | | | |
| Parental | 1.60E−09 | 1.40E−09 | 4.34E−10 | 2.49E−10 | ND | ND | ND |
| 4-1BB | | | | | | | |
| Multispecific | 1.70E−09 | 2.20E−09 | 5.80E−10 | 2.42E−10 | ND | ND | ND |
| 4-1BB | | | | | | | |
| control | | | | | | | |
| Isotype | ND | ND | ND | ND | ND | ND | ND |
| control | | | | | | | |

ND: Not Determined, because no dose-dependent response was observed

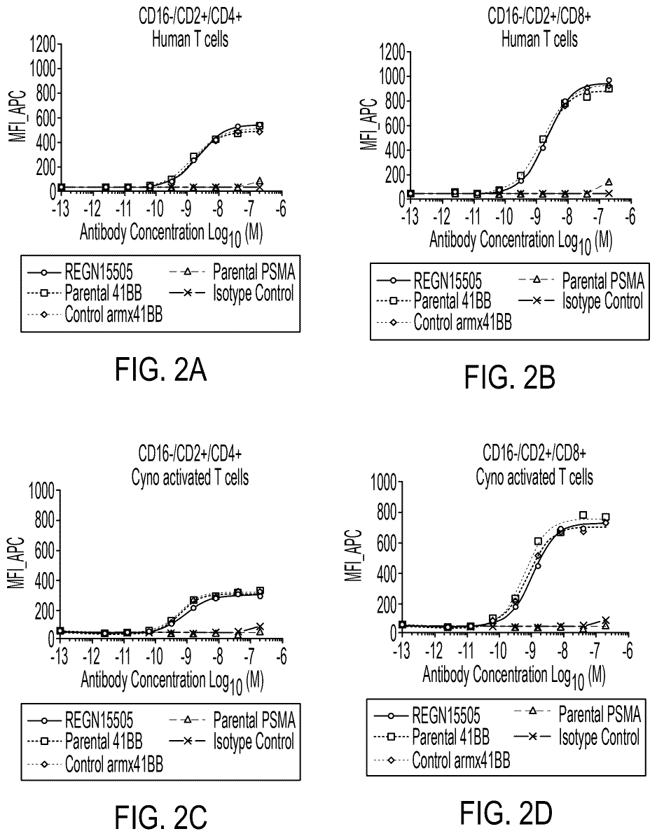

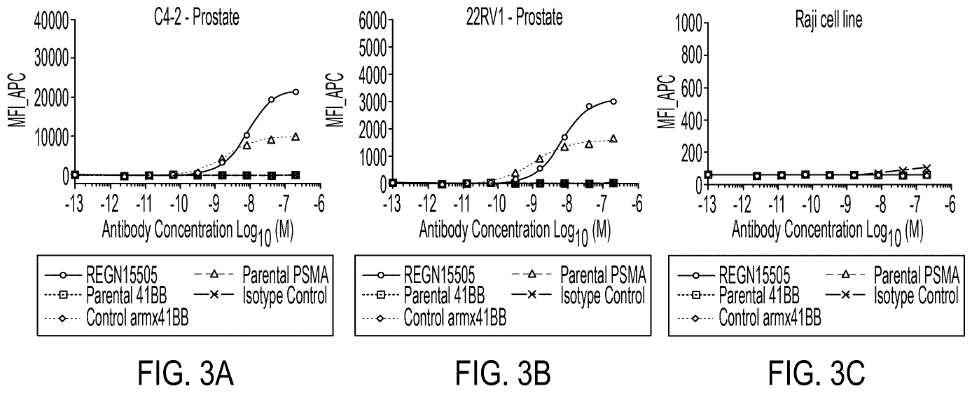

REGN15505 is capable of binding specifically to 4-1BB on T cells (FIGS. 2A-2D; Table 16) and to cancer cell lines expressing PSMA (FIGS. 3A-3B; Table 16). REGN15505 bound to both CD4+ and CD8+ activated human T cells, reaching saturation, comparable to the control parental 4-1BB antibody (FIGS. 2A-2B). REGN15505 bound CD4+ and CD8+ activated human T cells with EC₅₀ values of 2.3 E-09 M and 2.2E-09M, respectively (Table 16). REGN15505 bound to both CD4+ and CD8+ activated cynomolgus T cells, reaching saturation, comparable to the control parental 4-1BB antibody (FIGS. 2C-2D). REGN15505 bound CD4+ and CD8+ activated cynomolgus T cells with EC₅₀ values of 6.5E-10M and 3.6E-10M, respectively (Table 16). REGN15505 bound PSMA+ cells with lower max MFI compared to the bivalent parental PSMA antibody (FIGS. 3A-3B). REGN15505 bound to C4-2 cells with an EC₅₀ value of 9E-09M, and REGN15505 bound to 22RV1 cells with an EC₅₀ value of 6.5E-09M, and REGN15505 did not bind to RAJI cells (FIG. 3C; Table 16). The isotype control antibody did not exhibit any binding to human or cynomolgus T cells, nor did it bind to cell lines expressing PSMA (FIGS. 2A-2D and 3A-3C).

### Example 4: Robust Costimulatory Activity of a Multispecific PSMAx4-1BB Antibody

This example relates to an in vitro study demonstrating the ability of a multispecific PSMAx4-1BB antibody to enhance the cytotoxic potency of a multispecific CD3 antibody targeting an unrelated tumor-associated antigen (TAAxCD3). C4-2 is a TAA+; PSMA+ cell line with epithelial-like morphology that was isolated from a human prostate cancer LNCaP cell subcutaneous xenograft tumor of castrated mouse. The following antibodies were used in this study: multispecific PSMAx4-1BB (REGN15505), multispecific TAAxCD3, and multispecific 4-1BB control (one arm comprising two domains, each binding 4-1BB and the other arm binding an unrelated antigen).

In order to monitor the killing of cells expressing PSMA in the presence of a combination of a TAAxCD3 and the PSMAx4-1BB antibody REGN15505, C4-2 (TAA+; PSMA+) cells were labeled with 1 μM of the fluorescent tracking dye Violet Cell Tracker. After labeling, cells were plated overnight at 37° C. Separately, human peripheral blood mononuclear cells (PBMC) were plated in supplemented RPMI media at 1×10⁶ cells/mL and incubated overnight at 37° C. in order to enrich for lymphocytes by depleting adherent macrophages, dendritic cells, and some monocytes. The next day, target cells were co-incubated with adherent cell-depleted human PBMC (Effector/Target cell 4:1 ratio), a serial dilution of REGN15505 (concentration range: 200 nM to 50 pM) or multispecific 4-1BB control antibody and a fixed concentration of a TAAxCD3 (20 pM) for 5 days at 37° C.

Cells were removed from cell culture plates using Trypsin-EDTA dissociation buffer, and analyzed by flow cytometry on a FACS BD LSRFortessa-X20 cell sorter. For flow cytometry analysis, cells were stained with a dead/live Near IR Reactive dye (Invitrogen). 5×10⁵ counting beads were added to each well immediately before flow cytometry analysis. 1×10⁵ beads were collected for each sample. For the assessment of specificity of killing, cells were gated on live Violet labeled populations. Percent of live population was recorded and used for the calculation of survival. T cell activation was assessed by incubating cells with directly conjugated antibodies to CD2, CD4, CD8, CD25, PD1 and by reporting the percent of CD25+ or PD1+ T cells out of CD2+/CD4+ or CD2+/CD8+ T cells. The EC₅₀ values of the antibodies were determined from a four-parameter logistic equation over a dose-response curve using GraphPad Prism™ software.

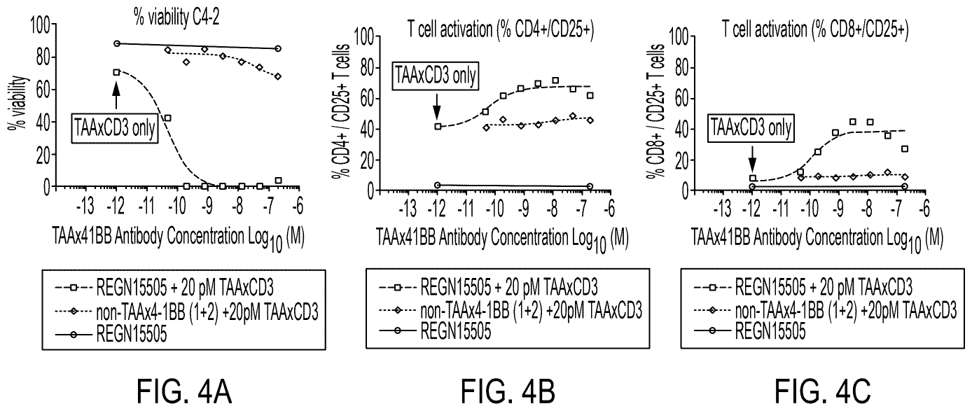

Results: The multispecific PSMAx4-1BB antibody REGN15505 successfully enhanced the cytotoxic potency of a fixed concentration (20 pM) of a TAAxCD3 multispecific antibody in the presence of human PBMC against C4-2 cells in a dose-dependent manner. (FIG. 4A). The multispecific 4-1BB control antibody did not show this effect (FIG. 4A). REGN15505 also enhanced the increase in T cell activation, with upregulation of CD25 expression on T cells (FIGS. 4B-4C). Table 17 sets forth the cytotoxicity and CD25 expression results of this study.

TABLE 17
Cytoxicity and CD25 expression

| | | | CD4+/CD25+ | CD8+/CD25+ |
|---|---|---|---|---|
| | | % | T cell | T cell |
| Signal 1 | Costimulatory | Cytotoxicity | Activation | Activation |
| antibody | antibody | EC₅₀ [M] | EC₅₀ [M] | EC₅₀ [M] |
| TAAxCD3 | PSMAx4-1BB | 4.50E−11 | 6.70E−11 | 1.40E−10 |
| | (REGN15505) | | | |
| | Multispecific | ND | ND | ND |
| | 4-1BB control | | | |
| None | PSMAx4-1BB | ND | ND | ND |
| | (REGN15505) | | | |

ND: Not Determined, because no dose dependent response was observed

### Example 5: Potent Ability of Multispecific PSMAx4-1BB Antibodies to Activate a T Cell Line Expressing 4-1BB

This example relates to an in vitro study demonstrating the ability of multispecific PSMAx4-1BB antibodies, in the presence of target cells expressing PSMA, to specifically stimulate 4-1BB signaling in a 4-1BB+ T cell line, as measured by increased expression of an NF-kB-inducible reporter gene.

Two signals, “signal 1” & “signal 2”, are required for proper T cell activation. “Signal 1” is induced by binding of the T cell receptor (TCR) on T cells to peptide-bound major histocompatibility complex (MHC) molecules on antigen presenting cells (APCs). “Signal 2” is provided by engagement of co-stimulatory receptors expressed on T cells, such as 4-1BB receptor, with ligands expressed on APC's, such as 4-1BBL. Therefore, activation of 4-1BB signaling provides a targeted approach to enhance existing TCR signaling.

Multispecific PSMAx4-1BB antibodies are designed to mimick the natural ligands of 4-1BB, by bridging PSMA+ target cells with 4-1BB+ T cells, to provide “signal 2” in order to enhance the activation of T cells in the presence of a “signal 1” provided, for example, by a tumor-associated antigen (TAA) x CD3 multispecific antibody.

The ability of antibodies targeting PSMA to interact with and activate 4-1BB, a costimulatory receptor found on T cells that acts as “signal 2” for proper T cell activation, was measured in an engineered reporter assay. In this assay, the reporter cells were Jurkat cells (a leukemia T cell line), engineered to express human 4-1BB and the reporter gene luciferase under the control of a NF-kB-inducible promoter (NFkB-Luc) (Jurkat/NFkB-Luc/h4-1BB cells). In this reporter assay, 4-1BB activation leads to NFkB promoter activity in the absence of signal 1. Three types of target cells were tested in this study: (i) HEK293 cells (a human embryonic kidney cell line) engineered to express CD20 and PSMA (HEK293/CD20/PSMA), (ii) control HEK293 cells expressing CD20 only (HEK293/CD20), and (iii) LNCaP cells (a human prostate adenocarcinoma line that expresses endogenous PSMA), engineered to express CD20 (LNCaP/CD20/endogPSMA). The following antibodies were used in this study: the multispecific PSMAx4-1BB (REGN15505 and REGN15510), bivalent 4-1BB, and two isotype controls.

Reporter cells were incubated with target cells and multispecific PSMAx41BB, or control, antibodies. The ability of PSMA targeted multispecifics to specifically bind PSMA+ target cells and subsequently engage and activate 4-1BB on Jurkat/NFkB-Luc/41BB reporter cells was evaluated via a luminescent readout.

One day before the experiment, Jurkat/NFkB-Luc/h4-1BB cells were split to 7.5×10⁵ cells/mL in growth media (RPMI+10% FBS+Penicillin/Streptomycin/L-glutamine (P/S/G)+1 μg/ml Puromycin). On the day of the experiment the cells were resuspended in assay media (RPMI+10% FBS+P/S/G) and were added to 384-well white plates at 1.5×10⁴ cells/well. Target cells (HEK293/CD20/PSMA, HEK293/CD20, and LNCaP/CD20/endogPSMA) were resuspended in assay media, then added to wells at 6.5×10³ cells/well. REGN15505, REGN15510, bivalent 4-1BB, and isotype control antibodies were prepared in assay media and titrated from 40 nM to 38 fM in a 1:4 dilution, the final point of the 12-point dilution containing no titrated antibody, and added to the appropriate wells. Plates were incubated at 37° C. and 5% CO₂ for 4.5 hours and then Promega ONE-Glo™ luciferase substrate was added to each well according to manufacturer's instructions. Luciferase activity was recorded as a luminescence signal using the ENVISION plate reader and expressed as relative light units (RLU).

EC₅₀ values were determined by a 4-parameter logistic equation over a 12-point response curve using GraphPad Prism™ software Signal recorded for the 12th point on the dilution curve (no antibody) was plotted at 9.5 fM. Maximal RLU is given as the mean max response detected within the tested dose range.

Results: When Jurkat/NFkB-Luc/4-1BB reporter cells were incubated with PSMA+target cells, the addition of the multispecific PSMAx4-1BB antibodies REGN15505 and REGN15510 resulted in a dose dependent increase in NF-kB activity (as measured by luciferase signal) (Tables 18 and 19). When Jurkat/NFkB-Luc/4-1BB reporter cells were incubated with HEK293/CD20/PSMA target cells, REGN15505 and REGN15510 resulted in a dose dependent increase in NF-kB activity, whereas the isotype control antibodies did not. When Jurkat/NFkB-Luc/4-1BB reporter cells were incubated with HEK293/CD20 target cells, which lack PSMA, no response was seen with REGN15505, REGN15510, or isotype controls. Importantly, when Jurkat/NFkB-Luc/4-1BB reporter cells were incubated with target cells that are a prostate cancer cell line that normally expresses PSMA (LNCaP/CD20/endogPSMA), REGN15505 and REGN15510 resulted in a dose dependent increase in NF-kB activity, whereas the isotype control antibodies did not.

Potency values and maximum luciferase activity of antibodies are set forth in Tables 18 and 19, respectively.

TABLE 18
Potency values for Multispecific PSMAx4-1BB and Control Antibodies

| | EC50 [M] | | | |
|---|---|---|---|---|
| | | | Jurkat/NFkB-Luc/ | Jurkat/NFkB-Luc/ |
| | Jurkat/NFkB- | Jurkat/NFkB-Luc/ | 4-1BB and | 4-1BB and |
| | Luc/4-1BB | 4-1BB and | HEK293/CD20/ | LNCaP/CD20/ |
| Antibody | alone | HEK293/CD20 | PSMA | endogPSMA |
| REGN15505 | ND | ND | 1.56E−10* | 2.33E−10* |
| REGN15510 | ND | ND | 4.17E−11** | 4.98E−11** |
| Bivalent | 1.90E−10 | 1.13E−10 | 1.26E−10 | 1.28E−10 |
| 4-1BB | | | | |
| Isotype | ND | ND | ND | ND |
| control 1 | | | | |
| Isotype | ND | ND | ND | ND |
| control 2 | | | | |

ND: Not Determined, because no dose dependent response was observed
*Values for the highest antibody concentration was removed due to a “hook effect”.
**Values for the highest 2 antibody concentrations were removed due to a “hook effect”.

TABLE 19
Maximum Reporter Activity for Multispecific
PSMAx4-1BB and Control Antibodies

| | Max Relative Light Units (RLU)* | | | |
|---|---|---|---|---|
| | | | Jurkat/NFkB-Luc/ | Jurkat/NFkB-Luc/ |
| | Jurkat/NFkB- | Jurkat/NFkB-Luc/ | 4-1BB and | 4-1BB and |
| | Luc/4-1BB | 4-1BB and | HEK293/CD20/ | LNCaP/CD20/ |
| Antibody | alone | HEK293/CD20 | PSMA | endogPSMA |
| REGN15505 | 2.34E+02 | 2.60E+02 | 9.99E+03 | 1.14E+04 |
| REGN15510 | 2.40E+02 | 2.38E+02 | 1.61E+04 | 1.46E+04 |
| Bivalent | 9.35E+03 | 6.06E+03 | 6.56E+03 | 7.76E+03 |
| 4-1BB | | | | |
| Isotype | 2.74E+02 | 2.58E+02 | 3.74E+02 | 2.38E+02 |
| control 1 | | | | |
| Isotype | 2.24E+02 | 2.86E+02 | 4.14E+02 | 2.14E+02 |
| control 2 | | | | |

ND: Not Determined, because no dose dependent response was observed
*Highest mean RLU value within tested dose-range

### Example 6: Potent Ability of Multispecific PSMAx4-1BB Antibodies to Activate Primary T Cells and Enhancement by PD-1 Blockade

This example relates to an in vitro study demonstrating the ability of multispecific PSMAx4-1BB antibodies to activate human primary T cells, as determined by IL2 and IFNγ release, by engaging PSMA and 4-1BB to deliver “signal 2”, in the presence of a PSMA+ human prostate cancer line, which provides an allogeneic TCR response serving as “signal 1”. The study also demonstrates the enhancement of primary T cell activation induced by an antagonist anti-programmed cell death protein 1 (anti-PD1) antibody in combination with multispecific PSMAx4-1BB antibody treatment.

Two signals, “signal 1” & “signal 2”, are required for proper T cell activation. “Signal 1” is induced by binding of the T cell receptor (TCR) on T cells to peptide-bound major histocompatibility complex (MHC) molecules on antigen presenting cells (APCs); whereas “signal 2” is provided by engaging co-stimulatory receptors on T cells. One such costimulatory receptor is 4-1BB. Expression of 4-1BB is induced on the surface of T cells after antigen- or mitogen-induced activation. Stimulation of 4-1BB occurs via engagement with the 4-1BB ligand 4-1BBL, present on APCs. Therefore, activation of 4-1BB signaling provides a targeted approach to enhance existing TCR signaling.

Multispecific PSMAx4-1BB antibodies are designed to mimick the natural ligand of 4-1BB, by bridging PSMA+ target cells with T cells expressing 4-1BB. Therefore, in the presence of PSMA+ target cells, multispecific PSMAx4-1BB antibodies would provide “signal 2” in order to enhance the activation of T cells in the presence of a “signal 1”, provided by an allogeneic response provided by the APC. However, T cell activation can be inhibited by the ligation of the receptor PD-1 on T cells to its ligand PD-L1 on APCs. Ligated PD-1 leads to the recruitment of phosphatases to CD28 and the TCR complex (Zou and Chen. Nat. Rev. (2008) 8:467-477; Hui et al. Science (2017) 355 (6332): 1428-33), which in turn counteract TCR signaling and 4-1BB stimulation. Thus, blockade of the PD-1/PD-L1 interaction in combination with multispecific PSMAx4-1BB antibodies may potentiate T cell function and promote killing of target cells such as in cancer.

Release of IL2 and IFNγ by human primary T cells was measured in response to treatment with multispecific PSMAx4-1BB and control antibodies in the presence of a human prostate cancer line engineered to express human PSMA. The effect on IL2 and IFNγ release upon addition of the antagonistic anti-PD-1 antibody cemiplimab was also assessed.

The following antibodies were used in this study: multispecific PSMAx4-1BB antibodies (REGN15505 and REGN15510), non-TAAx4-1BB, anti-PD-1 (cemiplimab), and cemiplimab-matched isotype control. DU-145/hPSMA cells and human peripheral blood mononuclear cells (PBMCs) were used in this study. DU-145/hPSMA is a human prostate carcinoma cell line, derived from a central nervous system metastasis, that was genetically engineered to stably overexpress human PSMA. Human PBMCs were isolated from a healthy donor leukocyte pack from Precision for Medicine using the EasySep™ Direct Human PBMC Isolation Kit, following the manufacturers protocol, and frozen down. CD3+ T cells were isolated from thawed PBMCs using an EasySep™ Human CD3+ T Cell Isolation Kit from StemCell Technologies, following the manufacturer's instructions.

IL2 and IFNγ Release Assay: Enriched CD3+ T cells, resuspended in stimulation media, were seeded in 96-well round bottom plates at 1×10⁵ cells/well. DU-145/hPSMA cells were added to CD3+ T cells at 5×10⁴ cells/well. Test antibodies were titrated from 380 fM to 25 nM in a 1:4 dilution and added to wells. The final point of the 10-point dilution contained no titrated antibody. Following addition of titrated antibody, a constant 10 nM of either cemiplimab or its matched isotype control was added. Plates were incubated for 96 hours at 37° C., 5% CO₂ and 20 μL of supernatant was removed and used for measuring IL2 as well as 20 μL of supernatant for measuring IFNγ. The amount of cytokine in assay supernatant was determined using AlphaLisa kits from PerkinElmer following the manufacturer's protocol with modifications recommended by the manufacturer for enhanced cytokine detection. These modifications included adjusting the supernatant volume for testing from 5 μL to 20 μL, while maintaining the recommended bead and antibody concentration, and increasing the incubation time with the anti-analyte acceptor bead/biotinylated antibody mix from one hour to two hours. Cytokine measurements were acquired on Perkin Elmer's multilabel plate reader Envision, and values were reported as pg/mL. EC₅₀ values were determined from a four-parameter logistic equation over a 10-point dose-response curve using GraphPad Prism™ software. Maximal IL2 and IFNγ is given as the mean max response detected within the tested dose range.

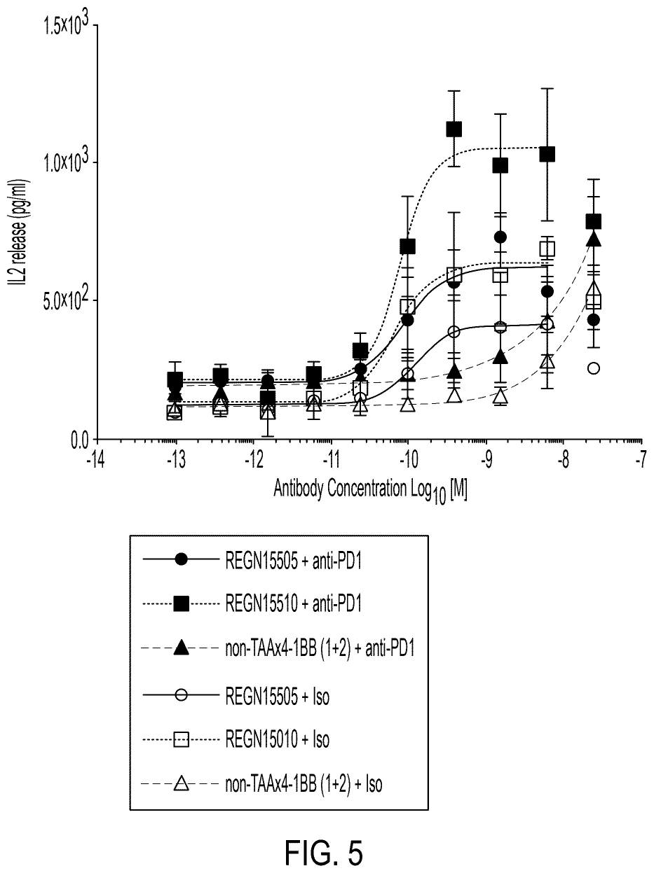

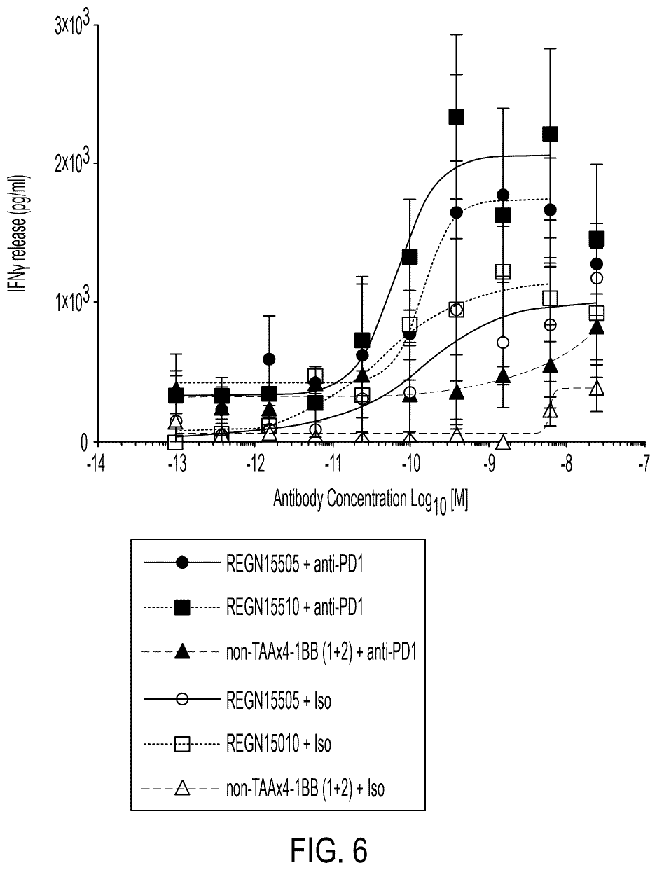

Results: In the presence of an allogeneic prostate cancer cell line expressing PSMA, DU-145/hPSMA, REGN15505 and REGN15510 treatment of human primary T cells resulted in dose dependent increases in IL2 and IFNγ release (Table 20; FIGS. 5, 6). Addition of cemiplimab enhanced this effect. The maximum cytokine release in response to combination treatment of REGN15505 or REGN15510 and cemiplimab was greater compared to treatment with REGN15505 or REGN15510 and cemiplimab-matched isotype control (Table 20; FIGS. 5, 6).

In the presence of DU-145/hPSMA target cells and allogeneic stimulation, REGN15505 and REGN15510 induced a dose dependent increase in IL2 release (Table 20; FIG. 5). REGN15510 induced the highest maximum cytokine release among all antibodies tested. Cemiplimab increased maximum cytokine release in comparison to cemiplimab-matched isotype control. In the presence of DU-145/hPSMA target cells and allogeneic stimulation, the REGN15505, and REGN15510 induced a dose dependent increase in IFNγ release Table 20; FIG. 6). REGN15510 led to the highest maximum cytokine release among all antibodies tested. Cemiplimab increased maximum cytokine release in comparison to cemiplimab-matched isotype control.

Table 20 sets forth the maximum IL2 and IFNγ release and potency values of antibodies.

TABLE 20
Maximum IL2 and IFNγ Release and Potency Values of Antibodies

| | DU-145/hPSMA | | | | | | | |
|---|---|---|---|---|---|---|---|---|
| | IL2 | | | | IFNγ | | | |
| | +isotype control | | +cemiplimab | | +isotype control | | +cemiplimab | |
| | MAX | EC₅₀ | MAX | EC₅₀ | MAX | EC₅₀ | MA | EC₅₀ |
| Antibodies | [pg/ml] | [M] | [pg/ml] | [M] | [pg/ml] | [M] | [pg/ml] | [M] |
| REGN15505 | 416.8 | 1.23E−10* | 729.4 | 8.74E−11* | 1180.7 | 1.49E−10 | 1774.8 | 1.47E−10* |
| REGN15510 | 690.8 | 6.54E−11* | 1124.3 | 7.98E−11* | 1224.0 | 4.07E−11* | 2335.0 | 6.70E−11* |
| Non-TAA × | 546.8 | NC | 737.0 | NC | 391.5 | NC | 831.3 | NC |
| 4-1BB | | | | | | | | |

NC: Not Calculated, because the data did not fit a 4-parameter logistic equation.
Maximum [pg/ml] is the highest mean pg/ml value within tested dose-range.
*While all data points are used to determine Max [pg/ml] of cytokine released, for EC₅₀ calculation the values for the highest antibody concentrations were removed due to a “hook effect”

### Example 7: Robust Anti-Tumor Efficacy with Advantageous Cytokine-Release Profile of a Multispecific PSMAx4-1BB Antibody in the Syngenic MC38/hPSMA Tumor Model

This example relates to an in vivo study demonstrating the efficacy of a multispecific PSMAx4-1BB antibody in treating human PSMA-expressing tumors in a murine model of colon cancer in comparison to treatment with 4-1BB control antibodies either alone or in combination with anti-PD-1. The study also demonstrates the beneficially low levels of cytokine release with administration of a multispecific PSMAx4-1BB antibody in comparison to administration of the anti-4-1BB control antibodies either alone or in combination with anti-PD-1. Mice were humanized for PSMA, CD3, CD28, and 4-1BB, wherein the mouse genes were replaced with their human homologues (hPSMA/hCD3/hCD28/h4-1BB mice), and tumors were generated from MC38 colon carcinoma cells, wherein the mouse PSMA gene was replaced with human PSMA (M38/hPSMA tumor cells). The following antibodies were used in this study: multispecific PSMAx4-1BB (REGN15505); multispecific-matched isotype control; multispecific anti-4-1BB control; bivalent anti-4-1BB; anti-mPD-1 (BE0146 (Bio X Cell)); and anti-PD-1-matched isotype control.

On Day 0, 8-12 week old hPSMA/hCD3/hCD28/h4-1BB mice (n=5-6/group) were implanted with 0.5×10⁶ M38/hPSMA tumor cells subcutaneously on the right flank. Antibodies were administered intraperitoneally (IP) on Days 7, 10, 14, 17, and 21 at a dose of 5 mg/kg with the exception of REGN15505, which was administered at 0.05, 0.5, or 5 mg/kg. Concentration of cytokines in serum from mice were measured 4 hours postdosing on Day 14. Tumors were measured approximately twice per week using calipers. Tumor volume was calculated using the formula X*Y*(X/2), where Y is the longest dimension and X is the perpendicular dimension, and mice with tumors larger than 2000 mm³ or with ulcerated tumors were euthanized by CO₂ asphyxiation. Survival was recorded until end of experiment at Day 60.

Results: Table 21 sets forth the average tumor volume at each time point post implantation of M38/hPSMA tumors treated with the indicated antibodies without anti-PD-1.

TABLE 21
Average Tumor Volume for M38/hPSMA Tumors Treated
with Multi-specific Antibodies Without Anti-PD-1

| | Antibody Treatment - includes isotype control for anti-PD-1 | | | | |
|---|---|---|---|---|---|
| | Multispecific | Multispecific | | | |
| | isotype | 4-1BB | 0.05 mg/kg | 0.5 mg/kg | 5 mg/kg |
| Days Post | control | control | REGN15505 | REGN15505 | REGN15505 |
| Implantation | Tumor Volume (mm³) | | | | |
| 7 | 27 | 23 | 24 | 24 | 23 |
| 10 | 52 | 60 | 46 | 36 | 37 |
| 14 | 143 | 123 | 112 | 127 | 41 |
| 17 | 348 | 379 | 399 | 313 | 46 |
| 21 | 816 | 983 | 988 | 696 | 60 |

Table 22 sets forth the average tumor volume at each time point post implantation of M38/hPSMA tumors treated with the indicated antibodies in combination with anti-PD-1.

TABLE 22
Average Tumor Volume for M38/hPSMA Tumors Treated with
Multi-specific Antibodies in Combination with Anti-PD-1

| | Antibody Treatment - includes anti-PD-1 | | | | | |
|---|---|---|---|---|---|---|
| | Multispecific | Multispecific | | | | |
| | isotype | 4-1BB | 0.05 mg/kg | 0.5 mg/kg | 5 mg/kg | Bivalent |
| Days Post | control | control | REGN15505 | REGN15505 | REGN15505 | 4-1BB |
| Implantation | Tumor Volume (mm³) | | | | | |
| 7 | 23 | 23 | 24 | 24 | 25 | 26 |
| 10 | 39 | 40 | 50 | 37 | 50 | 37 |
| 14 | 84 | 83 | 125 | 53 | 51 | 53 |
| 17 | 219 | 201 | 288 | 197 | 78 | 157 |
| 21 | 465 | 415 | 532 | 378 | 209 | 264 |

Table 23 sets forth survival at each time point post implantation of M38/hPSMA tumor cells of mice administered the indicated antibodies without anti-PD-1.

TABLE 23
Survival of Mice Implanted with M38/hPSMA Tumor Cells
and Administered Specific Antibodies Without Anti-PD-1

| | Antibody Treatment - includes isotype control for anti-PD-1 | | | | |
|---|---|---|---|---|---|
| | Multispecific | Multispecific | | | |
| | isotype | 4-1BB | 0.05 mg/kg | 0.5 mg/kg | 5 mg/kg |
| Days Post | control | control | REGN15505 | REGN15505 | REGN15505 |
| Implantation | Number of Mice Surviving at Each Time Point | | | | |
| 7 | 6 | 6 | 6 | 6 | 6 |
| 10 | 6 | 6 | 6 | 6 | 6 |
| 14 | 6 | 6 | 6 | 6 | 6 |
| 17 | 6 | 6 | 6 | 6 | 6 |
| 21 | 5 | 5 | 5 | 4 | 6 |
| 24 | 3 | 2 | 2 | 3 | 6 |
| 28 | 1 | 1 | 1 | 3 | 6 |
| 31 | 0 | 1 | 1 | 3 | 5 |
| 34 | | 0 | 1 | 2 | 2 |
| 38 | | | 0 | 1 | 2 |
| 42 | | | | 1 | 2 |
| 45 | | | | 1 | 2 |
| 60 | | | | 1 | 2 |

Table 24 sets forth survival at each time point post implantation of M38/hPSMA tumor cells of mice administered the indicated antibodies in combination with anti-PD-1.

TABLE 24
Survival of Mice Implanted with M38/hPSMA Tumor Cells and Administered
Specific Antibodies In Combination With Anti-PD-1

| | Antibody Treatment - includes anti-PD-1 | | | | | |
|---|---|---|---|---|---|---|
| | Multispecific | Multispecific | | | | |
| | isotype | 4-1BB | 0.05 mg/kg | 0.5 mg/kg | 5 mg/kg | Bivalent |
| Days Post | control | control | REGN15505 | REGN15505 | REGN15505 | 4-1BB |
| Implantation | Tumor Volume (mm³) | | | | | |
| 7 | 6 | 6 | 6 | 6 | 6 | 4 |
| 10 | 6 | 6 | 6 | 6 | 6 | 4 |
| 14 | 6 | 6 | 6 | 6 | 6 | 4 |
| 17 | 6 | 6 | 6 | 6 | 6 | 4 |
| 21 | 5 | 6 | 6 | 6 | 6 | 4 |
| 24 | 4 | 5 | 3 | 5 | 5 | 4 |
| 28 | 4 | 4 | 3 | 5 | 5 | 3 |
| 31 | 3 | 3 | 3 | 2 | 5 | 3 |
| 34 | 3 | 2 | 3 | 2 | 5 | 3 |
| 38 | 2 | 2 | 3 | 2 | 4 | 2 |
| 42 | 1 | 2 | 3 | 2 | 4 | 1 |
| 45 | 1 | 2 | 2 | 2 | 4 | 1 |
| 60 | 1 | 2 | 2 | 2 | 4 | 1 |

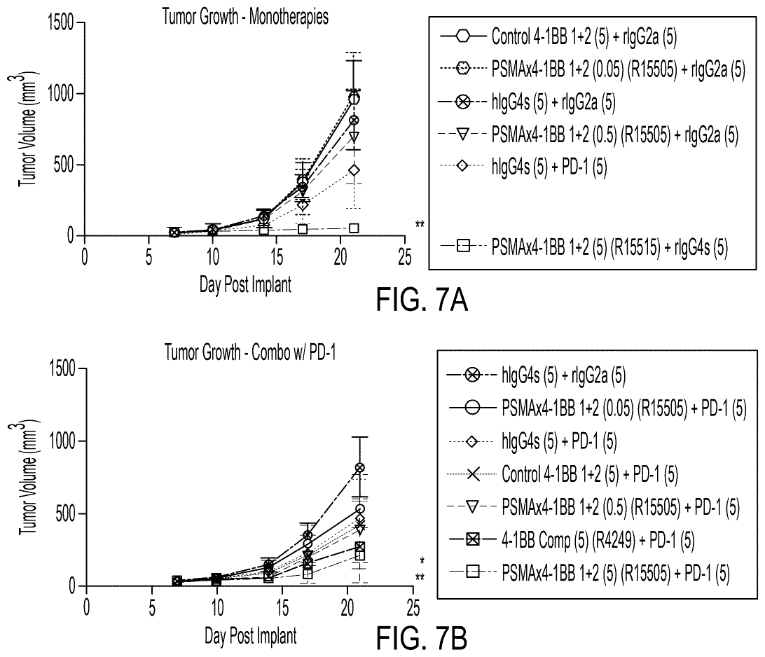

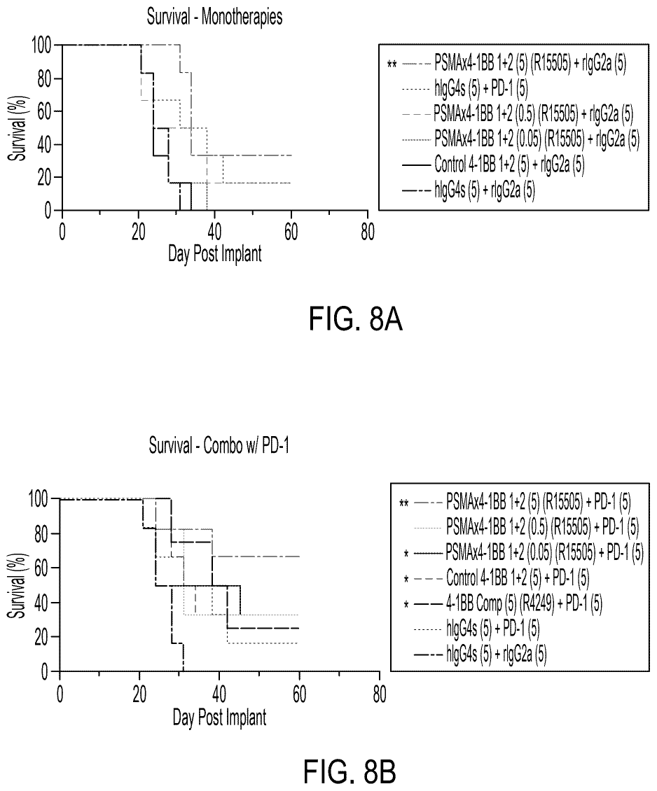

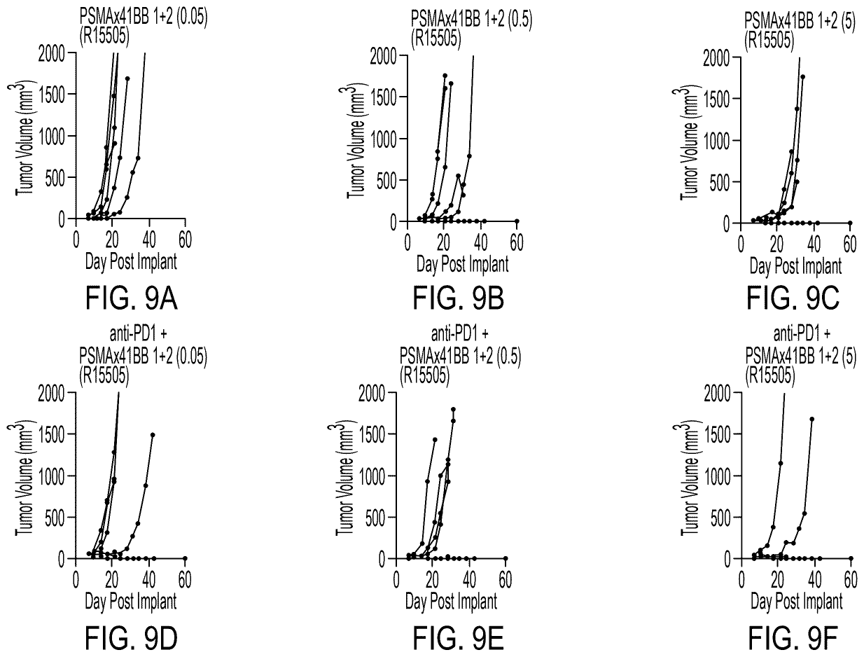

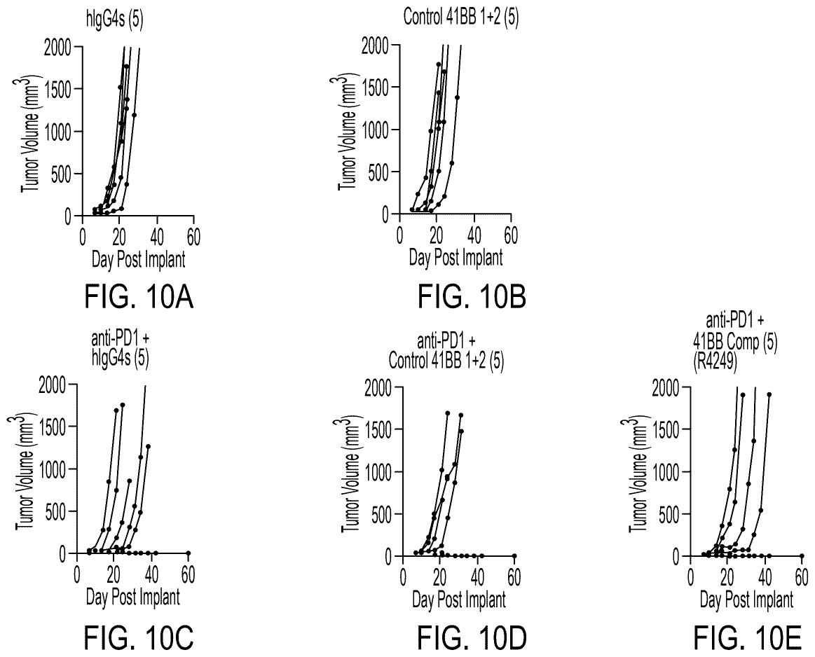

Administration of the multispecific PSMAx4-1BB antibody REGN15505 as a monotherapy to mice bearing MC38/hPSMA tumors potently controlled tumor growth and supported survival in a dose-dependent fashion (Tables 21 and 23; FIGS. 7A, 8A, 9A-9C). The ability of REGN15505 to control tumor growth and support survival was significantly greater than the ability of anti-PD-1, multispecific 4-1BB control, or multispecific isotype control to control tumor growth and support survival (Tables 21-23; FIGS. 7A-7B, 8A-8B, 9C, and 10A-10C). Combination therapy of REGN15505 and anti-PD-1 resulted in greater tumor control and inducement of survival than either antibody as monotherapy (Tables 21-24, FIGS. 7A-7B, 8A-8B, 9C-9F, and 10A-10E). Finally, while combination therapy of REGN15505 and anti-PD-1 was significantly more effective at controlling tumor growth and supporting survival than combination therapy of bivalent 4-1BB antibody and anti-PD-1, administration of REGN15505 resulted in a dramatically better cytokine-release profile than administration of bivalent 4-1BB antibody (Tables 22 and 24; FIGS. 8A-8B, 9F, 10E, and 11A-11J).

REGN15505 controlled tumor growth and supported survival in a dose-dependent fashion. For example, on Day 21 post implantation with MC38/hPSMA tumor cells, the average tumor volume of mice administered REGN15505 at a dose of 0.05 mg/kg (988 mm³) was comparable to multispecific isotype control (816 mm³) (Table 21; FIG. 7A). However, control of tumor growth by REGN15505 improved to an average tumor volume of 696 mm³ at a dose of 0.5 mg/kg and further improved to an average tumor volume of only 60 mm³ at a dose of 5 mg/kg (Table 21; FIG. 7A). This is a 13.6 fold improvement of 5 mg/kg REGN15505 over isotype control. Regarding survival, no mice out of 6 administered isotype control survived past Day 28, for example, and no mice out of 6 administered REGN15505 at a dose of 0.05 mg/kg survived past Day 31 (Table 23; FIG. 8A). However, survival of mice induced by REGN15505 improved to 1 mouse surviving to end of experiment at Day 60 at a dose of 0.5 mg/kg and further improved to 2 mice surviving at Day 60 at a dose of 8 mg/kg (Table 23; FIG. 8A).

The ability of REGN15505 to control tumor growth and support survival was significantly greater than the ability of anti-PD-1, multispecific 4-1BB control, or multispecific isotype control to control tumor growth and support survival (Table 21, antibody columns 1, 2 and 5; Table 22, antibody column 1; Table 23, antibody columns 1, 2, and 5; Table 24, antibody column 1; FIGS. 7A-7B, 8A-8B, 9C, and 10A-10C). For example, at Day 21 post implantation with MC38/hPSMA tumor cells, the average tumor volume of mice administered REGN15505 at 5 mg/kg was only 60 mm³ (Table 21, antibody column 5). In contrast, also at Day 21, the average tumor volume of mice administered anti-PD-1 at a dose of 5 mg/kg was 465 mm³ (Table 22, antibody column 1). Thus, at Day 21 tumors treated with anti-PD-1 were 7.75x larger than tumors treated with REGN15505. At Day 21, average tumor size of mice administered multispecific 4-1 BB control was 983 mm³ (i.e., 16.4x larger than tumors treated with REGN15505) (Table 21, antibody column 2); and average tumor size of mice administered multispecific isotype control was 816 mm³ (Table 21, antibody column 1). Regarding survival, at Day 31, for example, 5 out of 6 mice administered REGN15505 at a dose of 5 mg/kg survived (Table 23, antibody column 5). In contrast, also at Day 31, only 3 out of 6 mice administered anti-PD-1 at a dose of 5 mg/kg survived (Table 24, antibody column 1), and only 1 out of 6 mice administered multispecific 4-1BB control survived. No mice out of 6 administered multispecific isotype control survived at this time point (Table 23, antibody column 1).

Combination therapy of REGN15505 and anti-PD-1 resulted in greater tumor control and support of survival than either antibody as monotherapy (Tables 21-24, FIGS. 7A-7B, 8A-8B, 9C-9F, and 10A-10E). For example, at Day 21 post implantation with MC38/hPSMA tumor cells, average tumor volume of mice administered a combination therapy of anti-PD-1 at a dose of 5 mg/kg and REGN15505 at a dose of 0.5 mg/kg was 378 mm³ (Table 22, antibody row 4). In contrast, 5 mg/kg anti-PD-1 as monotherapy was less effective at inhibiting tumor growth, resulting in an average tumor volume of 465 mm³ at Day 21 (Table 22, antibody row 1), and 0.5 mg/kg REGN15505 as monotherapy was also less effective, resulting in an average tumor volume of 696 mm³ (Table 21, antibody row 4). Regarding survival, at end of experiment at Day 60, for example, 4 out of 6 mice administered combination therapy of 5 mg/kg anti-PD-1 and 5 mg/kg REGN15505 survived (Table 24, antibody column 5). In contrast, only 1 out of 6 mice administered 5 mg/kg anti-PD-1 as monotherapy survived (Table 24, antibody column 1), and only 2 out of 6 mice administered 5 mg/kg REGN15505 survived (Table 23, antibody column 5).

Finally, while combination therapy of REGN15505 and anti-PD-1 was significantly more effective at controlling tumor growth and supporting survival than combination therapy of bivalent 4-1BB antibody and anti-PD-1, administration of REGN15505 resulted in a dramatically better cytokine-release profile than administration of bivalent 4-1BB antibody (Tables 22 and 24;

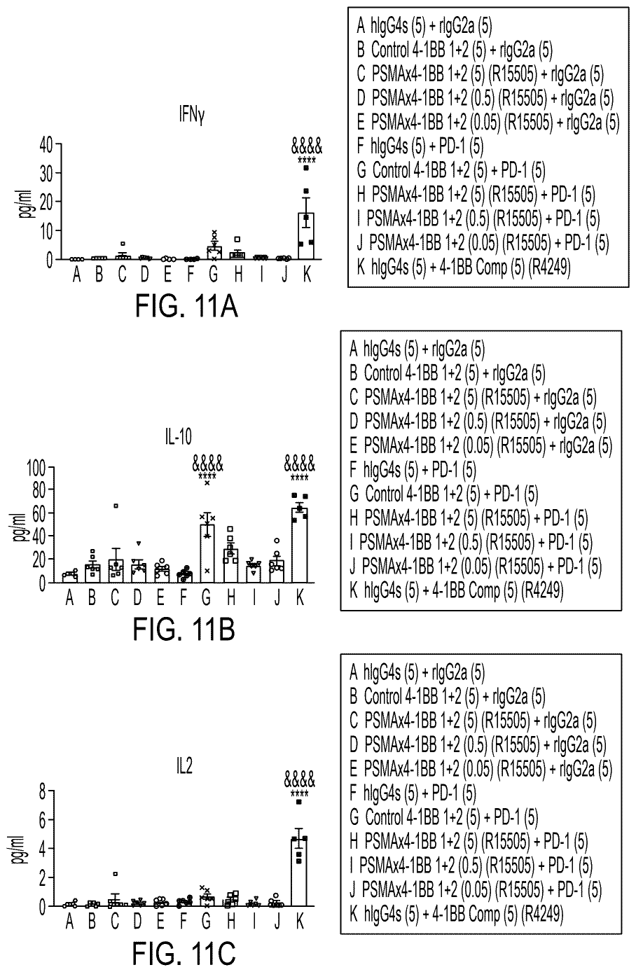

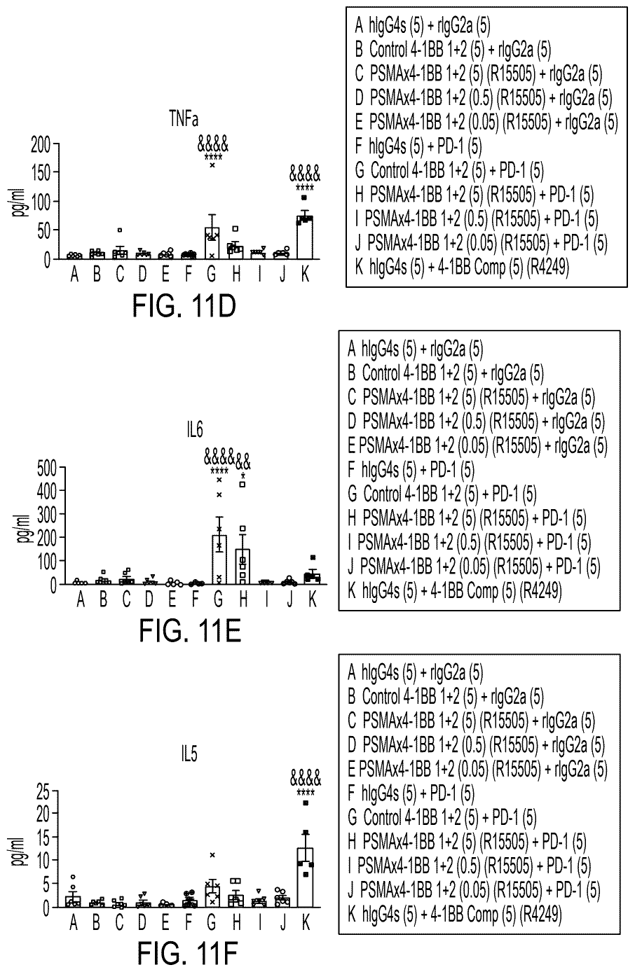

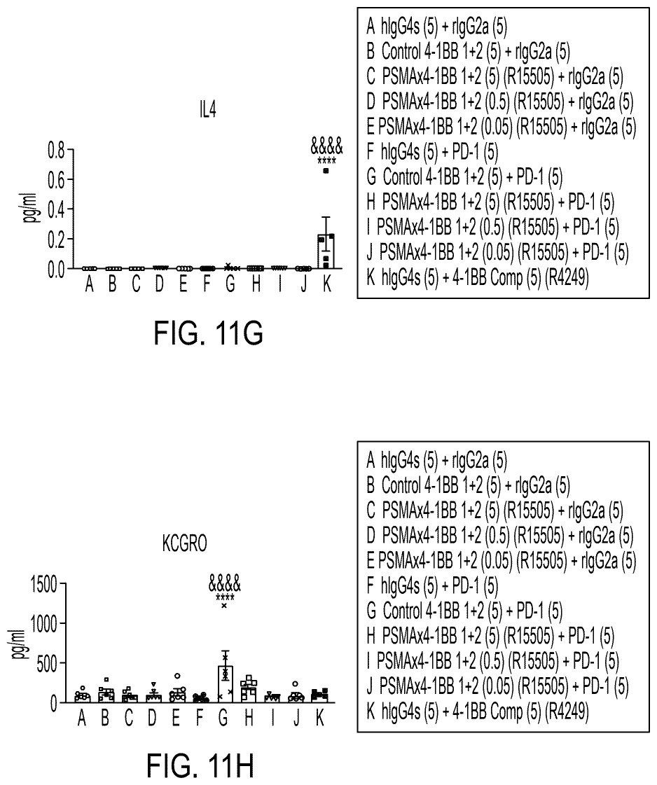

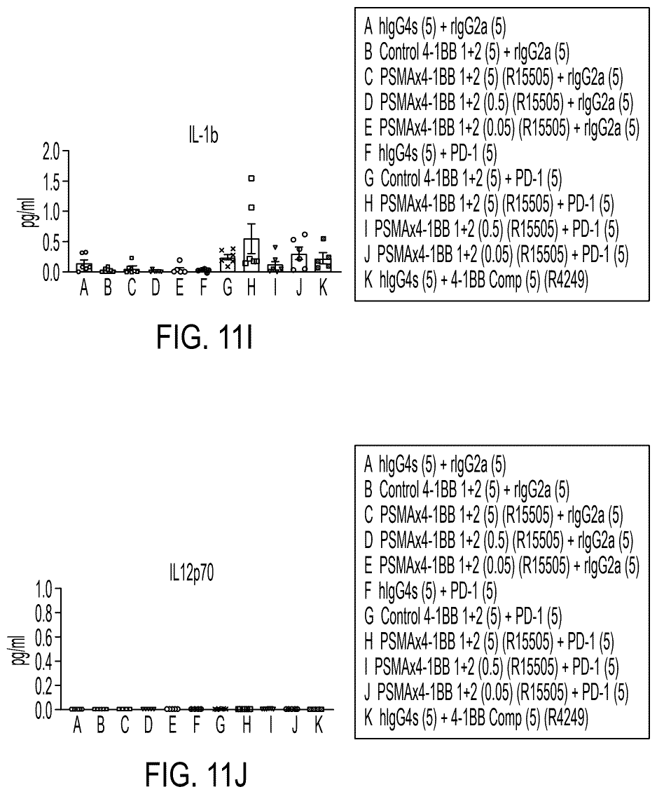

FIGS. 8A-8B, 9F, 10E, and 11A-11J). For example, at Day 21 post implantation with MC38/hPSMA tumor cells, mice administered the combination therapy of REGN15505 and anti-PD-1 showed an average tumor volume of 209 mm³ (Table 22). In contrast, mice administered the combination therapy of bivalent 4-1BB antibody and anti-PD-1 showed an average tumor volume of 264 mm³ (Table 22). Regarding survival, mice administered the combination therapy of REGN15505 and anti-PD-1 showed a dramatically higher survival rate than mice administered the combination therapy of bivalent 4-1BB antibody and anti-PD-1 (Table 24). For example, while 4 out of 6 (i.e., 67%) mice administered REGN15505 and anti-PD-1 survived until at end of experiment at Day 60, only 1 out of 4 (i.e., 25%) mice administered bivalent 4-1BB antibody and anti-PD-1 survived until Day 60. Excessive cytokine release (or “cytokine storm” is a dangerous phenomenon in cancer treatment. Surprisingly, despite the greater anti-cancer efficacy of REGN15505 compared to the efficacy of bivalent anti-4-1BB antibody, the cytokine release induced by REGN15505 was significantly lower than the amount of cytokine release induced by bivalent anti-4-1BB antibody (FIGS. 11A-11J). This effect was particularly pronounced for the cytokines IFNγ, IL-10, IL2, TNFα, IL5, and IL4 (FIGS. 11A-11D and 11F-11G).

### Example 8: Long-lived Anti-tumor Immunity Induced by Multispecific PSMAx4-1BB Antibody in the Syngenic MC38/hPSMA Tumor Model

This example relates to an in vivo study demonstrating the ability of combination therapy of a multispecific PSMAx4-1BB antibody and anti-PD-1 antibody to induce long-lived, CD8 dependent anti-tumor immunity to secondary tumor challenge in mice implanted with MC38/hPSMA tumor cells. Mice were humanized for PSMA, CD3, CD28, and 4-1BB, wherein the mouse genes were replaced with their human homologues (hPSMA/hCD3/hCD28/h4-1BB mice), and tumors were generated from MC38 colon carcinoma cells, wherein the mouse PSMA gene was replaced with human PSMA (M38/hPSMA tumor cells). The following antibodies were used in this study, each at a dose of 5 mg/kg: multispecific PSMAx4-1BB (REGN15505), anti-mPD-1 (BE0146 (Bio X Cell)); and isotype control.

On Day 0, hPSMA/hCD3/hCD28/h4-1BB mice were implanted with 0.5×10⁶ M38/hPSMA tumor cells subcutaneously on the right flank. The combination of REGN15505 and anti-PD-1 was administered intraperitoneally (IP) on Days 7, 10, 14, 17, and 21. An unimplanted group of hPSMA/hCD3/hCD28/h4-1BB mice were administered isotype control. Tumor-free mice were identified at Day 72, when 0.5×10⁶ M38/hPSMA tumor cells were implanted subcutaneously on the left flank of tumor-free and control mice. One group of the tumor-free mice were administered a CD8 depleting antibody (BE0061 (BioXcell)) at 300 μg per mouse one day prior to secondary implant and twice per week thereafter. Tumors were measured approximately twice per week using calipers until end of experiment at Day 25. Tumor volume was calculated using the formula X*Y*(X/2), where Y is the longest dimension and X is the perpendicular dimension, and mice with tumors larger than 2000 mm³ or with ulcerated tumors were euthanized by CO₂ asphyxiation. Naïve hPSMA/hCD3/hCD28/h4-1BB mice implanted with 0.5×10⁶ M38/hPSMA tumor cells subcutaneously on the left flank were used as controls.

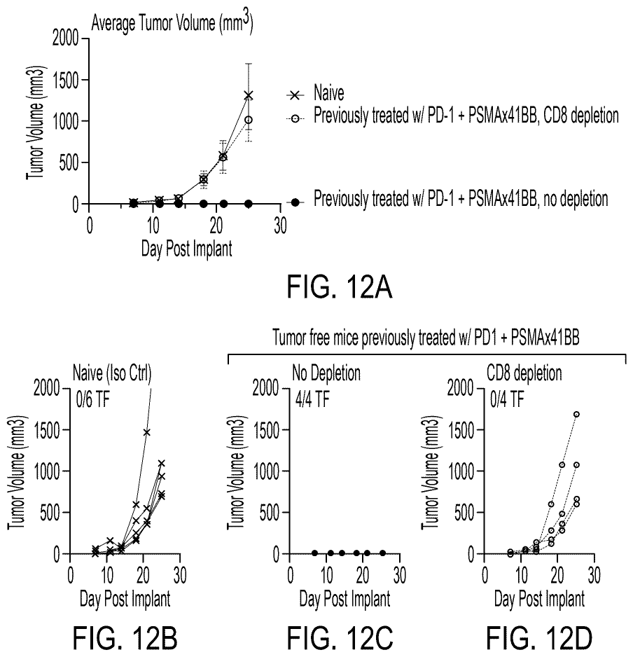

Results: Administration of combination therapy of REGN15505 and anti-PD-1 induces long-lived, CD8 dependent anti-tumor immunity to secondary tumor challenge. While control mice displayed normal turmor growth, the mice that had been previously implanted and had showed complete regression of the first tumor in response to combination therapy of REGN15505 and anti-PD-1 showed no growth of second tumors (FIGS. 12A-12D). While 0 out of 6 control mice were tumor-free, 4 out of 4 mice previously treated with combination therapy were tumor-free (FIGS. 12A-12D). Rejection of the second tumor implant without additional treatment indicated that these mice were immune to secondary tumor challenge. This was a striking and surprising result. CD8 depletion resulted in tumor growth similar to control mice (FIGS. 12A-12D), indicating that anti-tumor immunity was CD8 dependent.

### Example 9: Potent Tumor Growth Control and Survival with Multispecific PSMAx4-1BB Antibody in Combination with PSMAxCD3 Antibody in a Syngenic Trampc2/hPSMA Tumor Model without Excessive Cytokine Release

This example relates to an in vivo study demonstrating the ability of a multispecific PSMAx4-1BB antibody to synergistically enhance the inhibition of tumor growth and improvement in survival by PSMAxCD3 antibody administration to mice bearing syngenic Trampc2/hPSMA tumors. The study also demonstrates the beneficially low levels of cytokine release with administration of a combination therapy of multispecific PSMAx4-1BB antibody and PSMAxCD3 antibody in comparison to PSMAxCD3 antibody monotherapy and combination therapy of bivalent 4-1BB antibody and PSMAxCD3 antibody.

Mice were humanized for PSMA, CD3, CD28, and 4-1BB, wherein the mouse genes were replaced with their human homologues (hPSMA/hCD3/hCD28/h4-1BB mice), and tumors were generated from Trampc2 cells, wherein the mouse PSMA gene was replaced with human PSMA (Trampc2/hPSMA tumor cells). The following antibodies were used in this study: multispecific PSMAx4-1BB (REGN15505) at a dose of 5 mg/kg; multispecific-matched isotype control at a dose of 5 mg/kg; bivalent anti-4-1BB at a dose of 5 mg/kg; and PSMAxCD3 antibody at a dose of 0.02 mg/kg (“low dose”).

On Day 0, hPSMA/hCD3/hCD28/h4-1BB mice (n=7-8/group) were implanted with 5×10⁶ Trampc2/hPSMA tumor cells subcutaneously on the right flank. Antibodies were administered intraperitoneally (IP) on Days 0, 3, and 7. Concentration of cytokines in serum from mice were measured 4 hours postdosing on Day 0 and Day 7; and on Day 14. Tumors were measured approximately twice per week using calipers. Tumor volume was calculated using the formula X*Y*(X/2), where Y is the longest dimension and X is the perpendicular dimension, and mice with tumors larger than 2000 mm³ or with ulcerated tumors were euthanized by CO₂ asphyxiation. Survival was recorded until end of experiment at Day 70.

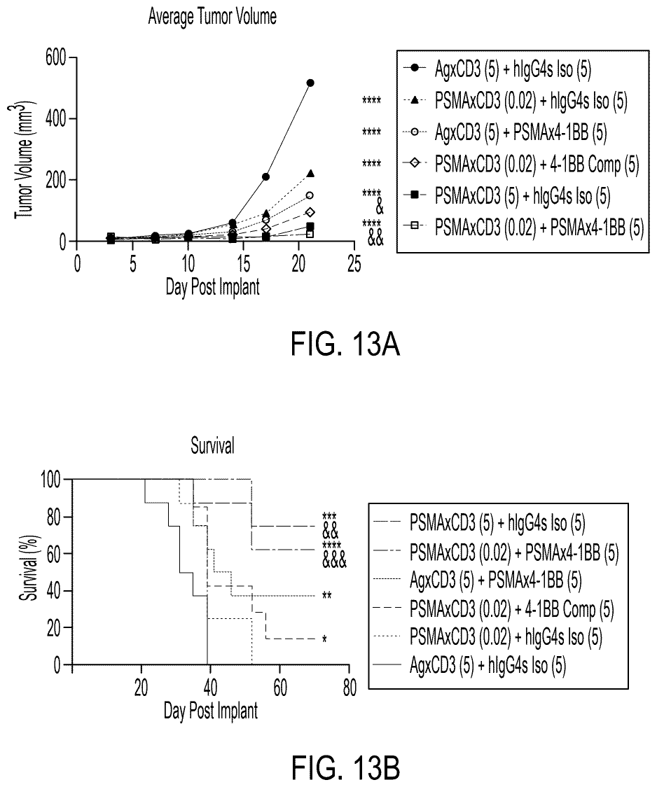

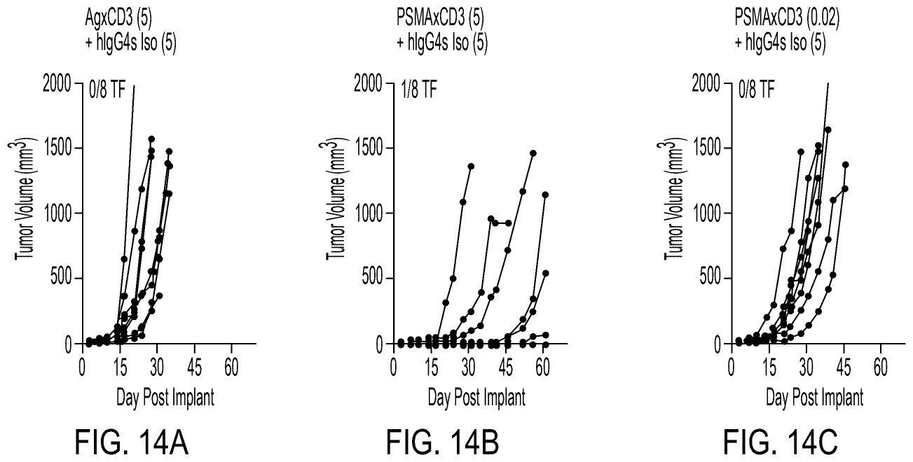

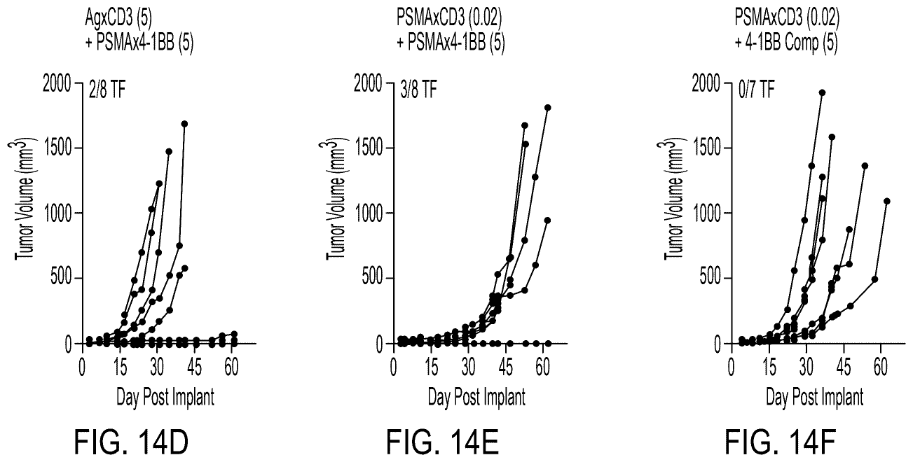

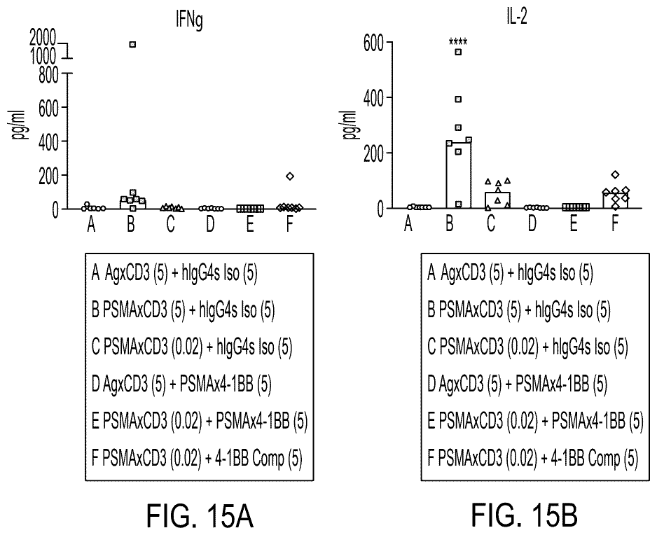

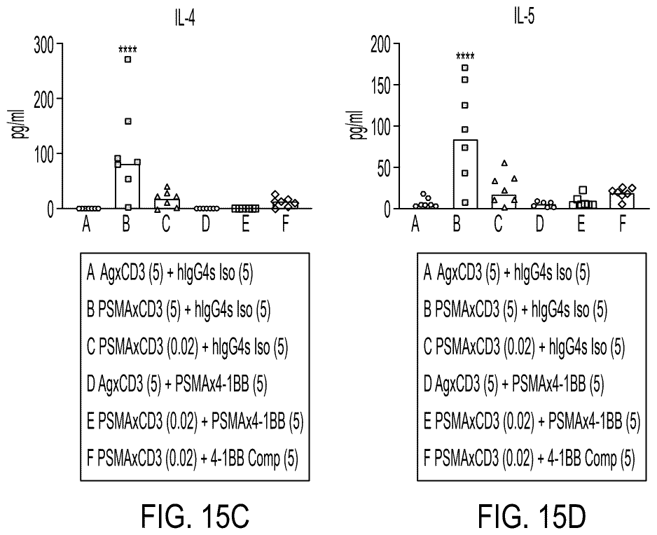

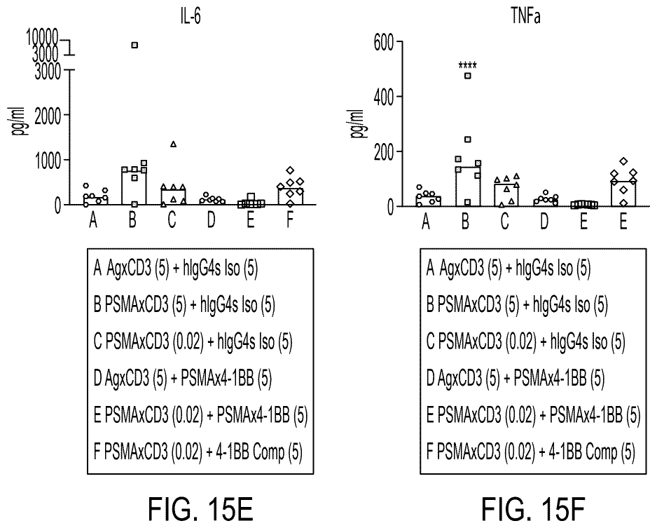

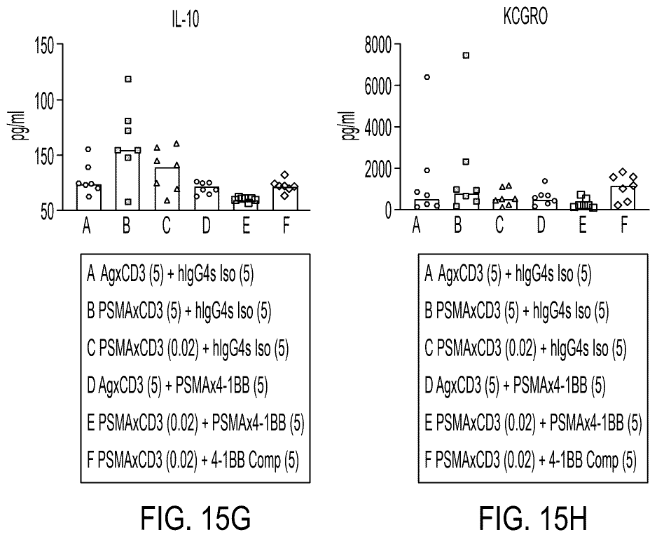

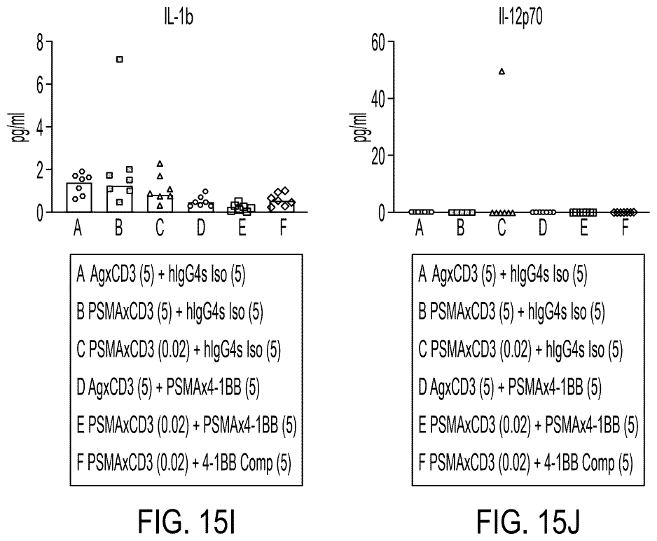

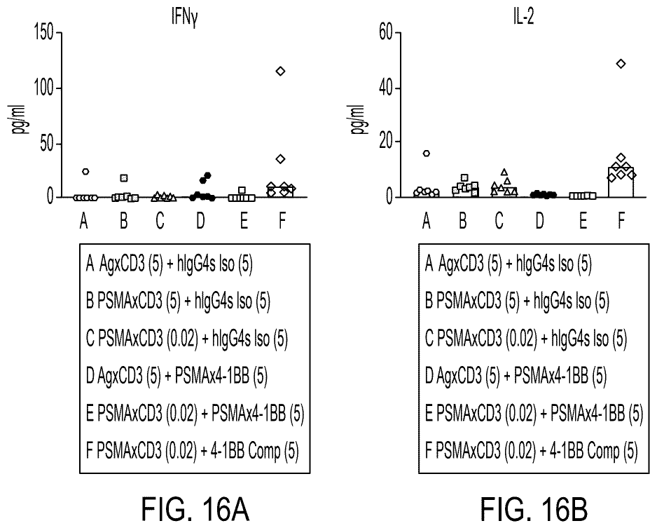

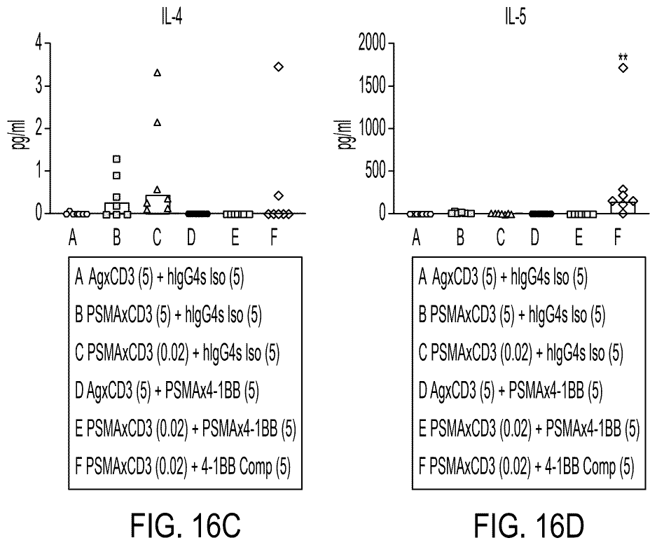

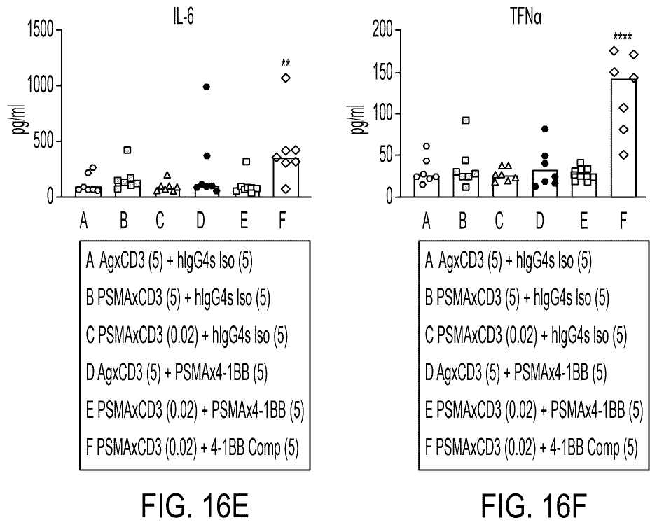

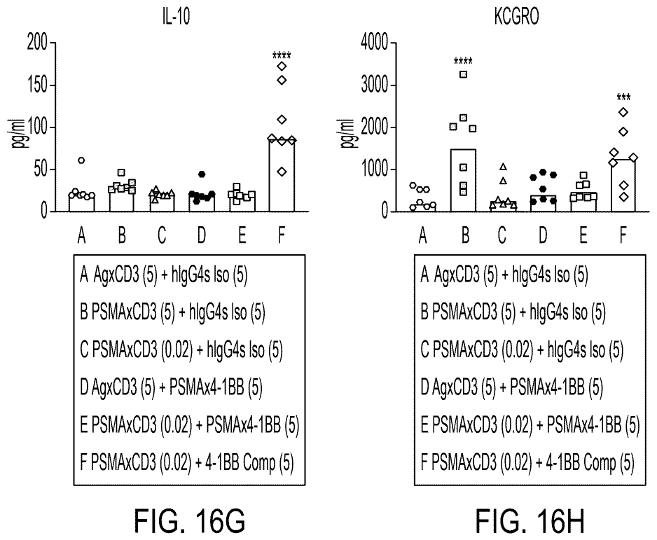

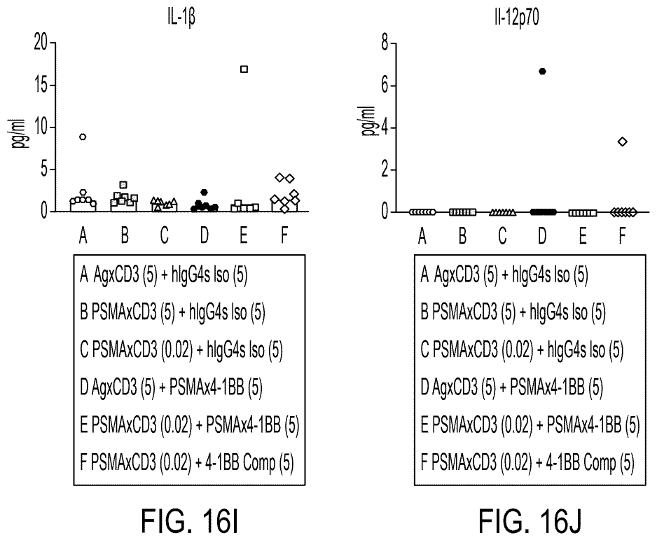

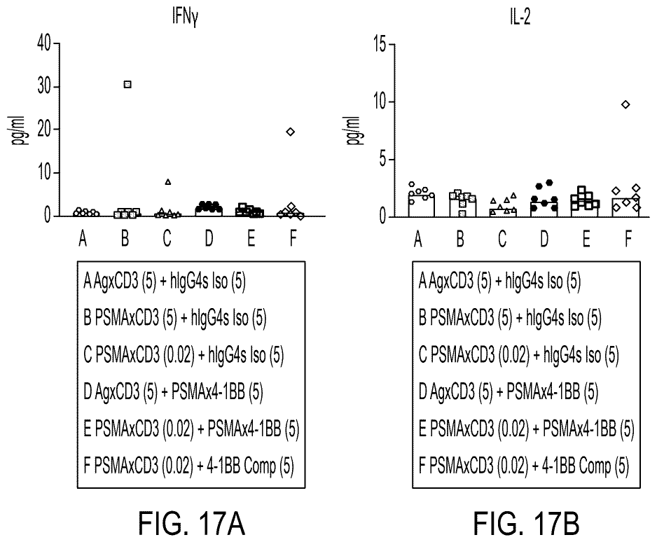

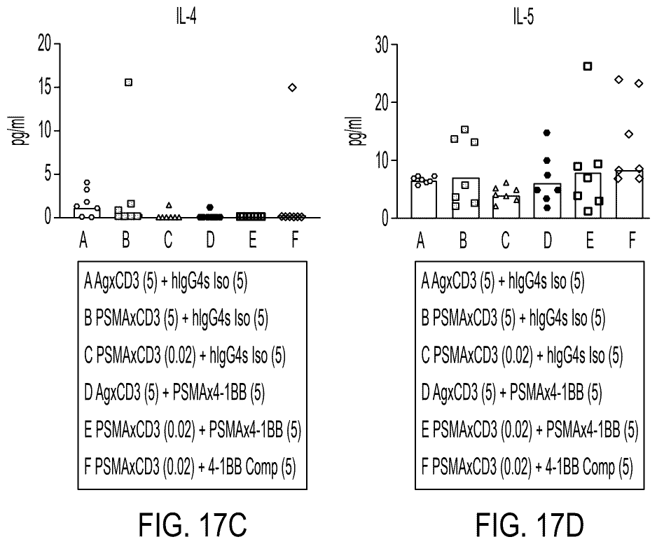

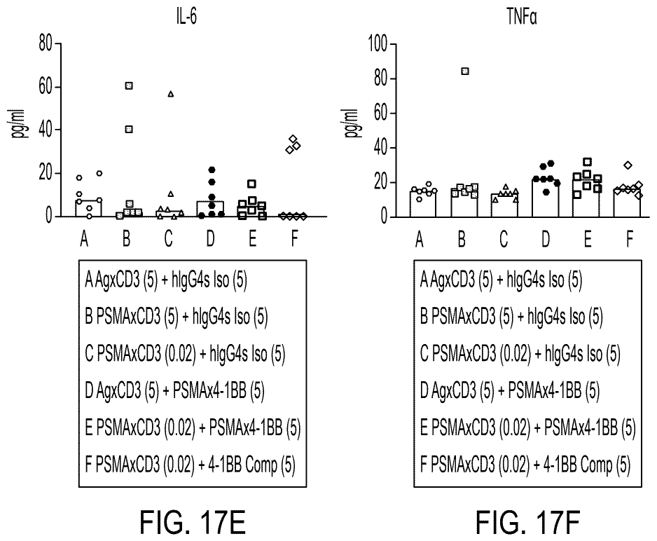

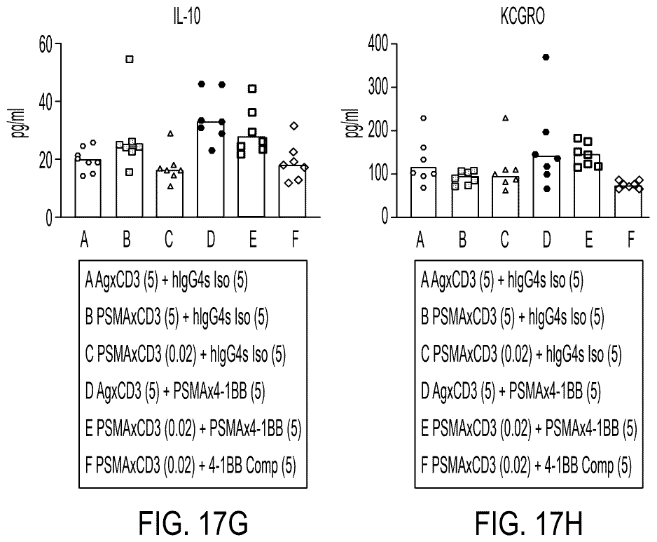

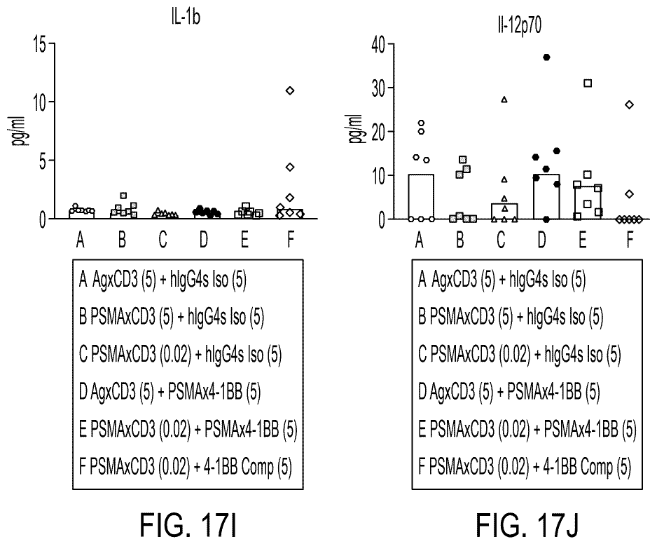

Results: Combination therapy of multispecific PSMAx4-1BB antibody and low dose PSMAxCD3 antibody shows robust anti-tumor efficacy and support of survival with negligible cytokine release. Administration of 5 mg/kg REGN15505 in combination with a low dose of PSMAxCD3 (0.02 mg/kg) resulted in greater control of tumor growth compared with either combination therapy of bivalent 4-1BB antibody and low dose PSMAxCD3 antibody or monotherapy of low dose PSMAxCD3 alone (FIG. 13A). For example, at Day 22 post implantation of Trampc2/hPSMA tumor cells, the average tumor volume of mice administered REGN15505 and low dose PSMAxCD3 combination therapy was only approximately 25 mm³, while the average tumor volume of mice administered bivalent 4-1BB antibody and low dose PSMAxCD3 combination therapy was approximately 100 mm³, and mice administered low dose PSMAxCD3 monotherapy showed an average tumor volume of approximately 225 mm³ (FIG. 13A). Regarding survival, for example, by Day 52 none of the 8 mice administrated low dose PSMAxCD3 monotherapy had survived (FIG. 14B). In contrast, 1 out of 7 mice administered the combination therapy of bivalent 4-1BB antibody and low dose PSMAxCD3 survived until end of experiment at Day 70. Surprisingly, 5 out of 8 mice administered the combination therapy of REGN15505 and low dose PSMAxCD3 survived until end of experiment at Day 70 (FIG. 13B). The rate of tumor rejection was also impressively higher for combination therapy of multispecific PSMAx4-1BB and low dose PSMAxCD3 (3/8 mice tumor free) (FIG. 14E) compared to combination therapy of bivalent 4-1BB antibody and low dose PSMAxCD3 (0/7 mice tumor free) (FIG. 14F) or low dose PSMAxCD3 monotherapy (0/8 mice tumor free) (FIG. 14C). The cytokine-release profile of the combination therapy of REGN15505 and low dose PSMAxCD3 was superior to combination therapy of bivalent 4-1BB antibody and low dose PSMAxCD3 for many cytokines at most time points (FIGS. 15A-17J).

As measured 4 hours postdose on Days 0 and 7, low-dose PSMAxCD3 in combination with REGN15505 did not increase the concentration in serum of any examined cytokine. In contrast, high-dose PSMAxCD3 alone led to increases in IFNG, TNFA, IL2, IL4, IL6 and IL10 on Day 0, and low-dose REGN15505+bivalent 4-1BB antibody led to increases in TNFA, IL5, and IL10 on Day 7.

Thus, REGN15505 in combination with low-dose PSMAxCD3 demonstrated similar anti-tumor efficacy to high-dose PSMAxCD3 but did not, unlike either high-dose PSMAxCD3 or low-dose REGN15505+bivalent 4-1BB antibody, increase concentrations in serum of the examined cytokines.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims. All patents, applications and non-patent publications mentioned in this specification are incorporated herein by reference in their entireties.

1. A multispecific antigen-binding molecule comprising:
(a) a first antigen-binding domain (D1) comprising three complementarity determining regions (CDRs) of a heavy chain variable region (D1-HCVR) and three CDRs of a light chain variable region (D1-LCVR), wherein the first antigen-binding domain binds specifically to PSMA; and
(b) a second antigen-binding domain (D2) comprising three CDRs of a HCVR (D2-HCVR) and three CDRs of a LCVR (D2-LCVR), wherein the second antigen-binding domain binds specifically to 4-1BB; and
(c) a third antigen-binding domain (D3) comprising three CDRs of a HCVR (D3-HCVR) and three CDRs of a LCVR (D3-LCVR), wherein the third antigen-binding domain binds specifically to 4-1BB.

2. The multispecific antigen-binding molecule of claim 1, wherein D2 and D3 bind to the same epitope on 4-1BB.

3. The multispecific antigen-binding molecule of claim 1, wherein D2 and D3 bind to different epitopes on 4-1BB.

4. The multispecific antigen-binding molecule of any one of claims 1-3, wherein D1 comprises three CDRs of a HCVR comprising an amino acid sequence selected from the group consisting of SEQ ID NOs: 2 and 32.

5. The multispecific antigen-binding molecule of any one of claims 1-4, wherein D1 comprises three CDRs of a LCVR comprising an amino acid sequence selected from the group consisting of SEQ ID NOs: 18 and 48.

6. The multispecific antigen-binding molecule of any one of claims 1-5, wherein D1 comprises three heavy chain complementarity determining regions (HCDR2, HCDR2, and HCDR3) comprising an amino acid sequence selected from the group consisting of SEQ ID NO: 4, SEQ ID NO: 6, and SEQ ID NO: 8; and SEQ ID NO: 34, SEQ ID NO: 36, and SEQ ID NO: 38, respectively.

7. The multispecific antigen-binding molecule of any one of claims 1-6, wherein D1 comprises three light chain complementarity determining regions (LCDR2, LCDR2, and LCDR3) comprising an amino acid sequence selected from the group consisting of SEQ ID NO: 20, GAS, and SEQ ID NO: 24; and SEQ ID NO: 50, AAS, and SEQ ID NO: 54, respectively.

8. The multispecific antigen-binding molecule of any one of claims 1-7, wherein D1 comprises a HCVR comprising the amino acid sequence selected from the group consisting of SEQ ID NOs: 2 and 32.

9. The multispecific antigen-binding molecule of any one of claims 1-8, wherein D1 comprises a LCVR comprising the amino acid sequence selected from the group consisting of SEQ ID NOs: 18 and 48.

10. The multispecific antigen-binding molecule of any one of claims 1-9, wherein D1 comprises a HCVR comprising the amino acid sequence selected from the group consisting of SEQ ID NOs: 2 and 32; and a LCVR comprising the amino acid sequence selected from the group consisting of SEQ ID NOs: 18 and 48.

11. The multispecific antigen-binding molecule of any one of claims 1-10, wherein D2 comprises three CDRs of a HCVR (D2-HCVR) comprising an amino acid sequence selected from the group consisting of SEQ ID NOs: 10 and 40.

12. The multispecific antigen-binding molecule of any one of claims 1-11, wherein D2 comprises three CDRs of a LCVR (D2-LCVR) comprising an amino acid sequence selected from the group consisting of SEQ ID NOs: 18 and 48.

13. The multispecific antigen-binding molecule of any one of claims 1-12, wherein D2 comprises three heavy chain complementarity determining regions (D2-HCDR1, D2-HCDR2, and D2-HCDR3) comprising an amino acid sequence selected from the group consisting of SEQ ID NO: 12, SEQ ID NO: 14, and SEQ ID NO: 16; and SEQ ID NO: 42, SEQ ID NO: 44, and SEQ ID NO: 46, respectively.

14. The multispecific antigen-binding molecule of any one of claims 1-13, wherein D2 comprises three light chain complementarity determining regions (D2-LCDR1, D2-LCDR2, and D2-LCDR3) comprising an amino acid sequence selected from the group consisting of SEQ ID NO: 20, GAS, and SEQ ID NO: 24; and SEQ ID NO: 50, AAS, and SEQ ID NO: 54, respectively.

15. The multispecific antigen-binding molecule of any one of claims 1-14, wherein D2 comprises a HCVR (D2-HCVR) comprising the amino acid sequence selected from the group consisting of SEQ ID NOs: 10 and 40.

16. The multispecific antigen-binding molecule of any one of claims 1-15, wherein D2 comprises a LCVR (D2-LCVR) comprising the amino acid sequence selected from the group consisting of SEQ ID NOs: 18 and 48.

17. The multispecific antigen-binding molecule of any one of claims 1-16, wherein D2 comprises a D2-HCVR comprising the amino acid sequence selected from the group consisting of SEQ ID NOs: 10 and 40; and a D2-LCVR comprising the amino acid sequence selected from the group consisting of SEQ ID NOs: 18 and 48.

18. The multispecific antigen-binding molecule of any one of claims 1-17, wherein D3 comprises three CDRs of a HCVR (D3-HCVR) comprising the amino acid sequence selected from the group consisting of SEQ ID NOs: 10 and 40.

19. The multispecific antigen-binding molecule of any one of claims 1-18, wherein D3 comprises three CDRs of a LCVR (D3-LCVR) comprising the amino acid sequence selected from the group consisting of SEQ ID NOs: 18 and 48.

20. The multispecific antigen-binding molecule any one of claims 1-19, wherein D3 comprises three heavy chain complementarity determining regions (D3-HCDR1, D3-HCDR2, and D3-HCDR3) comprising an amino acid sequence selected from the group consisting of SEQ ID NO: 12, SEQ ID NO: 14, and SEQ ID NO: 16; and SEQ ID NO: 42, SEQ ID NO: 44, and SEQ ID NO: 46, respectively.

21. The multispecific antigen-binding molecule of any one of claims 1-20, wherein D3 comprises three light chain complementarity determining regions (D3-LCDR1, D3-LCDR2, and D3-LCDR3) comprising an amino acid sequence selected from the group consisting of SEQ ID NO: 20, GAS, and SEQ ID NO: 24; and SEQ ID NO: 50, AAS, and SEQ ID NO: 54, respectively.

22. The multispecific antigen-binding molecule of any one of claims 1-21, wherein D3 comprises a HCVR (D3-HCVR) comprising the amino acid sequence selected from the group consisting of SEQ ID NOs: 10 and 40.

23. The multispecific antigen-binding molecule of any one of claim 1-22, wherein D3 comprises a LCVR (D3-LCVR) comprising the amino acid sequence selected from the group consisting of SEQ ID NOs: 18 and 48.

24. The multispecific antigen-binding molecule of any one of claims 1-23, wherein D3 comprises a D3-HCVR comprising the amino acid sequence selected from the group consisting of SEQ ID NOs: 10 and 40; and a D3-LCVR comprising the amino acid sequence selected from the group consisting of SEQ ID NOs: 18 and 48.

25. The multispecific antigen-binding molecule of any one of claims 1-24, wherein:
(a) the first antigen-binding domain (D1) comprises three CDRs of D1-HCVR comprising the amino acid sequence of SEQ ID NO: 2, and three CDRs of D1-LCVR comprising the amino acid sequence of SEQ ID NO: 18; and
(b) a second antigen-binding domain (D2) comprising three CDRs of D2-HCVR comprising an amino acid sequence selected from the group consisting of SEQ ID NOs: 10 and 40; and D2-LCVR comprising an amino acid sequence selected from the group consisting of SEQ ID NOs: 18 and 48; and
(c) a third antigen-binding domain (D3) comprising three CDRs of D3-HCVR comprising an amino acid sequence selected from the group consisting of SEQ ID NOs: 10 and 40 and D3-LCVR comprising an amino acid sequence selected from the group consisting of SEQ ID NOs: 18 and 48.

26. The multispecific antigen-binding molecule of any one of claims 1-24, wherein:
(a) the first antigen-binding domain (D1) comprises three CDRs of D1-HCVR comprising the amino acid sequence of SEQ ID NO: 32, and three CDRs of D1-LCVR comprising the amino acid sequence of SEQ ID NO: 48; and
(b) a second antigen-binding domain (D2) comprising three CDRs of D2-HCVR comprising an amino acid sequence selected from the group consisting of SEQ ID NOs: 10 and 40; and D2-LCVR comprising an amino acid sequence selected from the group consisting of SEQ ID NOs: 18 and 48; and
(c) a third antigen-binding domain (D3) comprising three CDRs of D3-HCVR comprising an amino acid sequence selected from the group consisting of SEQ ID NOs: 10 and 40 and D3-LCVR comprising an amino acid sequence selected from the group consisting of SEQ ID NOs: 18 and 48.

27. The multispecific antigen-binding molecule of any one of claims 1-24, wherein:
(a) the first antigen-binding domain (D1) comprises three CDRs of D1-HCVR comprising the amino acid sequence of SEQ ID NO: 2, and three CDRs of D1-LCVR comprising the amino acid sequence of SEQ ID NO: 18; and
(b) the second antigen-binding domain (D2) comprises three CDRs of D2-HCVR comprising the amino acid sequence of SEQ ID NO: 10; and three CDRs of D2-LCVR comprising the amino acid sequence of SEQ ID NO: 18; and
(c) a third antigen-binding domain (D3) comprising three CDRs of D3-HCVR comprising the amino acid sequence of SEQ ID NO: 10; and three CDRs of D3-LCVR comprising the amino acid sequence of SEQ ID No: 18.

28. The multispecific antigen-binding molecule of claim 27, wherein:
(a) the first antigen-binding domain (D1) comprises D1-HCDR1, D1-HCDR2, D1-HCDR3, D1-LCDR1, D1-LCDR2, and D1-LCDR3 comprising the respective amino acid sequences of SEQ ID NO: 4, SEQ ID NO: 6, SEQ ID NO: 8, SEQ ID NO: 20, GAS, and SEQ ID NO: 24; and
(b) the second antigen-binding domain (D2) comprises D2-HCDR1, D2-HCDR2, D2-HCDR3, D2-LCDR1, D2-LCDR2, and D2-LCDR3 comprising the respective amino acid sequences of SEQ ID NO: 12, SEQ ID NO: 14, SEQ ID NO: 16, SEQ ID NO: 20, GAS, and SEQ ID NO: 24; and
(c) a third antigen-binding domain (D3) comprising D3-HCDR1, D3-HCDR2, D3-HCDR3, D3-LCDR1, D3-LCDR2, and D3-LCDR3 comprising the respective amino acid sequences of SEQ ID NO: 12, SEQ ID NO: 14, SEQ ID NO: 16, SEQ ID NO: 20, GAS, and SEQ ID NO: 24.

29. The multispecific antigen-binding molecule of any one of claims 1-24, wherein:
(a) the first antigen-binding domain (D1) comprises three CDRs of D1-HCVR comprising the amino acid sequence of SEQ ID NO: 32, and three CDRs of D1-LCVR comprising the amino acid sequence of SEQ ID NO: 48; and
(b) the second antigen-binding domain (D2) comprises three CDRs of D2-HCVR comprising the amino acid sequence of SEQ ID NO: 40; and three CDRs of D2-LCVR comprising the amino acid sequence of SEQ ID NO: 48; and
(c) a third antigen-binding domain (D3) comprising three CDRs of D3-HCVR comprising the amino acid sequence of SEQ ID NO: 40; and three CDRs of D3-LCVR comprising the amino acid sequence of SEQ ID No: 48.

30. The multispecific antigen-binding molecule of claim 29, wherein:
(a) the first antigen-binding domain (D1) comprises D1-HCDR1, D1-HCDR2, D1-HCDR3, D1-LCDR1, D1-LCDR2, and D1-LCDR3 comprising the respective amino acid sequences of SEQ ID NO: 34, SEQ ID NO: 36, SEQ ID NO: 38, SEQ ID NO: 50, AAS, and SEQ ID NO: 54; and
(b) the second antigen-binding domain (D2) comprises D2-HCDR1, D2-HCDR2, D2-HCDR3, D2-LCDR1, D2-LCDR2, and D2-LCDR3 comprising the respective amino acid sequences of SEQ ID NO: 42, SEQ ID NO: 44, SEQ ID NO: 46, SEQ ID NO: 50, AAS, and SEQ ID NO: 54; and
(c) a third antigen-binding domain (D3) comprising D3-HCDR1, D3-HCDR2, D3-HCDR3, D3-LCDR1, D3-LCDR2, and D3-LCDR3 comprising the respective amino acid sequences of SEQ ID NO: 42, SEQ ID NO: 44, SEQ ID NO: 46, SEQ ID NO: 50, AAS, and SEQ ID NO: 54

31. The multispecific antigen-binding molecule of any one of claims 1-30, wherein the molecule is a multispecific antibody.

32. The multispecific antigen-binding molecule of claim 31 comprising:
(a) a first antigen-binding arm comprising the first antigen-binding domain (D1) comprising three CDRs (D1-HCDR1, D1-HCDR2, and D1-HCDR3) of the HCVR (D1-HCVR) and three CDRs (D1-LCDR1, D1-LCDR2, and D1-LCDR3) of the LCVR (D1-LCVR), wherein the first antigen-binding arm binds specifically to PSMA; and
(b) a second antigen-binding arm comprising (i) the second antigen-binding domain (D2) comprising three CDRs (D2-HCDR1, D2-HCDR2, and D2-HCDR3) of the HCVR (D2-HCVR) and three CDRs (D2-LCDR1, D2-LCDR2, and D2-LCDR3) of the LCVR (D2-LCVR); and (ii) the third antigen-binding domain (D3) comprising three CDRs (D3-HCDR1, D3-HCDR2, and D3-HCDR3) of the HCVR (D3-HCVR) and three CDRs (D3-LCDR1, D3-LCDR2, and D3-LCDR3) of the HCVR (D3-LCVR), wherein the second antigen-binding arm binds specifically to 4-1BB.

33. The multispecific antigen-binding molecule of claim 32, wherein the multispecific molecule comprises a heavy chain, wherein the heavy chain constant region is of IgG1 or IgG4 isotype.

34. The multispecific antigen-binding molecule of claim 32 or 33, wherein the multispecific molecule comprises a first heavy chain comprising D1-HCVR, and a second heavy chain comprising D2-HCVR and D3-HCVR; wherein the second heavy chain comprises the mutations H435R and Y436F (EU numbering).

35. The multispecific antigen-binding molecule of claim 32 or 33 comprising a first heavy chain comprising D1-HCVR of the first antigen-binding arm paired with a first light chain comprising D1-LCVR of the first antigen-binding arm, wherein the first heavy chain comprises the amino acid sequence of SEQ ID NO: 26, and the first light chain comprises the amino acid sequence of SEQ ID NO: 30.

36. The multispecific antigen-binding molecule of claim 35 comprising a second heavy chain comprising D2-HCVR and D3-HCVR of the second antigen-binding arm paired with a second light chain comprising D2-LCVR and a third light chain comprising D3-LCVR, wherein the second heavy chain comprises the amino acid sequence of SEQ ID NO: 28; the second light chain comprises the amino acid sequence of SEQ ID NO: 30, and the third light chain comprises the amino acid sequence of SEQ ID NO: 30.

37. The multispecific antigen-binding molecule of claim 32 or 33 comprising a first heavy chain comprising D1-HCVR of the first antigen-binding arm paired with a first light chain comprising D1-LCVR of the first antigen-binding arm, wherein the first heavy chain comprises the amino acid sequence of SEQ ID NO: 56, and the first light chain comprises the amino acid sequence of SEQ ID NO: 60.

38. The multispecific antigen-binding molecule of claim 37 comprising a second heavy chain comprising D2-HCVR and D3-HCVR of the second antigen-binding arm paired with a second light chain comprising D2-LCVR and a third light chain comprising D3-LCVR, wherein the second heavy chain comprises the amino acid sequence of SEQ ID NO: 58; the second light chain comprises the amino acid sequence of SEQ ID NO: 60, and the third light chain comprises the amino acid sequence of SEQ ID NO: 60.

39. The multispecific antigen-binding molecule of claim 32 or 33, wherein:
(a) the first antigen-binding arm comprises a heavy chain comprising the sequence of SEQ ID NO: 26 and a first light chain comprising the sequence of SEQ ID NO 30; and
(b) the second antigen-binding arm comprises a heavy chain comprising the sequence of SEQ ID NO: 28, a second light chain comprising the sequence of SEQ ID NO: 30, and a third light chain comprising the sequence of SEQ ID NO: 30.

40. The multispecific antigen-binding molecule of claim 32 or 33, wherein:
(a) the first antigen-binding arm comprises a heavy chain comprising the sequence of SEQ ID NO: 56 and a first light chain comprising the sequence of SEQ ID NO 60; and
(b) the second antigen-binding arm comprises a heavy chain comprising the sequence of SEQ ID NO: 58, a second light chain comprising the sequence of SEQ ID NO: 60, and a third light chain comprising the sequence of SEQ ID NO: 60.

41. A multispecific antigen-binding molecule comprising a first antigen-binding arm that binds specifically to PSMA and a second antigen-binding arm that binds specifically to 4-1BB, wherein:
(a) the first antigen-binding arm comprises a first antigen-binding domain (D1) comprising three CDRs of a HCVR (D1-HCVR) comprising the amino acid sequence of SEQ ID NO: 2, and three CDRs of a LCVR (D1-LCVR) comprising the amino acid sequence of SEQ ID NO: 18; and
(b) the second antigen-binding arm comprises a second antigen-binding domain (D2) comprising (i) three CDRs of a HCVR (D2-HCVR) comprising the amino acid sequence of SEQ ID NO: 10, and three CDRs of a LCVR (D2-LCVR) comprising the amino acid sequence of SEQ ID NO: 18, and (ii) a third antigen-binding domain (D3) comprising (i) three CDRs of a HCVR (D3-HCVR) comprising the amino acid sequence of SEQ ID NO: 10, and three CDRs of a LCVR (D3-LCVR) comprising the amino acid sequence of SEQ ID NO: 18.

42. A multispecific antigen-binding molecule comprising a first antigen-binding arm that binds specifically to PSMA and a second antigen-binding arm that binds specifically to 4-1BB, wherein:
(a) the first antigen-binding arm comprises a first antigen-binding domain (D1) comprising three CDRs of a HCVR (D1-HCVR) comprising the amino acid sequence of SEQ ID NO: 32, and three CDRs of a LCVR (D1-LCVR) comprising the amino acid sequence of SEQ ID NO: 48; and
(b) the second antigen-binding arm comprises a second antigen-binding domain (D2) comprising (i) three CDRs of a HCVR (D2-HCVR) comprising the amino acid sequence of SEQ ID NO: 40, and three CDRs of a LCVR (D2-LCVR) and comprising the amino acid sequence of SEQ ID NO: 48, and (ii) a third antigen-binding domain (D3) comprising (i) three CDRs of a HCVR (D3-HCVR) comprising the amino acid sequence of SEQ ID NO: 40, and three CDRs of a LCVR (D3-LCVR) comprising the amino acid sequence of SEQ ID NO: 48.

43. The multispecific antigen-binding molecule of claim 41 or 42, wherein the molecule is a multispecific antibody.

44. The multispecific antigen-binding molecule of claim 41, wherein the molecule is a multispecific antibody comprising a first heavy chain comprising the HCVR of the first antigen-binding arm, wherein the first heavy chain is paired with a light chain comprising the LCVR of the first antigen-binding arm, wherein the first heavy chain comprises the amino acid sequence of SEQ ID NO: 26 and the light chain comprises the amino acid sequence of SEQ ID NO: 30.

45. The multispecific antigen-binding molecule of claim 41 or 44, wherein the molecule is a multispecific antibody comprising a second heavy chain comprising D2-HCVR and D3-HCVR of the second antigen-binding arm paired with a second light chain comprising D2-LCVR and a third light chain comprising D3-LCVR, wherein the second heavy chain comprises the amino acid sequence of SEQ ID NO: 28; the second light chain comprises the amino acid sequence of SEQ ID NO: 30, and the third light chain comprises the amino acid sequence of SEQ ID NO: 30.

46. The multispecific antigen-binding molecule of claim 42, wherein the molecule is a multispecific antibody comprising a first heavy chain comprising the HCVR of the first antigen-binding arm, wherein the first heavy chain is paired with a light chain comprising the LCVR of the first antigen-binding arm, wherein the first heavy chain comprises the amino acid sequence of SEQ ID NO: 56 and the light chain comprises the amino acid sequence of SEQ ID NO: 60.

47. The multispecific antigen-binding molecule of claim 42 or 46, wherein the molecule is a multispecific antibody comprising a second heavy chain comprising D2-HCVR and D3-HCVR of the second antigen-binding arm paired with a second light chain comprising D2-LCVR and a third light chain comprising D3-LCVR, wherein the second heavy chain comprises the amino acid sequence of SEQ ID NO: 58; the second light chain comprises the amino acid sequence of SEQ ID NO: 60, and the third light chain comprises the amino acid sequence of SEQ ID NO: 60.

48. A pharmaceutical composition comprising the multispecific antigen-binding molecule of any one of claims 1-47, and a pharmaceutically acceptable carrier.

49. An isolated nucleic acid molecule comprising a polynucleotide sequence encoding an amino acid sequence comprising D1-HCVR of the multispecific antigen-binding molecule of any one of claims 1-47.

50. An isolated nucleic acid molecule comprising a nucleic acid sequence encoding an amino acid sequence comprising D2-HCVR and D3-HCVR of the multispecific antigen-binding molecule of any one of claims 1-47.

51. An isolated nucleic acid molecule comprising a nucleic acid sequence encoding an amino acid sequence comprising one or more LCVRs of the group consisting of D1-LCVR, D2-LCVR, and D3-LCVR of the multispecific antigen-binding molecule of any one of claims 1-47.

52. An expression vector containing an isolated nucleic acid molecule of any one of claims 49-51; or a set of expression vectors containing a set of nucleic acid molecules comprising nucleic acid sequences encoding amino acid sequences comprising D1-HCVR, D2-HCVR, D3-HCVR, D1-LCVR, D2-LCVR and/or D3-LCVR of the multispecific antigen-binding molecule of any one of claims 1-47.

53. A host cell containing the expression vector or set of expression vectors of claim 52.

54. The host cell of claim 53, wherein the host cell is E. coli or CHO cell.

55. A method of producing a multispecific antigen-binding molecule, the method comprising growing the host cell of claim 53 under conditions permitting production of the multispecific antigen-binding molecule, wherein the host cell comprises a first nucleic acid molecule comprising a nucleic acid sequence encoding an amino acid sequence comprising a first antigen-binding arm comprising a heavy chain variable region (HCVR) of the multispecific antigen-binding molecule, a second nucleic acid molecule comprising a nucleic acid sequence encoding a second amino acid sequence comprising a second antigen-binding arm comprising heavy chain variable regions (HCVRs) of the multispecific antigen-binding molecule, and a third nucleic acid molecule comprising a nucleic acid sequence encoding an amino acid sequence comprising a common light chain variable region (LCVR).

56. The method of claim 55, wherein the host cell comprises a first nucleic acid molecule comprising a nucleic acid sequence encoding an amino acid sequence comprising a heavy chain of the first antigen-binding arm of the multispecific antigen-binding molecule, a second nucleic acid molecule comprising a nucleic acid sequence encoding an amino acid sequence comprising a heavy chain of the second antigen-binding arm of the multispecific antigen-binding molecule, and a third nucleic acid molecule comprising a nucleic acid sequence encoding an amino acid sequence comprising a common light chain.

57. A method of inhibiting growth of a tumor in a subject, comprising administering a multispecific antigen-binding molecule of any one of claims 1-47 to the subject.

58. The method of claim 57, wherein the tumor is a prostate cancer tumor.

59. A method of inhibiting growth of a tumor in a subject, the method comprising administering a multispecific antigen-binding molecule of any one of claims 1-47 to the subject, wherein the tumor is selected from the group consisting of prostate cancer, hepatocellular carcinoma, non-small cell lung cancer, melanoma, pancreatic ductal adenocarcinoma, glioma, or breast cancer, or a cancer characterized in part by having PSMA+ cells.

60. The method of any one of claims 57-59, further comprising administering an additional therapeutic agent or therapeutic regimen.

61. The method of claim 60, wherein the additional therapeutic is an anti-PSMA x anti-CD3 multispecific antigen-binding molecule.

62. The method of claim 60, wherein the additional therapeutic agent or therapeutic regimen comprises a chemotherapeutic drug, a DNA alkylator, an immunomodulator, a proteasome inhibitor, a histone deacetylase inhibitor, radiotherapy, a stem cell transplant, surgery, a different multispecific antibody that interacts with a different tumor cell surface antigen and a T cell or immune cell antigen, an antibody drug conjugate, a multispecific antibody conjugated to an anti-tumor agent, a checkpoint inhibitor, a PD-1 inhibitor, a PD-L1 inhibitor, a CTLA-4 inhibitor, a CD28 agonist, a 4-1BB agonist, a radioligand, hormone therapy, a CD3 blocker, a STEAP1 inhibitor, an EGFR inhibitor, a GITR agonist, a cancer vaccine, an oncolytic virus, an immunocytokine, IL4 inhibitor, IL6 inhibitor, IL2 or modified IL2, IL12, Il10, IL23, a T cell comprising a chimeric antigen receptor (CAR-T cell), or combinations thereof.

63. The method of any one of claims 57-62, wherein the subject has a PSMA+ tumor.

64. The method of claim 62, wherein the additional therapeutic agent is a PD-1 inhibitor, wherein the PD-1 inhibitor is an anti-PD-1 antibody or antigen-binding fragment thereof, or an anti-PD-L1 antibody or antigen-binding fragment thereof.

65. The method of claim 64, wherein the anti-PD-1 inhibitor is selected from cemiplimab, nivolumab, pembrolizumab, durvalumab, atezolizumab, and avelumab.

66. The method of claim 64, wherein the PD-1 inhibitor is cemiplimab.

67. Use of a multispecific antigen-binding molecule of any one of claims 1-47 in the treatment of a disease or disorder associated with expression of PSMA.

68. The use of claim 67, wherein the disease or disorder is cancer.

69. The use of claim 67 or 68, wherein the cancer is prostate cancer, hepatocellular carcinoma, non-small cell lung cancer, melanoma, pancreatic ductal adenocarcinoma, glioma, breast cancer, or another cancer characterized in part by having PSMA+ cells.

70. The use of any one of claims 67-69, wherein the multispecific antigen-binding molecule or pharmaceutical composition is for use in combination with an additional therapeutic agent or therapy.

71. The use of any one of claims 67-70, wherein the multispecific antigen-binding molecule or pharmaceutical composition is injected intravenously, intramuscularly, or subcutaneously.
